(19) United States
(12) Patent Application Publication
(10) Pub. No.: US 2021/0186905 A1
(43) Pub. Date: Jun. 24, 2021

(54) ENHANCING T-CELL FUNCTION AND TREATING A T-CELL DYSFUNCTIONAL DISORDER WITH A COMBINATION OF AN LSD INHIBITOR AND A PD-1 BINDING ANTAGONIST

(71) Applicant: EpiAxis Therapeutic Pty Ltd, Herston (AU)

(72) Inventor: Sudha RAO, Red Hill (AU)

(21) Appl. No.: 16/768,578

(22) PCT Filed: Nov. 28, 2018
(86) PCT No.: PCT/AU2018/051268

§ 371 Date: May 29, 2020

(30) Foreign Application Priority Data
Nov. 29, 2017 (AU) 2017904811

(51) Int. Cl. A61K 31/166; A61K 47/54; A61K 39/395; A61K 31/337; A61K 47/64; A61P 37/04; A61P 35/04; A61P 31/22; G01N 33/50; G01N 33/68; A61K 31/15

(52) U.S. Cl. CPC A61K 31/166; A61K 47/543; A61K 39/3955; A61K 31/337; A61K 47/64; A61P 37/04; G01N 2800/7028; A61P 31/22; G01N 33/505; G01N 33/5091; G01N 33/6875; A61K 31/15; G01N 2800/44; A61P 35/04

(57) ABSTRACT

The present invention relates to a composition for enhancing T-cell function or for treating a T-cell dysfunctional disorder, the composition comprising, consisting or consisting essentially of a lysine specific demethylase (LSD) inhibitor (which may be a MAO inhibitor or phenelzine) and a Programmed cell death protein-1 (PD-1) binding antagonist (which may be an antibody, preferably nivolumab, pembrolizumab, lambrolizumab or pidilizumab).

### FIELD OF THE INVENTION

This application claims priority to Australian Provisional Application No. 2017904811 entitled “Immunopotentiating compositions and uses therefor” filed 29 Nov. 2017, the contents of which are incorporated herein by reference in their entirety.

This invention relates generally to immunopotentiating compositions. More particularly, the present invention relates to the use of lysine demethylase (LSD) inhibitors for enhancing the immune effector function of functionally repressed T-cells that have undergone epithelial to mesenchymal transition (EMT). In specific embodiments, LSD inhibitors are used to enhance susceptibility of exhausted T-cells to reinvigoration by PD-1 binding antagonists. The compositions of the present invention find utility in treating a range of disorders including T-cell dysfunctional disorders such as pathogenic infections and hyperproliferative disorders.

### BACKGROUND OF THE INVENTION

Programmed death receptor 1 (PD-1) is an immune checkpoint regulator that is expressed in various immune cells including T-cells, B-cells, natural killer (NK) cells, NK T (NKT) cells, monocytes, macrophages, and dendritic cells (DCs) following their activation. PD-1 binds to its two ligands: programmed cell death 1 ligand-1 (PD-L1; B7-H1; CD274) and PD-L2 (B7-DC; CD273), both of which are B7 family members. PD-L1 is constitutively expressed in a wide range of cells including hematopoietic and non-hematopoietic cells. In contrast, PD-L2 expression is restricted to professional antigen presenting cells (APCs; monocytes, macrophages, and DCs) and a certain subset of B cells. Inflammatory cytokines such as interferons (IFNs; α, β, and γ) are potent regulators of both PD-L1 and PD-L2 expression.

PD-1 is induced by T-cell receptor (TCR) signaling, and when PD-1 binds to PD-L1 or PD-L2, it inhibits TCR/CD28 signaling and T-cell activation. These immunoregulatory roles of PD-1 are responsible for limiting excessive T-cell activation to prevent immune-mediated tissue damage. However, prolonged TCR stimulation and PD-1 expression lead to T-cell exhaustion, which is a state of T-cell dysfunction defined by poor T-cell effector function, sustained expression of inhibitory receptors and a transcriptional state distinct from that of functional effector or memory T-cells, and which is commonly associated with inefficient control of tumors and persistent viral infections (Wherry, E J, 2011. Nature Immunology 12: 492-499). As such, the PD-1 pathway is an important determinant of the outcome of the T-cell response, regulating the balance between effective host defense and immunopathology, implicating the potential for manipulating the PD-1 pathway against various human diseases.

Blockade of the PD-1 pathway has been used to reinvigorate exhausted T-cells and restore anti-tumor or anti-pathogen immune responses. Indeed, antibodies that block the PD-1 pathway have shown promising clinical results in a significant number of advanced-stage cancer patients. However, clinical trial data to date show a high variety of response rates among different types of cancers to PD-1 immune checkpoint inhibition therapy, with a range of 18% to 87%. These trials have also found that patients can present with primary, adaptive, or even acquired resistance to PD-1 immune-checkpoint inhibition therapy. Furthermore, emerging data demonstrate that certain patients experience hyperprogressive disease status after receiving anti-PD-1 antibodies. [symbol] ecently, Huang et al. (2017, Nature 545: 60-65) used im [symbol] peripheral blood from patients with stage IV melanoma before and after treatment with the anti-PD-1 antibody, pembrolizumab, to identify pharmacodynamic changes in circulating exhausted-phenotype CD8 T-cells (Tₑₓ cells). Most of the patients demonstrated an immunological response to pembrolizumab but this was short lived. Clinical failure in many patients was not solely due to an inability to induce immune reinvigoration, but rather resulted from an imbalance between T-cell reinvigoration and tumor burden. The magnitude of reinvigoration of circulating Tₑₓ cells determined in relation to pretreatment tumor burden correlated with clinical response, raising the possibility that even robust reinvigoration by anti-PD-1 therapy may be clinically ineffective if the tumor burden is high.

### SUMMARY OF THE INVENTION

The present invention arises from the unexpected finding that increased translocation of phosphorylated lysine demethylase 1 (also referred to herein as “LSD1p” or “nuclear LSD”) in the nucleus of T-cells (e.g., CD8⁺ T-cells) induces epithelial to mesenchymal transition (EMT) of the cells with repression of their immune effector function, including decreased expression of biomarkers of T-cell activation and effector capacity (e.g., interleukin-2 (IL-2), interferon-γ (IFN-γ) and tumor necrosis factor-α (TNF-α)), reduced expression of the transcription factor TBET, which has a role in stimulating production of IFN-γ in cells of the adaptive and innate immune systems, and increased expression of Eomesodermin (EOMES), which is a biomarker of T-cell exhaustion. Unexpectedly, LSD1p and EOMES were found to co-localize in the nucleus and that this co-localization contributes at least in part to repression of T-cell function. Notably, the inventors have determined that LSD1p and EOMES are in close proximity in the nucleus and form a complex that is predicted to be a repressor of T-cell function, including stimulating the T-cell to adopt and/or maintain an exhausted phenotype.

The present inventors have also found that exposure of these mesenchymal, functionally repressed T-cells to LSD inhibitors, including LSD1 inhibitors, results in epigenetic reprogramming of the T-cells with remarkable de-repression of their immune effector function, including elevated expression of biomarkers of T-cell activation and effector capacity (e.g., IFN-γ, TNF-α, Ki67 and TBET), decreased expression of biomarkers of T-cell exhaustion (e.g., EOMES), as well as increased activation and proliferation of T-cells, including effector and memory T-cells. Surprisingly, it has also been found that LSD inhibitor-mediated epigenetic reprogramming confers enhanced susceptibility of exhausted T-cells to reinvigoration by PD-1 binding antagonists. These findings have been reduced to practice in methods and compositions for enhancing the immune effector function of T-cells and for treating diseases or conditions associated with T-cell dysfunction, as described hereafter.

Accordingly, in one aspect, the present invention provides compositions for enhancing T-cell (e.g., CD8⁺ T-cell or CD4⁺ T-cell) function, or for treating a T-cell dysfunctional disorder. These compositions generally comprise, consist or consist essentially of a LSD inhibitor and a PD-1 binding antagonist. The LSD inhibitor is suitably selected from inhibitors of LSD enzymatic activity and inhibitors of LSD nuclear translocation. The LSD inhibitor is suitably a LSD1 inhibitor and in specific examples, the LSD1 inhibitor is a specific or selective LSD1 inhibitor. The PD-1 binding antagonist suitably inhibits the binding of PD-1 to PD-L1 and/or PD-L2. In preferred embodiments, the PD-1 binding antagonist is an anti-PD-1 antagonist antibody, illustrative examples of which include nivolumab, pembrolizumab, lambrolizumab and pidilizumab. In other embodiments, the PD-1 binding antagonist is an immunoadhesin (e.g., AMP-224). In specific [symbol] e compositions further comprise an ancillary agent for tre [symbol] n the treatment of, a T-cell dysfunctional disorder. In advantageous embodiments of this type, the ancillary agent is a chemotherapeutic agent, which suitably targets rapidly dividing cells and/or disrupt the cell cycle or cell division (e.g., a cytotoxic compound such as a taxane). The compositions are typically pharmaceutical compositions or formulations, which optionally comprise a pharmaceutically acceptable carrier.

Another aspect of the present invention provides methods of enhancing T-cell function. These methods generally comprise, consist or consist essentially of contacting a T-cell with a LSD inhibitor (e.g., a LSD1 inhibitor) and a PD-1 binding antagonist, to thereby enhance T-cell function. Suitably, the enhanced T-cell function includes any one or more of elevated biomarkers of T-cell activation and effector capacity (e.g., IFN-γ, TNF-α, Ki67 and TBET), increased proliferation of T-cells, including effector T-cells and/or memory T-cells, increased activation of T-cells including CD4⁺ and CD8⁺ T-cells, increased recognition of an antigen or an antigen peptide derived from an antigen in the context of MHC class II molecules by T-cell receptors, increased recognition of an antigen or an antigen peptide derived from an antigen in the context of MHC class I molecules by T-cell receptors, increased elimination of cells presented in the context of MHC class I molecules and increased cytolytic killing of antigen expressing target cells. In some embodiments, the T-cell has a mesenchymal phenotype. Suitably, the T-cell has aberrant expression of nuclear LSD. In representative examples of this type, the T-cell expresses nuclear LSD at a higher level than the level of expression of TBET in the same T-cell, and/or at a higher level than in an activated T-cell. In some of the same and other embodiments, the T-cell is one exhibiting T-cell exhaustion or anergy. In non-limiting examples of this type, the T-cell expresses a higher level of EOMES than TBET and/or has elevated expression of PD-1. The T-cell may be a CD4⁺ T-cell or a CD8⁺ T-cell. Preferably, the T-cell is a CD8⁺ T-cell.

The present inventors propose that since nuclear LSD-mediated EMT occurs both in tumor cells and in T-cells, which are unrelated cell types, nuclear LSD-mediated epigenetic reprogramming is also likely to occur more broadly, including in other immune effector cells that express PD-1 (e.g., T-cells, B-cells, NK cells, NKT cells, monocytes, macrophages and DCs), to thereby repress their immune effector function. Accordingly, in another aspect, the present invention provides methods of enhancing immune effector function of an immune effector cell that expresses PD-1. These methods generally comprise, consist or consist essentially of contacting the immune effector cell with a LSD inhibitor (e.g., a LSD1 inhibitor) and a PD-1 binding antagonist, to thereby enhance the immune effector function of the immune effector cell. Suitably, the enhanced immune effector function includes any one or more of increased recognition of an antigen or an antigen peptide derived from an antigen in the context of MHC class II molecules by T-cell receptors, increased release of cytokines and/or the activation of CD4⁺ lymphocytes, increased release of cytokines and/or the activation of CD8⁺ lymphocytes (CTLs) and/or B-cells, increased recognition of an antigen or an antigen peptide derived from an antigen in the context of MHC class I molecules by T-cell receptors, increased elimination of cells presented in the context of MHC class I molecules, i.e., cells characterized by presentation of an antigen with class I MHC, for example, via apoptosis or perforin-mediated cell lysis, increased production of cytokines such as IFN-γ and TNF-α, and increased specific cytolytic killing of antigen expressing target cells. Suitably, the immune effector cell has aberrant expression of nuclear LSD. In representative examples of this type, the immune effector expresses nuclear LSD at a higher level than the level than in a control [symbol] cell (e.g., an immune effector cells with normal or non-re [symbol] ector function).

In yet another aspect, the present invention provides methods of treating a T-cell dysfunctional disorder in a subject. These methods generally comprise, consist or consist essentially of administering concurrently to the subject a LSD inhibitor (e.g., a LSD1 inhibitor) and a PD-1 binding antagonist in effective amounts to treat the T-cell dysfunctional disorder. Suitably, the LSD inhibitor and PD-1 binding antagonist are administered in synergistically effective amounts. In some embodiments, the T-cell dysfunctional disorder is a disorder or condition of T-cells characterized by decreased responsiveness to antigenic stimulation and/or increased inhibitory signal transduction through PD-1. In some of the same and other embodiments, the T-cell dysfunctional disorder is one in which the T-cells have decreased ability to secrete cytokines, proliferate, or execute cytolytic activity. In illustrative examples of this type, the decreased responsiveness to antigenic stimulation results in ineffective control of a pathogen or tumor. In some embodiments, the T-cell dysfunctional disorder is one in which T-cells are anergic. Representative examples of T-cell dysfunctional disorders include unresolved acute infection, chronic infection and tumor immunity. In preferred embodiments, the T-cell dysfunctional disorder is a cancer or infection that comprises a T-cell (e.g., a CD8⁺ or CD4⁺ T-cell) with a mesenchymal phenotype. In representative examples of this type, the T-cell expresses nuclear LSD at a higher level than the level of expression of TBET in the same T-cell, and/or at a higher level than in an activated T-cell. In some of the same and other embodiments, the T-cell is one exhibiting T-cell exhaustion or anergy. In non-limiting examples of this type, the T-cell expresses a higher level of EOMES than TBET and/or has elevated expression of PD-1. In some embodiments, the T-cell is a tumor-infiltrating lymphocyte. In other embodiments, the T-cell is a circulating lymphocyte. In some embodiments, the cancer is skin cancer (e.g., melanoma), lung cancer, breast cancer, ovarian cancer, gastric cancer, bladder cancer, pancreatic cancer, endometrial cancer, colon cancer, kidney cancer, esophageal cancer, prostate cancer, colorectal cancer, glioblastoma, neuroblastoma, or hepatocellular carcinoma. In preferred embodiments, the cancer is a metastatic cancer. Preferably, the metastatic cancer is metastatic melanoma or metastatic lung cancer. In some embodiments, the methods further comprise further administering concurrently to the subject, with the LSD inhibitor (e.g., a LSD1 inhibitor) and the PD-1 binding antagonist, an ancillary agent (e.g., a chemotherapeutic agent) or ancillary therapy (e.g., ablation or cytotoxic therapy) for treating, or for aiding in the treatment of, a T-cell dysfunctional disorder. Preferably, the methods comprise further administering concurrently to the subject, with the LSD inhibitor (e.g., a LSD1 inhibitor) and the PD-1 binding antagonist, a chemotherapeutic agent, which suitably targets rapidly dividing cells and/or disrupt the cell cycle or cell division (e.g., a cytotoxic compound such as a taxane).

In related aspects, the present invention provides methods of treating or delaying the progression of cancer in a subject. These methods generally comprise, consist or consist essentially of administering concurrently to the subject a LSD inhibitor (e.g., a LSD1 inhibitor) and a PD-1 binding antagonist in effective amounts to treat or delay the progression of the cancer. In some embodiments, the subject has been diagnosed with cancer, wherein a T-cell in a tumor sample of the cancer from the subject expresses nuclear LSD at a higher level than the level of expression of TBET in the same T-cell, and/or at a higher level than in an activated T-cell.

In other related aspects, the present invention provides methods of enhancing immune function (e.g., immune effector function) in an individual having cancer. These methods [symbol] se, consist or consist essentially of administering concurre [symbol] LSD inhibitor (e.g., a LSD1 inhibitor) and a PD-1 binding antagonist in effective amounts to enhance the immune function. In some embodiments, the individual has been diagnosed with cancer, wherein a T-cell in a tumor sample of the cancer taken from the individual expresses nuclear LSD at a higher level than the level of expression of TBET in the same T-cell, and/or at a higher level than in an activated T-cell.

In further aspects, provided herein are methods of treating infection (e.g., with a bacteria or virus or other pathogen). These methods generally comprise, consist or consist essentially of administering concurrently to the individual a LSD inhibitor (e.g., a LSD1 inhibitor) and a PD-1 binding antagonist in effective amounts to treat the infection. In some embodiments, the infection is with virus and/or bacteria. In some embodiments, the infection is with a pathogen. In some embodiments, the infection is an acute infection. In some embodiments, the infection is a chronic infection.

In other related aspects, the present invention provides methods of enhancing immune function (e.g., immune effector function, T-cell function etc.) in an individual having an infection. These methods generally comprise, consist or consist essentially of administering concurrently to the individual a LSD inhibitor (e.g., a LSD1 inhibitor) and a PD-1 binding antagonist in effective amounts to enhance the immune function. In some embodiments, the individual has been diagnosed with the infection, wherein a T-cell in a sample taken from the individual expresses nuclear LSD at a higher level than the level of expression of TBET in the same T-cell, and/or at a higher level than in an activated T-cell.

Another aspect of the present invention provides use of a LSD inhibitor (e.g., a LSD1 inhibitor) and a PD-1 binding antagonist for treating a T-cell dysfunctional disorder, or for enhancing immune function (e.g., immune effector function, T-cell function etc.) in an individual having cancer, for treating or delaying the progression of cancer, or for treating infection. The LSD inhibitor and PD-1 binding antagonist are generally used in the manufacture of medicaments for this purpose. Suitably, the LSD inhibitor and PD-1 binding antagonist are formulated for concurrent administration.

In a related aspect, the present invention provides use of a LSD inhibitor (e.g., a LSD1 inhibitor), a PD-1 binding antagonist and an ancillary agent (e.g., a chemotherapeutic agent) for treating, or for aiding in the treatment of, a T-cell dysfunctional disorder, or for enhancing immune function (e.g., immune effector function, T-cell function etc.) in an individual having cancer, for treating or delaying the progression of cancer, or for treating infection. The LSD inhibitor, PD-1 binding antagonist and ancillary agent (e.g., a chemotherapeutic agent) are typically used in the manufacture of medicaments for this purpose. Suitably, the LSD inhibitor, PD-1 binding antagonist and ancillary agent (e.g., a chemotherapeutic agent) are formulated for concurrent administration. Preferably, the ancillary agent is a chemotherapeutic agent, which suitably targets rapidly dividing cells and/or disrupt the cell cycle or cell division (e.g., a cytotoxic compound such as a taxane).

In some embodiments, the methods for treating a T-cell dysfunctional disorder, or for enhancing immune function (e.g., immune effector function, T-cell function etc.) in an individual having cancer, for treating or delaying the progression of cancer, or for treating infection comprise detecting an elevated level of nuclear LSD (i.e., a LSD, suitably LSD1p, localized in the nucleus) in [symbol] ative to the level of TBET in the same T-cell or the level o [symbol] activated T-cell) in a sample obtained from the subject, prior to the concurrent administration.

In some embodiments, the methods for treating a T-cell dysfunctional disorder comprise detecting an elevated level of nuclear LSD (i.e., a LSD, suitably LSD1p, localized in the nucleus) in a T cell (e.g., relative to the level of TBET in the same T-cell or the level of nuclear LSD in an activated T-cell) and an elevated level of EOMES in the nucleus of the T cell (e.g., relative to the level of TBET in the same T-cell or the level of EOMES in the nucleus of an activated T-cell) in a sample obtained from the subject, prior to the concurrent administration. In representative examples of this type, these methods comprise detecting an elevated level of a complex comprising a LSD (e.g., a LSD1, suitably LSD1p) and EOMES, suitably in the nucleus of the T-cell.

In related aspects, the present invention provides kits comprising a medicament comprising a LSD inhibitor (e.g., a LSD1 inhibitor) and an optional pharmaceutically acceptable carrier, and a package insert comprising instructional material for concurrent administration of the medicament with another medicament comprising a PD-1 binding antagonist and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual. In some embodiments, the package insert comprises instructional material for concurrent administration of the medicament with another medicament comprising an ancillary agent and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual. Preferably, the ancillary agent is a chemotherapeutic agent, which suitably targets rapidly dividing cells and/or disrupt the cell cycle or cell division (e.g., a cytotoxic compound such as a taxane).

In other related aspects, the present invention provides kits comprising a medicament comprising a PD-1 binding antagonist and an optional pharmaceutically acceptable carrier, and a package insert comprising instructional material for concurrent administration of the medicament with another medicament comprising a LSD inhibitor (e.g., a LSD1 inhibitor) and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual. In some embodiments, the package insert comprises instructional material for concurrent administration of the medicament with another medicament comprising an ancillary agent and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual. Preferably, the ancillary agent is a chemotherapeutic agent, which suitably targets rapidly dividing cells and/or disrupt the cell cycle or cell division (e.g., a cytotoxic compound such as a taxane).

In still other related aspects, the present invention provides kits comprising a first medicament comprising a LSD inhibitor (e.g., a LSD1 inhibitor) and an optional pharmaceutically acceptable carrier, and a second medicament comprising a PD-1 binding antagonist and an optional pharmaceutically acceptable carrier for treating a T-cell dysfunctional disorder, or for enhancing [symbol] (e.g., immune effector function, T-cell function etc.) in a [symbol] cancer, for treating or delaying the progression of cancer, or for treating infection in an individual. In some embodiments, the kits further comprise a package insert comprising instructional material for administering concurrently the first medicament and the second medicament for treating a T-cell dysfunctional disorder, or for enhancing immune function (e.g., immune effector function, T-cell function etc.) in an individual having cancer, for treating or delaying the progression of cancer, or for treating infection in an individual. In some embodiments, the package insert comprises instructional material for concurrent administration of the medicament with another medicament comprising an ancillary agent and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual. Preferably, the ancillary agent is a chemotherapeutic agent, which suitably targets rapidly dividing cells and/or disrupt the cell cycle or cell division (e.g., a cytotoxic compound such as a taxane).

In further related aspects, the present invention provides kits comprising a first medicament comprising a LSD inhibitor (e.g., a LSD1 inhibitor) and an optional pharmaceutically acceptable carrier, a second medicament comprising a PD-1 binding antagonist and an optional pharmaceutically acceptable carrier and a third medicament comprising a chemotherapeutic agent and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual. In some embodiments, the kits further comprise a package insert comprising instructional material for administering concurrently the first medicament, second and third medicaments for treating a T-cell dysfunctional disorder, or for enhancing immune function (e.g., immune effector function, T-cell function etc.) in an individual having cancer, for treating or delaying the progression of cancer, or for treating infection in an individual. Preferably, the chemotherapeutic agent targets rapidly dividing cells and/or disrupt the cell cycle or cell division (e.g., a cytotoxic compound such as a taxane).

In some embodiments of the methods, uses, compositions, formulations and kits described above and elsewhere herein, CD8⁺ T cells in the individual have enhanced priming, activation, proliferation and/or cytolytic activity as compared to before the administration of the combination. In some embodiments, the number of CD8⁺ T cells is elevated as compared to before administration of the combination. In some embodiments, the CD8⁺ T cell is an antigen-specific CD8⁺ T cell. In some embodiments, Treg function is suppressed as compared to before administration of the combination of the LSD inhibitor (e.g., a LSD1 inhibitor) and PD-1 binding antagonist. In some embodiments, T cell exhaustion is decreased as compared to before administration of the combination of LSD inhibitor (e.g., a LSD1 inhibitor) and PD-1 binding antagonist. In some embodiments, number of Treg cells is decreased as compared to before administration of the combination of the LSD inhibitor (e.g., a LSD1 inhibitor) and PD-1 binding antagonist. In some embodiments, plasma IFN-γ is increased as compared to before administration of the combination of the LSD inhibitor (e.g., a LSD1 inhibitor) and PD-1 binding antagonist. In some embodiments, plasma TNF-α is increased as compared to before administration of the combination of the LSD inhibitor (e.g., a LSD1 inhibitor) and PD-1 binding antagonist. In some embodiments, the number of memory T effector cells is increased as compared to before administration of the combination of LSD inhibitor (e.g., a LSD1 inhibitor) and PD-1 binding [symbol] me embodiments, memory T effector cell activation and/ [symbol] increased as compared to before administration of the combination of the LSD inhibitor (e.g., a LSD1 inhibitor) and PD-1 binding antagonist. In some embodiments, memory T effector cells are detected in peripheral blood. In some embodiments, detection of memory T effector cells is by detection of CXCR3.

In some embodiments of the methods, uses, formulations, and kits described above and elsewhere herein, the LSD inhibitor (e.g., a LSD1 inhibitor) and PD-1 binding antagonist are administered intravenously, intramuscularly, subcutaneously, topically, orally, transdermally, intraperitoneally, intraorbitally, by implantation, by inhalation, intrathecally, intraventricularly, or intranasally. In some embodiments of the methods, uses, compositions, and kits described above and herein, the treatment further comprises administering an ancillary agent (e.g., a chemotherapeutic agent) for treating or delaying progression of cancer in an individual. In some embodiments, the individual has been treated with a chemotherapeutic agent (e.g., a compound that targets rapidly dividing cells and/or disrupt the cell cycle or cell division, suitably a cytotoxic compound such as a taxane) before the combination treatment with the LSD inhibitor and PD-1 binding antagonist. In some embodiments, the individual treated is refractory to a chemotherapeutic agent treatment. Some embodiments of the methods, uses, compositions, and kits described throughout the application, further comprise administering a chemotherapeutic agent for treating or delaying progression of cancer.

A further aspect of the present invention provides methods of diagnosing the presence of a T-cell dysfunctional disorder in a subject. These methods generally comprise, consist or consist essentially of:
(i) obtaining a sample from the subject, wherein the sample comprises a T-cell (e.g., CD8⁺ T-cell or CD4⁺ T-cell);
(ii) contacting the sample with a first binding agent that binds to a LSD (e.g., a nuclear LSD such as LSD1p) in the sample and a second binding agent that binds to EOMES in the sample; and
(iii) detecting localization of the first and second binding agents in the nucleus of the T-cell;
   wherein localization of the first and second binding agents in the nucleus of the T-cell is indicative of the presence of the T-cell dysfunctional disorder in the subject.

In yet another aspect, the present invention provides methods of diagnosing the presence of a T-cell dysfunctional disorder in a subject. These methods generally comprise, consist or consist essentially of:
(i) obtaining a sample from the subject, wherein the sample comprises a T-cell (e.g., CD8⁺ T-cell or CD4⁺ T-cell);
(ii) contacting the sample with a first binding agent that binds to a LSD (e.g., a nuclear LSD such as LSD1p) in the sample and a second binding agent that binds to EOMES in the sample; and
(iii) detecting the first and second binding agents when bound to a LSD-EOMES complex in the sample;
   wherein an elevated level of LSD-EOMES complex detected in the sample relative to a level of LSD-EOMES complex detected in a control sample (e.g., one comprising an activated T-cell) is indicative of the presence of the T-cell dysfunctional disorder in the subject.

[symbol] further aspect of the present invention provides method [symbol] treatment of a subject with a T-cell dysfunctional disorder. These methods generally comprise, consist or consist essentially of:
(i) obtaining a sample from the subject following treatment of the subject with a therapy for the T-cell dysfunctional disorder, wherein the sample comprises a T-cell (e.g., CD8⁺ T-cell or CD4⁺ T-cell);
(ii) contacting the sample with a first binding agent that binds to a LSD (e.g., a nuclear LSD such as LSD1p) in the sample and a second binding agent that binds to EOMES in the sample; and
(iii) detecting the first and second binding agents when bound to a LSD-EOMES complex in the sample;
   wherein a lower level of LSD-EOMES complex detected in the sample relative to a level of LSD EOMES complex detected in a control sample taken from the subject prior to the treatment is indicative of an increased clinical benefit (e.g., enhanced immune effector function such as enhanced T-cell function) to the subject, and
   wherein a higher level of LSD-EOMES complex detected in the sample relative to a level of LSD EOMES complex detected in a control sample taken from the subject prior to the treatment is indicative of no or negligible clinical benefit (e.g., enhanced immune effector function such as enhanced T-cell function) to the subject.

In still another aspect, a kit is provided for diagnosing the presence of a T-cell dysfunctional disorder in a subject. These kits generally comprise, consist or consist essentially of: (i) a first binding agent that binds to a LSD (e.g., a nuclear LSD such as LSD1p), (ii) a second binding agent that binds to EOMES; and (iii) a third agent comprising a label, which is detectable when each of the first and second binding agents is bound to a LSD-EOMES complex. In specific embodiments, the third agent is a binding agent that binds to the first and second binding agent.

In a related aspect, the present invention provides a complex comprising a LSD (e.g., a nuclear LSD such as LSD1p) and EOMES, a first binding agent that is bound to the LSD of the complex, a second binding agent bound to EOMES of the complex; and (iii) a third agent comprising a label, which is detectable when each of the first and second binding agents is bound to the LSD-EOMES complex. In specific embodiments, the LSD-EOMES complex is located in a T-cell, suitably in the nucleus of the T-cell. In specific embodiments, the third agent is a binding agent that binds to the first and second binding agent.

In still another aspect, the present invention provides a T-cell that comprises a complex comprising a LSD (e.g., a nuclear LSD such as LSD1p) and EOMES, a first binding agent that is bound to the LSD of the complex, a second binding agent bound to EOMES of the complex; and (iii) a third agent comprising a label, which is detectable when each of the first and second binding agents is bound to the LSD-EOMES complex. In specific embodiments, the third agent is a binding agent that binds to the first and second binding agent.

In any of the above aspect, respective binding agents are preferably antibodies.

The above diagnostic methods and kits are useful as companion diagnostics for the treatment methods of the invention.

### BRIEF DESCRIPTION OF THE DRAWINGS

FIG. 1 is a graphical representation showing efficacy of the LSD1 inhibitor, phenelzine sulfate in inhibiting demethylation and cell proliferation of a breast cancer cell line. A. MDA-MB-231 breast cancer cell line was treated with increasing doses of phenelzine sulfate (phenelzine). Immunofluorescence microscopy was performed on cells fixed and probed with primary anti-LSD1s111p and anti-H3k4me2 and DAPI. Representative images for each dataset are shown. Graph represents the TNFI values for LSD1p and H3k4me2 measured using ImageJ to select the nucleus minus background (n>20 individual cells). B. The effect of phenelzine sulfate on cell proliferation of MDA-MB-231 cells was analyzed using the WST-1 proliferation assay

FIG. 2 is a graphical and photographic representation showing the efficacy of dual phenelzine sulfate (phenelzine) and anti-PD1 antibody (PD1) therapy on circulating tumor cells (CTCs) and cancer stem cells (CSC) as well as tumor burden. A. Statistics of tumor volumes on day 15 post-treatment. A Mann-Whitney non-parametric t-test was used to compare control vs other groups (*p<0.02, **p<0.008). Group A=Control, Group C: PD1 (10 mg/kg), Group D: Phenelzine (40 mg/kg), Group F: PD1+Phenelzine. Illustration of the treatment protocol is also shown. B. Immunofluorescence microscopy was performed on cells fixed and probed with primary anti-LSD1, anti-Snail and anti CSV antibodies and DAPI. Representative images for each dataset are shown. Graph represents the TNFI values for LSD1, SNAIL and TCFI for CSV measured using ImageJ to select the nucleus minus background (n>20 individual cells, Group A=Control, Group C: PD1 (10 mg/kg), Group D: Phenelzine (40 mg/kg), Group F: PD1+Phenelzine). C. Immunofluorescence microscopy was performed on cells fixed and probed with primary anti-CD133, anti-ALDH1A and anti ABCB5 antibodies and DAPI. (a) Representative images for each dataset are shown. (b) Graph represents the TCFI values for CD133 TNFI for ALDH1A and TFI for ABCB5 measured using ImageJ to select the nucleus/cytoplasm minus background (n>20 individual cells, Group A=Control, Group C: PD1 (10 mg/kg), Group D: Phenelzine (40 mg/kg), Group F: PD1+Phenelzine).

FIG. 3 is a photographic and graphical representation showing that dual epigenetic-immunotherapy inhibits metastatic progression in 4T1 Mouse model. A. 4T1 Treatment FFPE from each treatment group (Group A=Control, Group C: PD1 (10 mg/kg), Group D: Phenelzine (40 mg/kg), Group F: PD1+Phenelzine) and for each target organ (Lung and Liver) was processed for 3D high resolution microscopy using the BondRX. FFPE tissues were fixed and Immunofluorescence microscopy was performed probing with rabbit LSD1(S111p); mouse anti CSV and goat anti SNAIL and visualized with a donkey anti-rabbit AF 488, anti-mouse 568 and anti-goat 633. Cover slips were mounted on glass microscope slides with ProLong Diamond Antifade reagent (Life Technologies). Protein targets were localized by confocal laser scanning microscopy. Single 0.5 μm sections were obtained using a Leica DMI8 microscope using 100× oil immersion lens running LAX software. The final image was obtained by averaging four sequential images of the same section. Digital images were analyzed using ImageJ software (ImageJ, NIH, Bethesda, Md., USA) to determine the either the Total Nuclear Fluorescent Intensity (TNFI), the Total Cytoplasmic Fluorescent Intensity (TCFI) or total Fluorescent Intensity (TFI). N=40 cells were counted. B. Graphs for both organs are presented here. Graphs represent the TNFI values for LSD1, ALDH1A and TCFI for CSV measured using ImageJ to select the nucleus minus background. Representative images for the Lung dataset are shown.

FIG. 4 is a graphical representation showing that dual epigenetic-immunotherapy re-educates and re-programs innate macrophage repertoire. A. Immunofluorescence microscopy was performed on F4/80+MØ cells fixed and probed with a M1 panel consisting of anti-CD38 and [symbol] odies and DAPI. Graph represents the TFI for CD38 and T [symbol] ured using ImageJ to select the nucleus minus background (n>20 individual cells, Group A=Control, Group C: PD1 (10 mg/kg), Group D: Phenelzine (40 mg/kg), Group F: PD1+Phenelzine). B. Immunofluorescence microscopy was performed on F4/80+MØ cells fixed and probed with a M2 panel consisting of anti-CD206 and anti LSD1p antibodies and DAPI. Graph represents the TFI for CD206 and TNFI for LSD1p measured using ImageJ to select the nucleus minus background (n>20 individual cells, Group A=Control, Group C: PD1 (10 mg/kg), Group D: Phenelzine (40 mg/kg), Group F: PD1+Phenelzine).

FIG. 5 is a graphical representation showing that dual epigenetic-immunotherapy re-educates and re-programs the T-cell repertoire. A. Cells stimulated with PMA/ionomycin for 4 hours in the presence of Brefeldin A. Cells were surfaced stained with CD45, CD3, CD4, CD8, CD44, CD62L (for naïve, effector and central memory), CD25 and FoxP3 (for Tregs) and analyzed via Flow cytometry. A Mann-Whitney non-parametric t-test was used to compare control vs other groups (*p<0.05, n=2-5). B. Immunofluorescence microscopy was performed on cells fixed and probed with primary anti-CD8, anti-EOMES and anti TBET antibodies and DAPI. Representative images for each dataset are shown. TNFI values for EOMES, TBET and Ki67 measured using Image) to select the nucleus minus background. Graphs represent the % change of expression relative to control group for CD8⁺ T-cells. (n>20 individual cells, Group A=Control, Group C: PD1 (10 mg/kg), Group D: Phenelzine (40 mg/kg), Group F: PD1+Phenelzine). C. Cells stimulated with PMA/ionomycin for 4 hours in the presence of Brefeldin A. Cell were surfaced stained with CD45, CD3, CD4, CD8 and intracellular staining of IFN-γ, IL-2 and TNF-α in the presence of Brefeldin A and analyzed via flow cytometry. A Mann-Whitney non-parametric t-test was used to compare control vs other groups (*p<0.05, **p<0.008; n=2-5). D. Immunofluorescence microscopy was performed on cells fixed and probed with primary anti-CD8, anti-TNFα and anti IFNγ antibodies and DAPI. Representative images for each dataset are shown. TNFI values for TNFα and IFNγ measured using ImageJ to select the nucleus minus background. Graphs represent the % change of expression relative to control group for CD8⁺ T-cells. (n>20 individual cells, Group A=Control, Group C: PD1 (10 mg/kg), Group D: Phenelzine (40 mg/kg), Group F: PD1+Phenelzine). E. Nanostring analysis was performed on CD8+−T-cells isolated from the TME of the 4T1 metastatic mouse model (Group A=Control, Group C: PD1 (10 mg/kg), Group D: Phenelzine (40 mg/kg), Group F: PD1+Phenelzine) analyzing the expression of T-Cell activation markers.

FIG. 6 is a graphical and photographic representation depicting nuclear LSD1 complexes with EOMES in exhausted T-cell signatures. A. Nanostring transcript analysis was performed on isolated CD8⁺ T-cells isolated from a 4T1 metastatic cancer mouse model treated with either Group A=Control, Group C: PD1 (10 mg/kg), Group D: Phenelzine (40 mg/kg), or Group F: PD1+Phenelzine. Displayed are the effects on expression of mRNA of genes associated with exhaustion or activation T-cell markers. B. Immunofluorescence microscopy was performed on CD8⁺ T-cells fixed and probed with primary anti-LSD1, anti-EOMES and anti-CD8 antibodies and DAPI. Representative images for each dataset are shown. Graph represents the TNFI values for LSD1 and EOMES were measured using ImageJ to select the nucleus minus background (n>20 individual cells, Group A=Control, Group C: PD1 (10 mg/kg), Group D: Phenelzine (40 mg/kg), Group F: PD1+Phenelzine). C. Displays plot-profiles for EOMES/LSD1. Plot-profiles were plotted with the use of ImageJ software measuring a series of fluorescent intensities along a line spanning the nucleus. The pattern of the two plots can give an insight into the nature of the relationship between the two fluorochromes. Imagej software with automatic thresholding and manual selection [symbol] for nucleus of cells was used to calculate the Pearson's co [symbol] (PCC) for each pair of antibodies. The PCC Values range from −1=inverse of co-localization, 0=no co-localization, +1=perfect co-localization.

FIG. 7 is a photographic and graphical representation showing that nuclear LSD1 expression coincides with TBET repression in exhausted T-cell signatures. Immunofluorescence microscopy was performed on CD8⁺ T-cells fixed and probed with primary anti-LSD1, anti-TBET and anti CD8 antibodies and DAPI. Representative images for each dataset are shown. Graph represents the TNFI values for LSD1 and TBET were measured using ImageJ to select the nucleus minus background (n>20 individual cells, Group A=Control, Group C: PD1 (10 mg/kg), Group D: Phenelzine (40 mg/kg), Group F: PD1+Phenelzine). Imagej software with automatic thresholding and manual selection of ROI's specific for nucleus of cells was used to calculate the Pearson's co-efficient correlation (PCC) for each pair of antibodies. The PCC Values range from −1=inverse of co-localization, 0=no co-localization, +1=perfect co-localization.

FIG. 8 is a graphical representation showing that dual epigenetic-immunotherapy re-programs gene expression programs in CD8⁺ T-cells. A. Nanostring analysis is shown of the three treatment groups effect on the mRNA expression of genes either 2 or 3 fold higher or 2 or 3 fold lower relative to control group A (the groups are: C: PD1 (10 mg/kg), Group D: Phenelzine (40 mg/kg), Group F: PD1+Phenelzine). Plotted is the overlap between the 3 groups as well as the genes specifically effected by each individual treatment group or genes specifically induced or abrogated by the combination treatment group (Group F). This is in purified CD8⁺ T-cells the 4T1 metastatic mouse model. B. % genes affected (cut-off of 2 fold or greater than 2 fold or lower) is shown for each of the listed gene pathways and for each treatment group or combination. This is in purified CD8⁺ T-cells the 4T1 metastatic mouse model. C. This figure depicts the effect of expression or inhibition on the adaptive, innate, inflammation, cancer progression and T-cell function message by treatment with phenelzine, PD1 or combination in purified CD8⁺ T-cells in the 4T1 metastatic mouse model. D. ATAQ Sequencing showing chromatin accessibility changes directly mediated by LSD1 in purified CD8⁺ T-cells from the 4T1 metastatic mouse model.

FIG. 9 is a photographic and graphical representation showing that EOMES and LSD1 form a complex in exhausted CD8⁺ T-Cells. A. The DUOLINK immunofluorescence ligation assay was performed on CD8⁺ T-cells fixed and probed with primary anti-LSD1, anti-EOMES and anti CD8 antibodies and DAPI. Representative images for each dataset are shown Red dots/staining represents the interaction of EOMES and LSD1. Graph represents the TNFI values for the complex of EOMES and LSD1 and were measured using ImageJ to select the nucleus minus background (n>20 individual cells, Group A=Control, Group C: PD1 (10 mg/kg), Group D: Phenelzine (40 mg/kg), Group F: PD1+Phenelzine). B. The DUOLINK Immunofluorescence ligation assay was performed on CD8⁺ T-cells isolated from QR (immune Reactive) patients with stable anti-CMV T-cell immunity and QNR (non-reactive) without anti-CMV T-cell immunity (measured by IFN-CMV-specific IFN-γ secretion). Red dots/staining represents the interaction of EOMES and LSD1.

FIG. 10 presents the amino acid sequence of human EOMES. The red bold text is a predicted monopartite nuclear localization sequence (NLS), the text highlighted in yellow (with the lysine target in bold red) represents potential methylation sites near the sequence of the NLS with a support vector machine (SVM) probability of ˜0.7 or higher. It is predicted that based on these 3 putative sites near the NLS (prediction 0.81, 0.7, 0.93) as well as a 4th putative methylation site (lysine) (RQEISFGKLKLTNNKGANN) in the middle of the NLS sequence, there is a high [symbol] SD1 regulates EOMES via demethylation of these sites po [symbol] nuclear localization.

FIG. 11 is a photographic and graphical representation showing triple therapy efficacy on CTC/CSC and tumor burden. A. Statistics of tumor volumes on day 15 post-treatment. A Mann-Whitney non-parametric t-test was used to compare control vs other groups (*p<0.02, **p<0.008). Group A=Control, Group B: Abraxane (30 mg/kg), Group C: PD1 (10 mg/kg), Group D: Phenelzine (40 mg/kg), Group E: Abraxane+PD1, Group F: PD1+Phenelzine, Group G: Abraxane+Phenelzine, Group H: Triple Therapy (i.e., Abraxane+Phenelzine+PD1 antibody). Illustration of the treatment protocol is also shown. B. Key for treatment groups for Groups A to H. C. Illustration of the treatment protocol. D. Immunofluorescence microscopy was performed on cells fixed and probed with primary anti-LSD1, anti-Snail and anti CSV antibodies and DAPI. Representative images for each dataset are shown. Graph represents the TNFI values for LSD1, SNAIL and TCFI for CSV measured using ImageJ to select the nucleus minus background (n>20 individual cells, Group A=Control, Group B: Abraxane (30 mg/kg), Group C: PD1 (10 mg/kg), Group D: Phenelzine (40 mg/kg), Group E: Abraxane+PD1, Group F: PD1+Phenelzine, Group G: Abraxane+Phenelzine, Group H: Triple Therapy). E. Immunofluorescence microscopy was performed on cells fixed and probed with primary anti-CD133, anti-ALDH1A and anti ABCB5 antibodies and DAPI. (a) Representative images for each dataset are shown. (b) Graph represents the TCFI values for CD133 TNFI for ALDH1A and TFI for ABCB5 measured using ImageJ to select the nucleus/cytoplasm minus background (n>20 individual cells, Group A=Control, Group B: Abraxane (30 mg/kg), Group C: anti-PD1 (10 mg/kg), Group D: Phenelzine (40 mg/kg), Group E: Abraxane+PD1, Group F: PD1+Phenelzine, Group G: Abraxane+Phenelzine, Group H: Triple Therapy).

Some figures and text contain color representations or entities. Color illustrations are available from the Applicant upon request or from an appropriate Patent Office. A fee may be imposed if obtained from a Patent Office.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter per se.

“Activation”, as used herein, refers to the state of a cell following sufficient cell surface moiety ligation to induce a noticeable biochemical or morphological change. Within the context of T cells, such activation refers to the state of a T cell that has been sufficiently stimulated [symbol] proliferation. Activation of a T cell may also induce cytok [symbol] detectable effector functions, including performance of regulatory or cytolytic effector functions. Within the context of other cells, this term infers either up or down regulation of a particular physico-chemical process. Activation can also be associated with induced cytokine production, and detectable effector functions.

The term “activated T-cell” means a T-cell that is currently undergoing cell division, detectable effector functions, including cytokine production, performance of regulatory or cytolytic effector functions, and/or has recently undergone the process of “activation”.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

“Amplification,” as used herein generally refers to the process of producing multiple copies of a desired sequence. “Multiple copies” mean at least two copies. A “copy” does not [symbol] perfect sequence complementarity or identity to the tem [symbol] example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The “amount” or “level” of a biomarker is a detectable level in a sample. These can be measured by methods known to one skilled in the art and also disclosed herein. The expression level or amount of biomarker assessed can be used to determine the response to treatment.

As used herein, “and/or” refers to and encompasses any and all possible combinations of one or more of the associated listed items, as well as the lack of combinations when interpreted in the alternative (or).

The term “anergy” refers to the state of unresponsiveness to antigen stimulation resulting from incomplete or insufficient signals delivered through the T-cell receptor (e.g. increase in intracellular Ca²⁺ in the absence of ras-activation). T-cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T-cells do not undergo clonal expansion and/or acquire effector functions.

The term “antagonist” or “inhibitor” refers to a substance that prevents, blocks, inhibits, neutralizes, or reduces a biological activity or effect of another molecule, such as an enzyme or receptor. The term “specific antagonist” or “specific inhibitor” refers to a compound with high specificity for its target (e.g. for a LSD such as LSD1 including nuclear LSD, or for PD-1). Specificity of a particular antagonist or inhibitor is defined as a ratio of the IC50 values of the particular antagonist or inhibitor for the target of interest versus another target. For example, an antagonist that is specific for PD-1 will have an IC50 value for target A (e.g., PD-1) lower than that for target B (e.g., PD-L1 or PD-L2). Likewise, an inhibitor that is specific for LSD1 will have an IC50 value for target A (e.g., LSD1) lower than that for target B (e.g., LSD2). For example, the IC50 value for target A is at least 10 times lower than the IC50 value of the same inhibitor for target B. In other examples, the IC50 value for target A is 100 times lower, or in other example is 1000 times lower. In still other example, the IC50 value for target A is 10,000 times lower than the IC50 value of the same inhibitor for target B. The term “specific” is used interchangeably herein with the term “selective”. In certain embodiments, the term “selective” is used herein to refer to compounds that inhibit or display antagonism towards a LSD without displaying substantial inhibition or antagonism towards another LSD or another enzyme such as a monoamine oxidase (MAO) (e.g., MAO A or MAO B). Accordingly, a compound that is selective for LSD1 exhibits a LSD1 selectivity of greater than about 2-fold, 5-fold, 10-fold, 20-fold, 50-fold or greater than about 100-fold with respect to inhibition or antagonism of another LSD (i.e., a LSD other than LSD1 such as LSD2) or of another enzyme (e.g., a MAO). In some embodiments, selective compounds display at least 50-fold greater inhibition or antagonism towards a specified LSD than towards another LSD or another enzyme (e.g., a MAO). In still other embodiments, selective compounds inhibit or display at least 100-fold greater inhibition or antagonism towards a specified LSD than towards another LSD or another enzyme (e.g., a MAO). In still other embodiments, selective compounds display at least 500-fold greater inhibition or antagonism towards L a specified LSD than towards another LSD or another enzyme (e.g., a MAO). In still other embodiments, selective compounds display at least [symbol] r inhibition or antagonism towards a specified LSD than t [symbol] another enzyme (e.g., a MAO).

The term “antagonist antibody” refers to an antibody that binds to a target and prevents or reduces the biological effect of that target. In some embodiments, the term can denote an antibody that prevents the target, e.g., PD-1, to which it is bound from performing a biological function.

As used herein, an “anti-PD-1 antagonist antibody” refers to an antibody that is able to inhibit PD-1 biological activity and/or downstream events(s) mediated by PD-1. Anti-PD-1 antagonist antibodies encompass antibodies that block, antagonize, suppress or reduce (to any degree including significantly) PD-1 biological activity, including inhibitory signal transduction through PD-1 and downstream events mediated by PD-1, such as PD-L1 binding and downstream signaling, PD-L2 binding and downstream signaling, inhibition of T cell proliferation, inhibition of T cell activation, inhibition of IFN secretion, inhibition of IL-2 secretion, inhibition of TNF secretion, induction of IL-10, and inhibition of anti-tumor immune responses. For purposes of the present invention, it will be explicitly understood that the term “anti-PD-1 antagonist antibody” (interchangeably termed “antagonist PD-1 antibody”, “antagonist anti-PD-1 antibody” or “PD-1 antagonist antibody”) encompasses all the previously identified terms, titles, and functional states and characteristics whereby PD-1 itself, a PD-1 biological activity, or the consequences of the biological activity, are substantially nullified, decreased, or neutralized in any meaningful degree. In some embodiments, an anti-PD-1 antagonist antibody binds PD-1 and upregulates an anti-tumor or anti-pathogen immune response. Examples of anti-PD-1 antagonist antibodies are provided herein.

The term “antibody” herein is used in the broadest sense and specifically covers monoclonal antibodies (including full length monoclonal antibodies), polyclonal antibodies, chimeric antibodies, humanized antibodies, human antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable [symbol] d (V_{L}) and a constant domain at its other end; the const [symbol] chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the C_{H1}, C_{H2} and C_{H3} domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“K”) and lambda (“A”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

[symbol] he terms “full length antibody,” “intact antibody” and “wh [symbol] herein interchangeably to refer to an antibody in its substantially intact form, not antibody fragments as defined below. The terms particularly refer to an antibody with heavy chains that contain an Fc region.

The term “naïve T-cells” refers to immune cells that comprise antigen-inexperienced cells, e.g., immune cells that are precursors of memory T effector cells. In some embodiments, nave T cells may be differentiated, but have not yet encountered their cognate antigen, and therefore are activated T cells or memory effector T cells. In some embodiments, nave T cells may be characterized by expression of CD62L, CD27, CCR7, CD45RA, CD28, and CD127, and the absence of CD95, or CD45RO isoform.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., [symbol] Pharmacology of Monoclonal Antibodies, vol. 113, Rosen [symbol] (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. [symbol] SA 90: 2551 (1993); Jakobovits et al., Nature 362: 255-PCT/AU2018/051268 Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to [symbol] ge, but whose endogenous loci have been disabled, e.g., [symbol] (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10⁻⁷ M, preferably no more than about 1×10⁻⁸ M and preferably no more than about 1×10⁻⁹ M) but has a binding affinity for a homologue of the antigen from a second nonhuman mammalian species which is at least about 50-fold, or at least about 500-fold, or at least about 1000-fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat Numbering) |
| H1 | H31-H35 | H26-H35 | H26-H32 | H30-H35 (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94- [symbol] H3) in the VH. The variable domain residues are numbere [symbol] et al., supra, for each of these definitions.

“Framework” or “FR” residues are those variable domain residues other than the HVR residues as herein defined. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The expression “linear antibodies” refers to the antibodies described in Zapata et al. (1995 Protein Eng, 8(10):1057-1062). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In [symbol] ents, an antibody that specifically binds to a target has a [symbol] (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

As used herein, the term “binding agent” refers to an agent that binds to a target antigen and does not significantly bind to unrelated compounds. Examples of binding agents that can be effectively employed in the disclosed methods include, but are not limited to, lectins, proteins, and antibodies, such as monoclonal antibodies, chimeric antibodies, or polyclonal antibodies, or antigen-binding fragments thereof, as well as aptamers, Fc domain fusion proteins, and aptamers having or fused to hydrophobic protein domain, e.g., Fc domain, etc. In an embodiment the binding agent is an exogenous antibody. An exogenous antibody is an antibody not naturally produced in a mammal, e.g. in a human, by the mammalian immune system.

The term “biomarker” as used herein refers to an indicator, e.g., predictive, diagnostic, and/or prognostic, which can be detected in a sample. The biomarker may serve as an indicator of a particular subtype of a disease or disorder (e.g., T-cell dysfunctional disorder) characterized by certain, molecular, pathological, histological, and/or clinical features. In some embodiments, a biomarker is a gene. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers.

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic. The biomarker signature may serve as an indicator of a particular subtype of a disease or disorder (e.g., T-cell dysfunctional disorder) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The terms “cancer” and “cancerous” refer to or describe the physiological condition in subjects that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic [symbol] carcinoma, penile carcinoma, melanoma, superficial sprea [symbol] tigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phacomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In specific embodiments, the cancer is melanoma or lung cancer, suitably metastatic melanoma or metastatic lung cancer.

The terms “cell proliferative disorder”, “proliferative disorder” and “hyperproliferative disorder” are used interchangeably herein to refer to disorders that are associated with some degree of abnormal cell proliferation. In some embodiments, the cell proliferative disorder is cancer. In some embodiments, the cell proliferative disorder is a tumor, including a solid tumor.

“Chemotherapeutic agent” includes compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; [symbol] sarcodictyin; spongistatin; nitrogen mustards such as ch [symbol] chlonnaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin y1I and calicheamicin con (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), [symbol] ozole, RIVISOR® (vorozole), FEMARA® (letrozole; Novar [symbol] (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-α, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzunnab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/3695, Wyeth Research and Abbott Laboratories) which is a recombinant exclusively human-sequence, full-length IgG₁λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-α for EGFR binding (EMD/Merck); human EGFR antibody, HuMax-EGFR (GenMab); fully human antibodies [symbol] 2.4, E2.5, E6.2, E6.4, E2.11, E6. 3 and E7.6. 3 and descr [symbol] U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoli-ne, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 aR)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described [symbol] wing patent publications: U.S. Pat. No. 5,804,396; WO 1 [symbol] Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor α (TNF-α) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T-cell costimulation blockers such as abatacept (Orencia), Interleukin 6 (IL-6) blockers such as tocilizumab (ACTEMERA®); Interleukin 13 (IL-13) blockers such as lebrikizumab; Interferon α (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin α (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², pb²¹² and radioactive isotopes of Lu); miscellaneous investigational agents such as thioplatin, PS-341, phenylbutyrate, ET-18-OCH 3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONELC)); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of [symbol] ; as well as combinations of two or more of the above su [symbol] abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

As used herein, a “companion diagnostic” refers to a diagnostic method and or reagent that is used to identify subjects susceptible to treatment with a particular treatment or to monitor treatment and/or to identify an effective dosage for a subject or sub-group or other group of subjects. For purposes herein, a companion diagnostic refers to reagents, such as a reagent for detecting, measuring or localizing a T-cell function biomarker (e.g., as described herein) in a sample. The companion diagnostic refers to the reagents and also to the test(s) that is/are performed with the reagent.

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). The term “polypeptide complex” or “protein complex,” as used herein, refers to a trimer, tetramer, pentamer, hexamer, heptamer, octamer, nonamer, decamer, undecamer, dodecamer, or higher order oligomer. In specific embodiments, the polypeptide complexes are formed by self-assembly of a LSD (e.g., a LSD1 such as LSD1p) and EOMES.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant [symbol] ments listed after the phrase, and limited to other eleme [symbol] ere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “correlate” or “correlating” refer to determining a relationship between one type of data with another or with a state (e.g., T-cell activation status, mesenchymal state, immune status, etc.). In some embodiments, “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

By “corresponds to” or “corresponding to” is meant an amino acid sequence that displays substantial sequence similarity or identity to a reference amino acid sequence. In general the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

As used herein, the term “cytolytic activity” refers to ability of a cell, e.g., a CD8+ cell or an NK cell, to lyse target cells. Such cytolytic activity can be measured using standard techniques, e.g., by radioactively labeling the target cells.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In some embodiments, the cytotoxic agent is a taxane. In representative examples of this type, the taxane is paclitaxel or docetaxel. In some embodiments, the cytotoxic agent is a platinum agent. In some embodiments, the cytotoxic agent is an antagonist of EGFR. In representative examples of this type, the antagonist of EGFR is N-(3-ethynylphenyl)-6,7-bis(2-methoxyethoxy)quinazolin-4-amine (e.g., erlotinib). In some embodiments, the cytotoxic agent is a RAF inhibitor. In non-limiting examples of this type, the RAF [symbol] F and/or CRAF inhibitor. In other non-limiting examples, [symbol] vemurafenib. In one embodiment the cytotoxic agent is a PI3K inhibitor.

As used herein, the term “cytotoxic therapy” refers to therapies that induce cellular damage including but not limited to radiation, chemotherapy, photodynamic therapy, radiofrequency ablation, anti-angiogenic therapy, and combinations thereof. A cytotoxic therapeutic may induce DNA damage when applied to a cell.

As used herein, “delaying progression of a disease” or “decreasing the rate of progression of a disease” means to defer, hinder, slow, retard, stabilize, and/or postpone development of the disease (such as a T-cell dysfunctional disorder). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

The term “detection” includes any means of detecting, including direct and indirect detection.

The term “diagnosis” is used herein to refer to the identification or classification of a molecular or pathological state, disease or condition (e.g., T-cell dysfunctional disorder). For example, “diagnosis” may refer to identification of a particular type of T-cell dysfunctional disorder. “Diagnosis” may also refer to the classification of a particular subtype of T-cell dysfunctional disorder, e.g., by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., T-cell dysfunctional disorder). For example, a method of aiding diagnosis of a disease or condition (e.g., T-cell dysfunctional disorder) can comprise measuring certain biomarkers in a biological sample from an individual.

A “disorder” is any condition that would benefit from treatment including, but not limited to, chronic and acute disorders or diseases including those pathological conditions which predispose a subject to the disorder in question.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into downstream T-cell effector functions, such as proliferation, cytokine production (e.g., IL-2, IFN-γ, TNF-α, etc.) and/or target cell killing.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically [symbol] For prophylactic use, beneficial or desired results include [symbol] eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the cancer or tumor. In the case of an infection, an effective amount of the drug may have the effect in reducing pathogen (bacterium, virus, etc.) titers in the circulation or tissue; reducing the number of pathogen infected cells; inhibiting (i.e., slow to some extent or desirably stop) pathogen infection of organs; inhibit (i.e., slow to some extent and desirably stop) pathogen growth; and/or relieving to some extent one or more of the symptoms associated with the infection. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer. A patient who “does not have an effective response” to treatment refers to a patient who does not have any one of extending survival (including overall survival and progression free survival); resulting in an objective response (including a complete response or a partial response); or improving signs or symptoms of cancer.

“Enhancing T-cell function” means to induce, cause or stimulate a T-cell to have a sustained or amplified biological function, or renew or reactivate exhausted or inactive T-cells. Examples of enhancing T-cell function include any one or more of: increased secretion of IFN-γ, increased secretion of TNF-α, increased secretion of IL-2 from CD8⁺ T-cells, increased proliferation, increased antigen responsiveness (e.g., viral, pathogen, or tumor clearance) relative to such levels before the intervention. In some embodiments, the level of enhancement is as least 50%, alternatively 60%, 70%, 80%, 90%, 100%, 120%, 150%, 200%. The manner of measuring this enhancement is known to one of ordinary skill in the art. [symbol] he term “epithelial phenotype” is understood in the art, a [symbol] by morphological, molecular and/or functional characteristics. For example, epithelial cells generally have a rounded or cobblestone appearance, express the epithelial marker E-cadherin, are rapidly dividing and/or have relatively low levels of motility, invasiveness and/or anchorage-independent growth as compared with mesenchymal cells.

As used herein, the term “epithelial-to-mesenchymal transition” (EMT) refers to the conversion from an epithelial to a mesenchymal phenotype, which is a normal process of embryonic development. EMT is also the process whereby injured epithelial cells that function as ion and fluid transporters become matrix remodeling mesenchymal cells. In carcinomas, this transformation typically results in altered cell morphology, the expression of mesenchymal proteins and increased invasiveness. The criteria for defining EMT in vitro involve the loss of epithelial cell polarity, the separation into individual cells and subsequent dispersion after the acquisition of cell motility (see, Vincent-Salomon et al., Breast Cancer Res. 2003; 5(2): 101-106). Classes of molecules that change in expression, distribution, and/or function during EMT, and that are causally involved, include growth factors (e.g., transforming growth factor-β (TGF-β), wnts), transcription factors (e.g., Snail, SMAD, LEF, and nuclear (3-catenin), molecules of the cell-to-cell adhesion axis (cadherins, catenins), cytoskeletal modulators (Rho family), and extracellular proteases (matrix metalloproteinases, plasminogen activators) (see, Thompson et al., Cancer Research 65, 5991-5995, Jul. 15, 2005). In specific embodiments, EMT refers to a process whereby epithelial cancer cells take on a mesenchymal phenotype, which may be associated with metastasis. These mesenchymal cells may display reduced adhesiveness, increased motility and invasiveness and are relatively resistant to immunotherapeutic agents, chemotherapeutic agents and/or radiation (e.g., treatments that target rapidly dividing cells).

The term “epitope” refers to that portion of a molecule capable of being recognized by and bound by an antibody at one or more of the antibody's antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “non-linear epitope” or “conformational epitope” comprises non-contiguous polypeptides (or amino acids) within the antigenic protein to which an antibody specific to the epitope binds. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to a target antigen (e.g., PD-1), e.g., the antibodies compete for binding to the antigen.

The term “exhaustion” and its grammatical equivalents refer to T-cell exhaustion as a state of T-cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that [symbol] ctor or memory T-cells. Exhaustion prevents optimal cont [symbol] tumors. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The terms “level of expression” or “expression level” in general are used interchangeably and generally refer to the amount of a biomarker in a sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (e.g., transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., T-cell dysfunctional disorder) or an internal control (e.g., housekeeping biomarker).

“Reduced expression”, “reduced expression levels”, or “reduced levels” refers to a decreased expression or decreased levels of a biomarker in an individual relative to a control, such as an individual or individuals who are not suffering from the disease or disorder (e.g., T-cell dysfunctional disorder) or an internal control (e.g., housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

A “growth inhibitory agent” when used herein refers to a compound or composition which inhibits growth of a cell either in vitro or in vivo. In one embodiment, growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents include agents that block cell cycle progression (at a place other than S phase), such as agents [symbol] rrest and M-phase arrest. Classical M-phase blockers incl [symbol] istine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “immune effector cells” in the context of the present invention relates to cells which exert effector functions during an immune reaction. For example, such cells secrete cytokines and/or chemokines, kill microbes, secrete antibodies, recognize infected or cancerous cells, and optionally eliminate such cells. For example, immune effector cells comprise T-cells (cytotoxic T-cells, helper T-cells, tumor infiltrating T-cells), B-cells, natural killer (NK) cells, lymphokine-activated killer (LAK) cells, neutrophils, macrophages, and dendritic cells.

The term “immune effector functions” in the context of the present invention includes any functions mediated by components of the immune system that result, for example, in the killing of virally infected cells or tumor cells, or in the inhibition of tumor growth and/or inhibition of tumor development, including inhibition of tumor dissemination and metastasis. Preferably, the immune effector functions in the context of the present invention are T-cell mediated effector functions. Such functions comprise in the case of a helper T-cell (CD4⁺ T-cell) the recognition of an antigen or an antigen peptide derived from an antigen in the context of MHC class II molecules by T-cell receptors, the release of cytokines and/or the activation of CD8+ lymphocytes (CTLs) and/or B-cells, and in the case of CTL the recognition of an antigen or an antigen peptide derived from an antigen in the context of MHC class I molecules by T-cell receptors, the elimination of cells presented in the context of MHC class I molecules, i.e., cells characterized by presentation of an antigen with class I MHC, for example, via apoptosis or perforin-mediated cell lysis, production of cytokines such as IFN-γ and TNF-α, and specific cytolytic killing of antigen expressing target cells.

The term “immune response” refers to any detectable response to a particular substance (such as an antigen or immunogen) by the immune system of a host mammal, such as innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The term “immunogenic” refers to the ability of a substance to cause, elicit, stimulate, or induce an immune response including an enhanced T-cell (e.g., CD8⁺ T-cell) immune response, or to improve, enhance, increase or prolong a pre-existing immune response, against a particular antigen, whether alone or when linked to a carrier, in the presence or absence of an adjuvant. [symbol] Immunogenicity” refers to the ability of a particular subst [symbol] immune response. Tumors are immunogenic and enhancing tumor immunogenicity aids in the clearance of the tumor cells by the immune response. Examples of enhancing tumor immunogenicity include treatment with a LSD inhibitor (e.g., a LSD1 inhibitor) and a PD-1 binding antagonist.

The term “infection” refers to invasion of body tissues by disease-causing microorganisms, their multiplication and the reaction of body tissues to these microorganisms and the toxins they produce. “Infection” includes but are not limited to infections by viruses, prions, bacteria, viroids, parasites, protozoans and fungi. Non-limiting examples of viruses include Retroviridae human immunodeficiency viruses, such as HIV-1 (also referred to as HTLV-III, LAV or HTLV-III/LAV, or HIV-III); and other isolates, such as HIV-LP); Picornaviridae (e.g., polio viruses, hepatitis A virus; enteroviruses, human Coxsackie viruses, rhinoviruses, echoviruses); Calciviridae (e.g., strains that cause gastroenteritis, including Norwalk and related viruses); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus, Metapneumovirus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, VACV, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); and astroviruses. Representative bacteria that are known to be pathogenic include pathogenic Pasteurella species (e.g., Pasteurella multocida), Staphylococcus species (e.g., Staphylococcus aureus), Streptococcus species (e.g., Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae), Neisseria species (e.g., Neisseria gonorrhoeae, Neisseria meningitidis), Escherichia species (e.g., enterotoxigenic E. coli (ETEC), enteropathogenic E. coli (EPEC), enterohemorrhagic E. coli (EHEC), and enteroinvasive E. coli (EIEC)), Bordetella species, Campylobacter species, Legionella species (e.g., Legionella pneumophila), Pseudomonas species, Shigella species, Vibrio species, Yersinia species, Salmonella species, Haemophilus species (e.g., Haemophilus influenzae), Brucella species, Francisella species, Bacteroides species, Clostridiium species (e.g., Clostridium difficile, Clostridium perfringens, Clostridium tetani), Mycobacteria species (e.g., M. tuberculosis, M. avium, M. intracellulare, M. kansaii, M. gordonae), Helicobacter pyloris, Borelia burgdorferi, Listeria monocytogenes, Chlamydia trachomatis, Enterococcus species, Bacillus anthracis, Corynebacterium diphtheriae, Erysipelothrix rhusiopathiae, Enterobacter aerogenes, Klebsiella pneumoniae, Fusobacterium nucleatum, Streptobacillus moniliformis, Treponema pallidium, Treponema pertenue, Leptospira, Rickettsia, and Actinomyces israeli. Non-limiting pathogenic fungi include Cryptococcus neoformans, Histoplasma capsulatum, Coccidioides immitis, Blastomyces dermatitidis, Candida albicans, Candida glabrata, Aspergillus fumigate, Aspergillus flavus, and Sporothrix schenckii. Illustrative [symbol] zoa, helminths, Plasmodium, such as Plasmodium falcipa [symbol] malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

As used herein, “instructional material” includes a publication, a recording, a diagram, or any other medium of expression which can be used to communicate the usefulness of the compositions and methods of the invention. The instructional material of the kit of the invention may, for example, be affixed to a container which contains the therapeutic or diagnostic agents of the invention or be shipped together with a container which contains the therapeutic or diagnostic agents of the invention.

The term “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

The term “leukocytes” or “white blood cell” as used herein refers to any immune cell, including monocytes, neutrophils, eosinophils, basophils, and lymphocytes.

As used herein, the term “LSD inhibitor” means an agent that decreases or inhibits the function or biological activity of a LSD polypeptide (e.g., LSD1—also known as lysine-specific histone demethylase 1A; lysine (K)-specific demethylase 1 (KDM1); lysine (K)-specific demethylase 1A (KDM1A); BRAF35-HDAC complex protein BHC110; FAD-binding protein BRAF35-HDAC complex, 110 kDa subunit; amine oxidase (flavin containing) domain 2 (AOF2); lysine-specific histone demethylase 1; RP1-18439.1—and LSD2—also known as lysine-specific histone demethylase 1B (KDM1B); amine oxidase flavin-containing 1 (AOF1); amine oxidase (flavin-containing) domain 1; flavin-containing amine oxidase domain-containing protein 1; lysine-specific histone demethylase 2; or the expression of a LSD gene (e.g., LSD1—also known as KDM1A; AOF2; BHC110; KDM1—and LSD2—also known as KDM1B; AOF1; bA20487.3; C6orf193; dJ298J15.2).

The term “lymphocytes” as used herein refers to cells of the immune system which are a type of white blood cell. Lymphocytes include, but are not limited to, T-cells (cytotoxic and helper T-cells), B-cells and natural killer cells (NK cells). The term “tumor infiltrating lymphocyte” as used herein refers to lymphocytes that are present in a solid tumor. The term “circulating lymphocyte” as used herein refers to lymphocytes that are present in the circulation (e.g., present in blood).

By “memory T effector cells” is meant a subset of T-cells including CTL and helper T-cells that have previously encountered and responded to their cognate antigen; thus, the term antigen-experienced T-cell is often applied. Such T-cells can recognize foreign microbes, such as bacteria or viruses, as well as cancer cells. Memory T effector cells have become “experienced” by having encountered antigen during a prior infection, encounter with cancer, or previous vaccination. At a second encounter with the microbe, memory T effector cells can reproduce to mount a faster and stronger immune response than the first time the immune system responded to the microbe. This behavior is utilized in T lymphocyte proliferation assays, which can reveal exposure to specific antigens. [symbol] he term “mesenchymal phenotype” is understood in the a [symbol] ified by morphological, molecular and/or functional characteristics. For example, mesenchymal cells generally have an elongated or spindle-shaped appearance, express the mesenchymal markers vimentin, fibronectin and N-cadherin, divide slowly or are non-dividing and/or have relatively high levels of motility, invasiveness and/or anchorage-independent growth as compared with epithelial cells.

As used herein, the term “mesenchymal-to-epithelial transition” (MET) is a reversible biological process that involves the transition from motile, multipolar or spindle-shaped mesenchymal cells to planar arrays of polarized cells called epithelia. MET is the reverse process of EMT. METs occur in normal development, cancer metastasis, and induced pluripotent stem cell reprogramming. In specific embodiments, MET refers to the reprogramming of cells that have undergone EMT to regain one or more epithelial characteristics (e.g., as described above). For example, such cells typically exhibit reduced motility and/or invasiveness and/or are rapidly dividing, and may thereby regain sensitivity to immunotherapeutics and/or cytotoxic agents.

The term “multiplex-PCR” refers to a single PCR reaction carried out on nucleic acid obtained from a single source (e.g., an individual) using more than one primer set for the purpose of amplifying two or more DNA sequences in a single reaction.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting an immune response, including an immune response with enhanced T-cell activation. However, it will be understood that the aforementioned terms do not imply that symptoms are present.

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

As used herein, the term “PD-1” refers to any form of PD-1 and variants thereof that retain at least part of the activity of PD-1. Unless indicated differently, such as by specific reference to human PD-1, PD-1 includes all mammalian species of native sequence PD-1, e.g., [symbol] eline, equine, and bovine. One exemplary human PD-1 is [symbol] Accession Number Q15116.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In some embodiments, a PD-1 binding antagonist reduces the negative co-stimulatory signal mediated by or through cell surface proteins expressed on T-cells mediated through PD-1 so as to render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is CT-011 (pidilizumab). In still another specific aspect, a PD-1 binding antagonist is AMP-224.

In the context of the present invention the term “priming” refers to the induction of a first contact of the T-cell (typically a naïve T-cell) with its specific antigen (e.g., by antigen-presenting cells presenting the antigen to T-cells), which causes the differentiation of the T-cell into an effector-T cell (e.g., a cytotoxic T cell or a T helper cell).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., feces), tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC). [symbol] “reference sample”, “reference cell”, “reference tissue”, [symbol] ntrol cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, Calif., USA) using standard defaults as used in the reference manual accompanying the software.

As used herein a “small molecule” refers to a compound that has a molecular weight of less than 3 kilodalton (kDa), and typically less than 1.5 kilodalton, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic [symbol] metal)) that has a molecular weight of less than 3 kilodal [symbol] kilodalton, or even less than about 1 kDa.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50.degree. C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

As used herein, the term “synergistic” means that the therapeutic effect of a LSD inhibitor when administered in combination with a PD-1 binding antagonist (or vice-versa) or when administered in combination with a PD-1 binding antagonist and a chemotherapeutic agent (“anti-PD-1-chemo combination”), is greater than the predicted additive therapeutic effects of the LSD inhibitor and the PD-1 binding antagonist, or the LSD inhibitor and the anti-PD-1-chemo combination, when administered alone. The term “synergistically effective amount” as applied to a LSD inhibitor and a PD-1 binding antagonist or anti-PD-1-chemo combination refers to the amount of each component in a composition (generally a pharmaceutical formulation), which is effective for enhancing immune effector function including any one or more of increased recognition of an antigen or an antigen peptide derived from an antigen in the context of MHC class II molecules by T-cell receptors, increased release of cytokines and/or the activation of CD8⁺ lymphocytes (CTLs) and/or B-cells, increased recognition of an antigen or an antigen peptide derived from an antigen in the context of MHC class I molecules by T-cell receptors, increased elimination of cells presented in the context of MHC class I molecules, i.e., cells characterized by presentation of an antigen with [symbol] example, via apoptosis or perforin-mediated cell lysis, inc [symbol] cytokines such as IFN-γ and TNF-α, and increased specific cytolytic killing of antigen expressing target cells, and which produces an effect which does not intersect, in a dose-response plot of the dose of LSD inhibitor versus a dose of PD-1 binding antagonist or anti-PD-1-chemo combination versus enhancing immune effector function as illustrated for example above, either the dose LSD inhibitor axis or PD-1 binding antagonist axis or the anti-PD-1-chemo combination axis. The dose response curve used to determine synergy in the art is described for example by Sande et al. (see, p. 1080-1105 in A. Goodman et al., ed., the Pharmacological Basis of Therapeutics, MacMillan Publishing Co., Inc., New York (1980)). The optimum synergistic amounts can be determined, using a 95% confidence limit, by varying factors such as dose level, schedule and response, and using a computer-generated model that generates isobolograms from the dose response curves for various combinations of the LSD inhibitor and the PD-1 binding antagonist or anti-PD-1-chemo combination. The highest enhancement of immune effector function on the dose response curve correlates with the optimum dosage levels.

A “T-cell dysfunctional disorder” is a disorder or condition of T-cells characterized by decreased responsiveness to antigenic stimulation. In a particular embodiment, a T-cell dysfunctional disorder is a disorder that is specifically associated with inappropriate increased signaling through PD-1. In another embodiment, a T-cell dysfunctional disorder is one in which T-cells are anergic or have decreased ability to secrete cytokines, proliferate, or execute cytolytic activity. In a specific aspect, the decreased responsiveness results in ineffective control of a pathogen or tumor expressing an immunogen. Examples of T-cell dysfunctional disorders characterized by T-cell dysfunction include unresolved acute infection, chronic infection and tumor immunity.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a T-cell dysfunctional disorder are mitigated or eliminated, including, but are not limited to, reducing the proliferation of (or destroying) cancerous cells, reducing pathogen infection, decreasing symptoms resulting from the disease, increasing the quality of life of those suffering from the disease, decreasing the dose of other medications required to treat the disease, and/or prolonging survival of individuals.

As used herein, the expressions “Treg” and “regulatory T-cells”, formerly known as suppressor T-cells, refer to T lymphocytes that maintain immunological tolerance. During an immune response, Tregs inhibit T cell-mediated immunity and suppress auto-reactive T cells that have escaped negative selection within the thymus. Adaptive Treg cells (called Th3 or Tr 1 cells) are thought to be generated during an immune response. Naturally occurring Treg cells (CD4⁺CD25⁺FoxP3⁺ Treg cells) are generated in the thymus and have been linked to interactions between developing T-cells with both myeloid (CD11c⁺) and plasmacytoid (CD123⁺) dendritic cells that have been activated with the cytokine thymic stromal lymphopoietin (TSLP). The presence of FoxP3 in naturally occurring Treg cells distinguishes them from other T-cells.

“Tumor,” as used herein, refers to all neoplastic cell growth and proliferation, whether malignant or benign, and all pre-cancerous and cancerous cells and tissues. The terms [symbol] ous”, “cell proliferative disorder”, “proliferative disorder” [symbol] disorder” and “tumor” are not mutually exclusive as referred to herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

As used herein, underscoring or italicizing the name of a gene shall indicate the gene, in contrast to its protein product, which is indicated by the name of the gene in the absence of any underscoring or italicizing. For example, “LSD1” shall mean the LSD1 gene, whereas “LSD1” shall indicate the protein product or products generated from transcription and translation and/or alternative splicing of the “LSD1” gene.

Each embodiment described herein is to be applied mutatis mutandis to each and every embodiment unless specifically stated otherwise.

### 2. Agents for Enhancing T Cell Function

The present invention is based in part of the determination that exposure of functionally repressed T-cells of a mesenchymal phenotype to LSD inhibitors, including LSD1 inhibitors, results in epigenetic reprogramming of the T-cells with de-repression of their immune effector function, including elevated expression of biomarkers of T-cell activation and effector capacity (e.g., IFN-γ, TNF-α, Ki67 and TBET), decreased expression of biomarkers of T-cell exhaustion (e.g., EOMES), as well as increased activation and proliferation of T-cells, including effector and memory T-cells. The present inventors have also found that LSD inhibitor-mediated epigenetic reprogramming confers enhanced susceptibility of exhausted T-cells to reinvigoration by PD-1 binding antagonists.

Thus, in accordance with the present invention, compositions and methods are provided that take advantage of a LSD inhibitor (e.g., an inhibitor of LSD demethylase activity or an inhibitor of LSD nuclear translocation/localization) and a PD-1 binding antagonist to enhance immune effector function, and/or to enhance T-cell (e.g., CD8⁺ T-cell or CD4⁺ T-cell) function, including increasing T-cell activation and enhancing susceptibility of exhausted T-cells to reinvigoration by PD-1 binding antagonists. The methods and compositions of the present invention are thus particularly useful in the treatment of T-cell dysfunctional disorders including cancers and infections.

2.1 LSD Inhibitors

The LSD inhibitor includes and encompasses any active agent that reduces the accumulation, function or stability of a LSD; or decrease expression of a LSD gene, and such inhibitors include without limitation, small molecules and macromolecules such as nucleic acids, peptides, polypeptides, peptidomimetics, carbohydrates, polysaccharides, lipopolysaccharides, lipids or other organic (carbon containing) or inorganic molecules. Preferred LSD inhibitors are ones that bind to LSD and inhibit its enzymatic activity and/or its nuclear localization. In specific embodiments, these LSD inhibitors are specific or selective LSD inhibitors.

In some embodiments, the LSD inhibitor is an antagonistic nucleic acid molecule that functions to inhibit the transcription or translation of LSD (e.g., LSD1 or LSD2) transcripts. Representative transcripts of this type include nucleotide sequences corresponding to any one the following sequences: (1) human LSD1 nucleotide sequences as set forth for example in GenBank [symbol] M_015013.3, NP_001009999.1, and NM_001009999.2; h [symbol] de sequences as set forth for example in GenBank Accession No. NM_153042.3; (2) nucleotide sequences that share at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) nucleotide sequences that hybridize under at least low, medium or high stringency conditions to the sequences referred to in (1); (4) nucleotide sequences that encode any one of the following amino acid sequences: human LSD1 amino acid sequences as set forth for example in GenPept Accession Nos. NP_055828.2, NP_001009999.1 and O60341.2; human LSD2 amino acid sequences as set forth for example in GenPept Accession Nos. NP_694587.3; (5) nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (4).

Illustrative antagonist nucleic acid molecules include antisense molecules, aptamers, ribozymes and triplex forming molecules, RNAi and external guide sequences. The nucleic acid molecules can act as effectors, inhibitors, modulators, and stimulators of a specific activity possessed by a target molecule, or the functional nucleic acid molecules can possess a de novo activity independent of any other molecules.

Antagonist nucleic acid molecules can interact with any macromolecule, such as DNA, RNA, polypeptides, or carbohydrate chains. Thus, antagonist nucleic acid molecules can interact with LSD (e.g., LSD1 or LSD2) mRNA or the genomic DNA of LSD (e.g., LSD1 or LSD2) or they can interact with a LSD polypeptide e.g., LSD1 or LSD2). Often antagonist nucleic acid molecules are designed to interact with other nucleic acids based on sequence homology between the target molecule and the antagonist nucleic acid molecule. In other situations, the specific recognition between the antagonist nucleic acid molecule and the target molecule is not based on sequence homology between the antagonist nucleic acid molecule and the target molecule, but rather is based on the formation of tertiary structure that allows specific recognition to take place.

In some embodiments, anti-sense RNA or DNA molecules are used to directly block the translation of LSD (e.g., LSD1 or LSD2) by binding to targeted mRNA and preventing protein translation. Antisense molecules are designed to interact with a target nucleic acid molecule through either canonical or non-canonical base pairing. The interaction of the antisense molecule and the target molecule may be designed to promote the destruction of the target molecule through, for example, RNAseH mediated RNA-DNA hybrid degradation. Alternatively the antisense molecule may be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Non-limiting methods include in vitro selection experiments and DNA modification studies using DMS and DEPC. In specific examples, the antisense molecules bind the target molecule with a dissociation constant (K_{d}) less than or equal to 10⁻⁶, 10⁻⁸, 10⁻¹°, or 10⁻¹². In specific embodiments, antisense oligodeoxyribonucleotides derived from the translation initiation site, e.g., between −10 and +10 regions are employed. [symbol] ptamers are molecules that interact with a target molecul [symbol] ific way. Aptamers are generally small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kds from the target molecule of less than 10⁻¹² M. Suitably, the aptamers bind the target molecule with a Kd less than 10⁻⁶, 10⁻⁸, 10⁻¹°, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is desirable that an aptamer have a K_{d} with the target molecule at least 10-, 100-, 1000-, 10,000-, or 100,000-fold lower than the K_{d} with a background-binding molecule. A suitable method for generating an aptamer to a target of interest (e.g., PHD, FIH-1 or vHL) is the “Systematic Evolution of Ligands by EXponential Enrichment” (SELEX™). The SELEX™ method is described in U.S. Pat. Nos. 5,475,096 and 5,270,163 (see also WO 91/19813). Briefly, a mixture of nucleic acids is contacted with the target molecule under conditions favorable for binding. The unbound nucleic acids are partitioned from the bound nucleic acids, and the nucleic acid-target complexes are dissociated. Then the dissociated nucleic acids are amplified to yield a ligand-enriched mixture of nucleic acids, which is subjected to repeated cycles of binding, partitioning, dissociating and amplifying as desired to yield highly specific high affinity nucleic acid ligands to the target molecule.

In other embodiments, anti-LSD (e.g., anti-LSD1 or LSD2) ribozymes are used for catalyzing the specific cleavage of LSD (e.g., LSD1 or LSD2) RNA. The mechanism of ribozyme action involves sequence specific hybridization of the ribozyme molecule to complementary target RNA, followed by a endonucleolytic cleavage. There are several different types of ribozymes that catalyze nuclease or nucleic acid polymerase type reactions, which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo. Representative ribozymes cleave RNA or DNA substrates. In some embodiments, ribozymes that cleave RNA substrates are employed. Specific ribozyme cleavage sites within potential RNA targets are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base pairing. Triplex molecules are preferred because they can bind target regions with high affinity and specificity. It is generally desirable that the triplex forming molecules bind the target molecule with a K_{d} less than 10⁻⁶, 10⁻⁸, 10⁻¹°, or 10⁻¹².

External guide sequences (EGSs) are molecules that bind a target nucleic acid molecule forming a complex, and this complex is recognized by RNAse P, which cleaves the target [symbol] an be designed to specifically target a RNA molecule of c [symbol] in processing transfer RNA (tRNA) within a cell. Bacterial RNAse P can be recruited to cleave virtually any RNA sequence by using an EGS that causes the target RNA: EGS complex to mimic the natural tRNA substrate. Similarly, eukaryotic EGS/RNAse P-directed cleavage of RNA can be utilized to cleave desired targets within eukaryotic cells.

In other embodiments, RNA molecules that mediate RNA interference (RNAi) of a LSD (e.g., LSD1 or LSD2) gene or LSD (e.g., LSD1 or LSD2) transcript can be used to reduce or abrogate gene expression. RNAi refers to interference with or destruction of the product of a target gene by introducing a single-stranded or usually a double-stranded RNA (dsRNA) that is homologous to the transcript of a target gene. RNAi methods, including double-stranded RNA interference (dsRNAi) or small interfering RNA (siRNA), have been extensively documented in a number of organisms, including mammalian cells and the nematode C. elegans (Fire et al., 1998. Nature 391, 806-811). In mammalian cells, RNAi can be triggered by 21- to 23-nucleotide (nt) duplexes of small interfering RNA (siRNA) (Chiu et al., 2002 Mol. Cell. 10:549-561; Elbashir et al., 2001. Nature 411:494-498), or by micro-RNAs (miRNA), functional small-hairpin RNA (shRNA), or other dsRNAs which are expressed in vivo using DNA templates with RNA polymerase III promoters (Zeng et al., 2002. Mol. Cell 9:1327-1333; Paddison et al., 2002. Genes Dev. 16:948-958; Lee et al., 2002. Nature Biotechnol. 20:500-505; Paul et al., 2002. Nature Biotechnol. 20:505-508; Tuschl, T., 2002. Nature Biotechnol. 20:440-448; Yu et al., 2002. Proc. Natl. Acad. Sci. USA 99(9):6047-6052; McManus et al., 2002. RNA 8:842-850; Sui et al., 2002. Proc. Natl. Acad. Sci. USA 99(6):5515-5520).

In specific embodiments, dsRNA per se and especially dsRNA-producing constructs corresponding to at least a portion of a LSD (e.g., LSD1 or LSD2) gene are used to reduce or abrogate its expression. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a LSD (e.g., LSD1 or LSD2) gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts, which then anneal to form a dsRNA having homology to a target gene.

Absolute homology is not required for RNAi, with a lower threshold being described at about 85% homology for a dsRNA of about 200 base pairs (Plasterk and Ketting, 2000, Current Opinion in Genetics and Dev. 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are suitably at least about 100 nucleotides in length. RNA-encoding constructs that express a single RNA designed to form a dsRNA via internal folding are usually at least about 200 nucleotides in length.

The promoter used to express the dsRNA-forming construct may be any type of promoter if the resulting dsRNA is specific for a gene product in the cell lineage targeted for [symbol] natively, the promoter may be lineage specific in that it [symbol] of a particular development lineage. This might be advantageous where some overlap in homology is observed with a gene that is expressed in a non-targeted cell lineage. The promoter may also be inducible by externally controlled factors, or by intracellular environmental factors.

In some embodiments, RNA molecules of about 21 to about 23 nucleotides, which direct cleavage of specific mRNA to which they correspond, as for example described by Tuschl et al. in U.S. 2002/0086356, can be utilized for mediating RNAi. Such 21- to 23-nt RNA molecules can comprise a 3′ hydroxyl group, can be single-stranded or double stranded (as two 21- to 23-nt RNAs) wherein the dsRNA molecules can be blunt ended or comprise overhanging ends (e.g., 5′, 3′).

In some embodiments, the antagonist nucleic acid molecule is a siRNA. siRNAs can be prepared by any suitable method. For example, reference may be made to International Publication WO 02/44321, which discloses siRNAs capable of sequence-specific degradation of target mRNAs when base-paired with 3′ overhanging ends, which is incorporated by reference herein. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER™ siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which is incorporated herein by reference.

Illustrative RNAi molecules (e.g., LSD (e.g., LSD1 or LSD2) siRNA and shRNA) are described in the art (e.g., Yang, et al., 2010. Proc. Natl. Acad. Sci. USA 107: 21499-21504 and He et al., 2012. Transcription 3:3: 1-16) or available commercially from Santa Cruz Biotechnology, Inc. (Santa Cruz, Calif., USA) and OriGene Technologies, Inc. (Rockville, Md., USA).

The present invention further contemplates peptide or polypeptide based inhibitor compounds. For example, BHC80 (also known as PHD finger protein 21A) forms part of a complex with LSD1 and can inhibit LSD1 demethylase activity. Accordingly, the present invention further contemplates the use of BHC80 or biologically active fragments thereof for inhibiting LSD1 enzymatic activity. Amino acid sequences of BHC80 polypeptides, and nucleotide sequences encoding BHC80 polypeptides, are publicly available. In this regard, reference may be made for example to GenBank Accession No. NP057705 for a Homo sapiens BHC80 amino acid sequence; and GenBank NM016621 for a nucleotide sequence encoding the amino acid sequence set forth in GenBank Accession No. NP057705; 2) GenBank Accession No. NP620094 for a Mus musculus [symbol] d sequence; and GenBank N M138755 for a nucleotide se [symbol] amino acid sequence set forth in GenBank Accession No. NP620094; 3) GenBank Accession No. NP00118576.1 for a Gallus gallus BHC80 amino acid sequence; and GenBank NM001199647 for a nucleotide sequence encoding the amino acid sequence set forth in GenBank Accession No. NP00118576.1; and 4) GenBank Accession No. DAA21793 for a Bos taurus BHC80 amino acid sequence.

Illustrative BHC80 polypeptides are selected from the group consisting of: (1) a polypeptide comprising an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with the amino acid sequence listed in any one of the GenBank BHC80 polypeptide entries noted above; (2) a polypeptide comprising an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with the amino acid sequence listed in any one of the GenBank BHC80 polypeptide entries noted above; (3) a polypeptide comprising an amino acid sequence that is encoded by a nucleotide sequence that hybridizes under at least low, medium or high stringency conditions to the nucleotide sequence listed in any one of the GenBank BHC80 polynucleotide entries noted above; (4) a polypeptide comprising an amino acid sequence that is encoded by a nucleotide sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity to the nucleotide sequence listed in any one of the GenBank BHC80 polynucleotide entries noted above; and (5) a fragment of a polypeptide according to any one of (1) to (4), which inhibits LSD1 enzymatic activity.

A BHC80 polypeptide can be introduced into a cell by delivering a polypeptide per se, or by introducing into the cell a BHC80 nucleic acid comprising a nucleotide sequence encoding a BHC80 polypeptide. In some embodiments, a BHC80 nucleic acid comprises a nucleotide sequence selected from: (1) a BHC80 nucleotide sequence listed in any one of the GenBank BHC80 polynucleotide entries noted above; (2) a nucleotide sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) a nucleotide sequence that hybridizes under at least low, medium or high stringency conditions to the sequences referred to in (1); (4) a nucleotide sequence that encodes an amino acid sequence listed in any one of the GenBank BHC80 polypeptide entries noted above; (5) a nucleotide sequence that encodes an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and a nucleotide sequence that encodes an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (4).

The BHC80 nucleic acid can be in the form of a recombinant expression vector. The BHC80 nucleotide sequence can be operably linked to a transcriptional control element(s), e.g., a promoter, in the expression vector. Suitable vectors include, e.g., recombinant retroviruses, lentiviruses, and adenoviruses; retroviral expression vectors, lentiviral expression vectors, nucleic acid expression vectors, and plasmid expression vectors. In some cases, the expression vector is integrated into the genome of a cell. In other cases, the expression vector persists in an episomal state in a cell. [symbol] uitable expression vectors include, but are not limited to, [symbol] iral vectors based on vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641 648, 1999; Ali et al., Hum Mol Genet. 5:591 594, 1996; Srivastava in WO 93/09239, Samulski et al., 3. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

The present invention also contemplates small molecule agents that reduce enzymatic activity of LSDs (e.g., LSD1 or LSD2).

Small molecule agents that reduce enzymatic activity of LSD1 that are suitable for use in the present invention include monoamine oxidase (MAO) inhibitors that also inhibit LSD1 enzymatic activity; polyamine compounds that inhibit LSD1 enzymatic activity; phenylcyclopropylamine derivatives that inhibit LSD1 enzymatic activity; and the like.

Non-limiting examples of MAO inhibitors include MAO-A-selective inhibitors, MAO-B-selective inhibitors, and MAO non-selective inhibitors. Illustrative examples of MAO inhibitors include reported inhibitors of the MAO-A isoform, which preferentially deaminates 5-hydroxytryptamine (serotonin) (5-HT) and norepinephrine (NE), and/or the MAO-B isoform, which preferentially deaminates phenylethylamine (PEA) and benzylamine (both MAO-A and MAO-B metabolize Dopamine (DA)). In various embodiments, MAO inhibitors may be irreversible or reversible (e.g., reversible inhibitors of MAO-A (RIMA)), and may have varying potencies against MAO-A and/or MAO-B (e.g., non-selective dual inhibitors or isoform-selective inhibitors).

In some embodiments, the MAO inhibitors are selected from: clorgyline; L-deprenyl; isocarboxazid (Marplan™); ayahuasca; nialamide; iproniazide; iproclozide; moclobemide (Aurorix™; 4-chloro-N-(2-morpholin-4-ylethyl)benzamide); phenelzine (Nardil™; (±)-2-phenylethylhydrazine); tranylcypromine (Parnate™; (±)-trans-2-phenylcyclopropan-1-amine) (the congeneric of phenelzine); toloxatone; levo-deprenyl (Selegiline™); harmala; RIMAs (e.g., moclobemide, described in Da Prada et al. (1989. J Pharmacol Exp Ther 248:400-414); brofaromine; and befloxatone, described in Curet et al. (1998. J Affect Disord 51: 287-30), lazabemide (Ro 19 6327), described in Ann. Neurol., 40(1): 99-107 (1996), and SL25.1131, described in Aubin et al. (2004. J. Pharmacol. Exp. Ther. 310: 1171-1182); selegiline hydrochloride (1-deprenyl, ELDEPRYL, ZELAPAR); dimethylselegilene; safinamide; rasagiline (AZILECT); bifemelane; desoxypeganine; harmine (also known as telepathine or banasterine); linezolid (ZYVOX, ZYVOXID); pargyline (EUDATIN, SUPIRDYL); dienolide kavapyrone desmethoxyyangonin; 5-(4-Arylmethoxyphenyl)-2-(2-cyanoethyl)tetrazoles; and the like.

Small molecule LSD1 inhibitors may also be selected from polyamine compounds as described for example by Woster et al. in U.S. Publication No. 2007/0208082, which is incorporated [symbol] ce in its entirety. Illustrative polyamine inhibitors of LSD [symbol] according to formula (I):

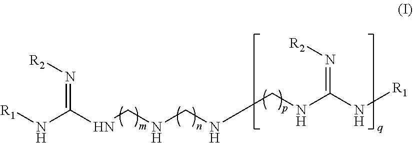

or a salt, solvate, or hydrate thereof, where n is an integer from 1 to 12; m and p are independently an integer from 1 to 5; q is 0 or 1; each R₁ is independently selected from the group consisting of CFCs alkyl, C₄-C₁₅ cycloalkyl, C₃-C₁₅ branched alkyl, C₆-C₂₀ aryl, C₆-C₂₀ heteroaryl, C₇-C₂₄ aralkyl, C₇-C₂₄ heteroaralkyl, and

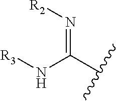

where R₃ is selected from the group consisting of C₁-C₅ alkyl, C₄-C₁₅ cycloalkyl, C₃-C₁₅ branched alkyl, C₆-C₂₀ aryl, C₆-C₂₀ heteroaryl, C₇-C₂₄ aralkyl and C₇-C₂₄ heteroaralkyl; and

each R₂ is independently selected from hydrogen or a C₁-C₅ alkyl.

A suitable polyamine compound is a compound of formula (I), wherein one or both R₁ is a C₆-C₂₀ aryl, such as a single ring aryl, including without limitation, a phenyl. In one embodiment, the compound is of the formula (I) and each R₁ is phenyl. In one embodiment, q is l, m and p are 3, and n is 4. In another embodiment, q is l, m and p are 3, and n is 7.

A suitable polyamine compound is a compound of formula (I), where at least one or both R₁ is a C₃-C₁₂ or a C₁-C₈ alkyl, such as a linear alkyl. One or both R₁ may be a C₁-C₈ linear alkyl, such as methyl or ethyl. In one embodiment, each R₁ is methyl. One or both R₁ may comprise or be a C₄-C₁₅ cycloalkyl group, such as a cycloalkyl group containing a linear alkyl group, where the cycloalkyl group is connected to the molecule either via its alkyl or cycloalkyl moiety. For instance, one or both R₁ may be cyclopropylmethyl or cyclohexylmethyl. In one embodiment, one R₁ is cyclopropylmethyl or cyclohexylmethyl and the other R₁ is a linear alkyl group, such as a linear C₁-C₅ unsubstituted alkyl group, including without limitation an ethyl group. In one embodiment, R₁ is a C₃-C₁₅ branched alkyl group such as isopropyl. When R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)_{z}CH₃ where y and z are independently an integer from 1 to 8. In one embodiment, R₁ is —(CH₂)₃NH₂.

In one embodiment, the compound is of the formula (I) where one or both R₁ is a C₇-C₂₄ substituted or unsubstituted aralkyl, which in one embodiment is an aralkyl connected to the molecule via its alkyl moiety (e.g., benzyl). In one embodiment, both R₁ are aralkyl moieties wherein the alkyl portion of the moiety is substituted with two aryl groups and the moiety is connected to the molecule via its alkyl group. For instance, in one embodiment one or both R₁ is a C₇-C₂₄ aralkyl wherein the alkyl portion is substituted with two phenyl groups, such as when R₁ is [symbol] or 2,2-dibenzylethyl. In one embodiment, both R₁ of for [symbol] diphenylethyl and n is 1, 2 or 5. In one embodiment, each R₁ of formula (I) is 2,2-diphenylethyl, n is 1, 2 or 5 and m and p are each 1.

In one embodiment, at least one R₁ is hydrogen. When one R₁ is hydrogen, the other R₁ may be any moiety listed above for R₁, including an aryl group such as benzyl. Any of the compounds of formula (I) listed above include compounds where at least one or both of R₂ is hydrogen or a C₁-C₈ substituted or unsubstituted alkyl. In one embodiment, each R₂ is an unsubstituted alkyl such as methyl. In another embodiment, each R₂ is hydrogen. Any of the compounds of formula (I) listed above may be compounds where q is 1 and m and p are the same. Accordingly, the polyaminoguanidines of formula (I) may be symmetric with reference to the polyaminoguanidine core (e.g., excluding R₁). Alternatively, the compounds of formula (I) may be asymmetric, e.g., when q is 0. In one embodiment, m and p are 1. In one embodiment, q is 0. In one embodiment, n is an integer from 1 to 5.

In some embodiments, the compound is a polyaminobiguanide or N-alkylated polyaminobiguanide. An N-alkylated polyaminobiguanide intends a polyaminobiguanide where at least one imine nitrogen of at least one biguanide is alkylated. In one embodiment, the compound is a polyaminobiguanide of the formula (I), or a salt, solvate, or hydrate thereof, where q is 1, and at least one or each R₁ is of the structure:

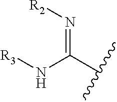

where each R₃ is independently selected from the group consisting of C₁-C₈ alkyl, C₆-C₂₀ aryl, C₅-C₂₀ heteroaryl, C₇-C₂₄ aralkyl, and C₇-C₂₄ heteroaralkyl; and each R₂ is independently hydrogen or a C₁-C₈ alkyl.

In one embodiment, in the polyaminobiguanide compound, at least one or each R₃ is a C₁-C₈ alkyl. For instance, when R₃ is a C₁-C₈ alkyl, the alkyl may be substituted with any substituent, including a primary, secondary, tertiary or quaternary amine. Accordingly, in one embodiment, R₃ is a C₁-C₈ alkyl group substituted with an amine such that R₃ may be e.g., alkyl-NH₂ or an alkyl-amine-alkyl moiety such as —(CH₂)₅NH(CH₂)₁CH₃ where y and z are independently an integer from 1 to 8. In one embodiment, R₃ is —(CH₂)₃NH₂. R₃ may also be a C₄-C₁₅ cycloalkyl or a C₃-C₁₅ branched alkyl. In one embodiment, at least one or each R₃ is a C₆-C₂₀ aryl. In one embodiment, q is l, m and p are 3, and n is 4. In another embodiment, q is l, m and p are 3, and n is 7.

In one embodiment, the compound is a polyaminobiguanide of formula (I) where at least one R₃ is a C₇-C₂₄ aralkyl, which in one embodiment is an aralkyl connected to the molecule via its alkyl moiety. In one embodiment, each R₃ is an aralkyl moiety where the alkyl portion of the moiety is substituted with one or two aryl groups and the moiety is connected to the molecule via its alkyl moiety. For instance, in one embodiment at least one or each R₃ is an aralkyl where the alkyl portion is substituted with two phenyl or benzyl groups, such as when R₃ is 2,2-diphenylethyl or 2,2-dibenzylethyl. In one embodiment, each R₃ is 2,2-diphenylethyl and n is 1, 2 or 5. In one embodiment, each R₃ is 2,2-diphenylethyl and n is 1, 2 or 5 and m and p are each 1. [symbol] ny of the polyaminobiguanide compounds of formula (I) [symbol] compounds where at least one or both of R₂ is hydrogen or a C₁-C₈ alkyl. In one embodiment, each R₂ is an unsubstituted alkyl, such as methyl. In another embodiment, each R₂ is a hydrogen.

Any of the polyaminobiguanide compounds of formula (I) listed above include compounds where q is 1 and m and p are the same. Accordingly, the polyaminobiguanides of formula (I) may be symmetric with reference to the polyaminobiguanide core. Alternatively, the compounds of formula (I) may be asymmetric. In one embodiment, m and p are 1. In one embodiment, q is 0. In one embodiment, n is an integer from 1 to 5. In one embodiment, q, m and p are each 1 and n is 1, 2 or 5.

It is understood and clearly conveyed by this disclosure that each R₁, R₂, R₃, m, n, p and q disclosed in reference to formula (I) intends and includes all combinations thereof the same as if each and every combination of R₁, R₂, R₃, m, n, p and q were specifically and individually listed.

Representative compounds of the formula (I) include, e.g.:

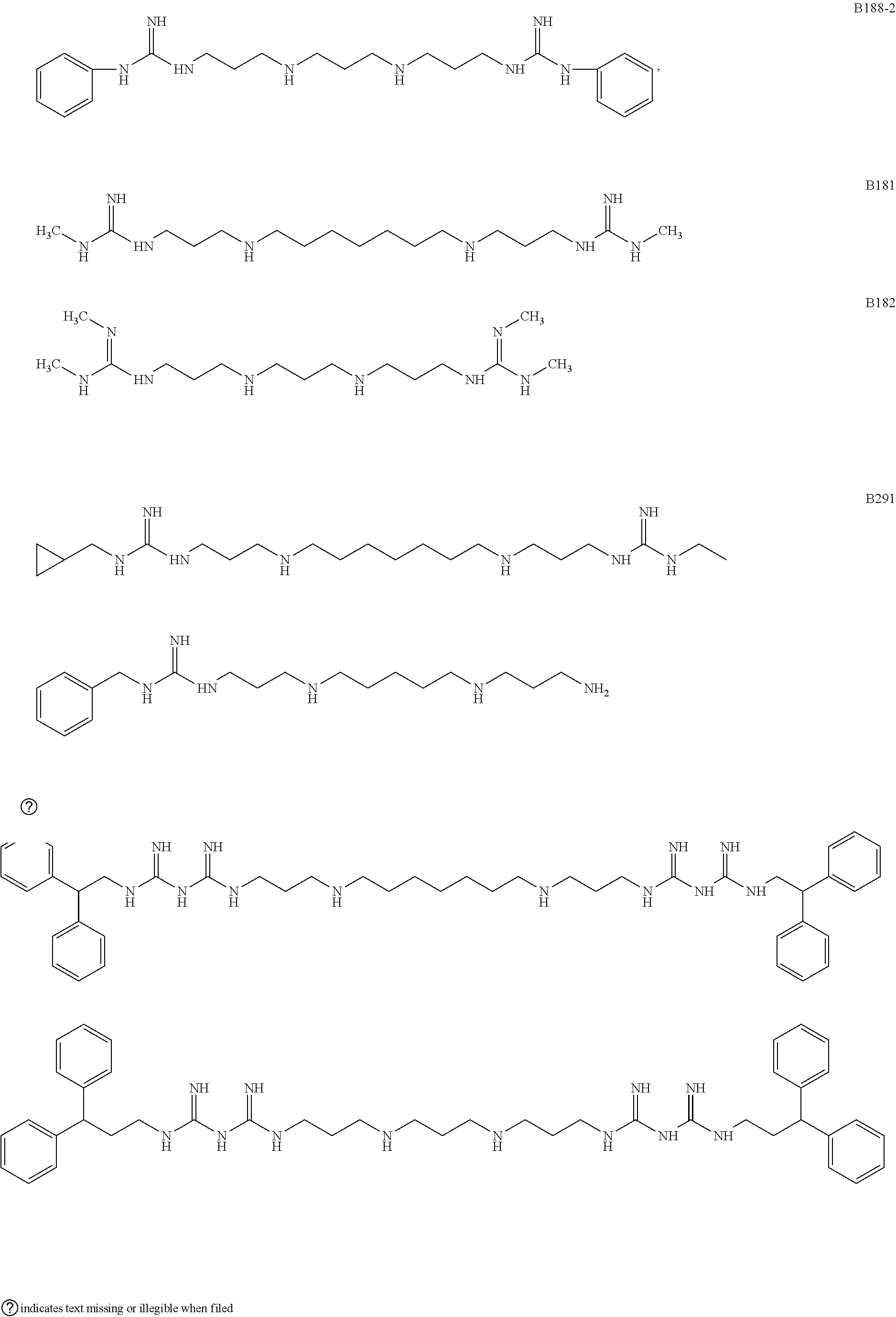

In certain embodiments, the polyamine compound is represented by the structure according to formula (II):

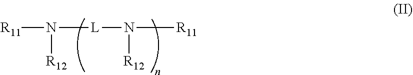

or a salt, solvate or hydrate thereof,

where n is 1, 2 or 3;

each L is independently a linker of from about 2 to 14 carbons in length, for example of about 2, 3, 4, 5, 6, 8, 10, 12 or 14 carbon atoms in length, where the linker backbone atoms may be saturated or unsaturated, usually not more than one, two, three, or four unsaturated atoms will be present in a tether backbone, where each of the backbone atoms may be substituted or unsubstituted (for example with a C₁-C₈ alkyl), where the linker backbone may include a cyclic group (for example, a cyclohex-1,3-diyl group where 3 atoms of the cycle are included in the backbone);

each R₁₂ is independently selected from hydrogen and a C₁-C₈ alkyl; and

each R₁₁ is independently selected from hydrogen, C₂-C₈ alkenyl, C₁-C₈ alkyl or C₃-C₈ branched alkyl (e.g., methyl, ethyl, tert-butyl, isopropyl, pentyl, cyclobutyl, cyclopropylmethyl, 3-methylbutyl, 2-ethylbutyl, 5-NH₂-pent-1-yl, propyl-1-ylmethyl(phenyl)phosphinate, dimethylbicyclo[3.1.1]heptyl)ethyl, 2-(decahydronaphthyl)ethyl and the like), C₅-C₂₀ aryl or heteroaryl, C₁-C₂₄ aralkyl or heteroaralkyl (2-phenylbenzyl, 4-phenylbenzyl, 2-benzylbenzyl, 3-benzylbenzyl, 3,3-diphenylpropyl, 3-(benzoimidazolyl)-propyl, 4-isopropylbenzyl, 4-fluorobenzyl, 4-tert-butylbenzyl, 3-imidazolyl-propyl, 2-phenylethyl and the like), —C(═O)—C₁-C₈ alkyl, —C(═O)—C₁-C₈ alkenyl, —C(═O)—C₁-C₃alkynyl, an amino-substituted cycloalkyl (e.g., a cycloalkyl group substituted with a primary, secondary, tertiary or quaternary amine, such as 5-NH₂-cycloheptyl, 3-NH₂-cyclopentyl and the like) and a C₂-C₈ alkanoyl (e.g., an alkanoyl substituted with a methyl and an alkylazide group).

In certain embodiments, each L is independently selected from: —CHR₁₃—(CH₂)ₘ—, —CHR₁₃—(CH₂)ₙ—CHR₁₃—, —(CH₂)ₘCHR₁₃—, —CH₂-A-CH₂— and —(CH₂)ₚ—

where:

m is an integer from 1 to 5;

A is (CH₂)ₘ, ethane-1,1-diyl or cyclohex-1,3-diyl; [symbol] is an integer from 2 to 14, such as 1, 2, 3, 4 or 5;

n is an integer from 1 to 12; and

R₁₃ is a C₁-C₈ alkyl.

A substituted aralkyl or heteroaralkyl with reference to formula (II) intends and includes alkanoyl moieties substituted with an aryl or heteroaryl group, i.e., —C(═O)-aryl, —C(═O)-aralkyl, —C(═O)-heteroaryl, and —C(═O)-heteroaralkyl. In one embodiment, the alkyl portion of the aralkyl or heteroaralkyl moiety is connected to the molecule via its alkyl moiety. For instance at least one or both of R₁₁ may be an aralkyl moiety such as 2-phenylbenzyl, 4-phenylbenzyl, 3,3,-diphenylpropyl, 2-(2-phenylethyl)benzyl, 2-methyl-3-phenylbenzyl, 2-napthylethyl, 4-(pyrenyl)butyl, 2-(3-methylnapthyl)ethyl, 2-(1,2-dihydroacenaphth-4-yl)ethyl and the like. In another embodiment, at least one or both of R₁₁ may be a heteroaralkyl moiety such as 3-(benzoimidazolyl)propanoyl, 1-(benzoimidazolyl)methanoyl, 2-(benzoimidazolyl)ethanoyl, 2-(benzoimidazolyl)ethyl and the like.

In certain embodiments, the compound of formula (II) comprises at least one moiety selected From the group consisting of t-butyl, isopropyl, 2-ethylbutyl, 1-methylpropyl, 1-methylbutyl, 3-butenyl, isopent-2-enyl, 2-methylpropan-3-olyl, ethylthiyl, phenylthiyl, propynoyl, 1-methyl-1H-pyrrole-2-yl; trifluoromethyl, cyclopropanecarbaldehyde, halo-substituted phenyl, nitro-substituted phenyl, alkyl-substituted phenyl, 2,4,6-trimethylbenzyl, halo-5-substituted phenyl (such as para-(F₃S)-phenyl, azido and 2-methylbutyl.

In certain embodiments, in formula (II), each R₁₁ is independently selected from hydrogen, n-butyl, ethyl, cyclohexylmethyl, cyclopentylmethyl, cyclopropylmethyl, cycloheptylmethyl, cyclohexyleth-2-yl, and benzyl.

In certain embodiments, the polyamine compound is of the structure of formula (II), where n is 3, such that the compound has a structure according to formula (III):

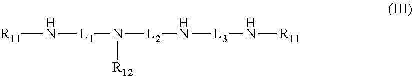

where L₁, L₂ and L₃ are independently selected from —CHR₁₃—(CH₂)ₘ—, —CHR₁₃—(CH₂)ₙ—CHR₁₃—, —(CH₂)ₘ—CHR₁₃—, —CH₂-A-CH₂— and —(CH₂)ₚ—

where m, A, p, n and R₁₃ are as defined above.

In certain embodiments, the polyamine compound is of the structure of formula (III) where: L₁ is —CHR₁₃—(CH₂)ₘ—; L₂ is —CHR₁₃—(CH₂)ₙ—CHR₁₃—; and L₃ is —(CH₂)ₘ—CHR₁₃—; where

R₁₁, R₁₂, R₁₃, m and n are as defined above.

In certain embodiments, the polyamine compound is of the structure of formula (III) where: L₁, L₂ and L₃ are independently —CH₂-A-CH₂—; and R₁₂ is hydrogen; where R₁₁ and A are as defined above. In particular embodiments, at least one of an A and an R₁₁ comprises an alkenyl moiety.

In certain embodiments, the polyamine compound is of the structure of formula (III) where: L₁, L₂ and L₃ are independently —(CH₂)ₚ— where p is as defined above; and Ru is hydrogen. In particular embodiments, for L₁ and L₃, p is an integer from 3 to 7, and for L₃ p is an integer from 3 to 14. [symbol] certain embodiments, the polyamine compound is of th [symbol] a (III) where: L₁, and L₃ are independently —(CH₂)ₚ—, L₂ is —CH₂-A-CH₂—; and R₁₂ is hydrogen; where R₁₂, p and A are as defined above. In particular embodiments, for L₁ and L₃, p is an integer from 2 to 6, and for L₃ A is (CH₂)^{x} where x is an integer from 1 to 5, or cyclohex-1,3-diyl.

In certain embodiments, the polyamine compound is of the structure of formula (II), where n is 2, such that the compound has a structure according to formula (IV):

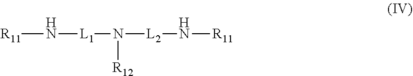

where L₁ and L₂ are independently selected from —CHR₁₃—(CH₂)ₘ—CHR₁₃—(CH₂)ₙ—CHR₁₃—, —(CH₂)ₙ, CHR₁₃—, —CH₂-A-CH₂— and —(CH₂)ₚ—

where m, A, p, n, and R₁₃ are as defined above.

In certain embodiments, the polyamine compound is of the structure of formula (IV) where: L₁ is —(CH₂)ₚ—; and L₂ is —(CH₂)ₘ—CHR₁₃—; where R₁₃, m and p are as defined above. In particular embodiments, for L₁ p is an integer from 3 to 10, and for L₂ n is an integer from 2 to 9.

In certain embodiments, the polyamine compound is of the structure of formula (IV) where: L₁ and L₂ are —(CH₂)ₚ—; where p is as defined above. In particular embodiments, p is an integer from 3 to 7.

In certain embodiments, the polyamine compound is of the structure of formula (II), where n is 1, such that the compound has a structure according to formula (V):

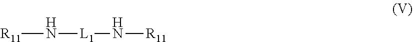

where L₁ is —(CH₂)ₚ— where p is as defined above. In particular embodiments, p is an integer from 2 to 6.

In particular embodiments, in formula (V), one R₁₁ is an amino-substituted cycloalkyl (e.g., a cycloalkyl group substituted with a primary, secondary, tertiary or quaternary amine) or a C₂-C₈ alkanoyl (which alkanoyl may be substituted with one or more substituents such as a methyl or an alkylazide group); and the other R₁₁ is a C₁-C₈ alkyl or a C₇-C₂₄ aralkyl.

Representative compounds of the formula (II) include, e.g.:

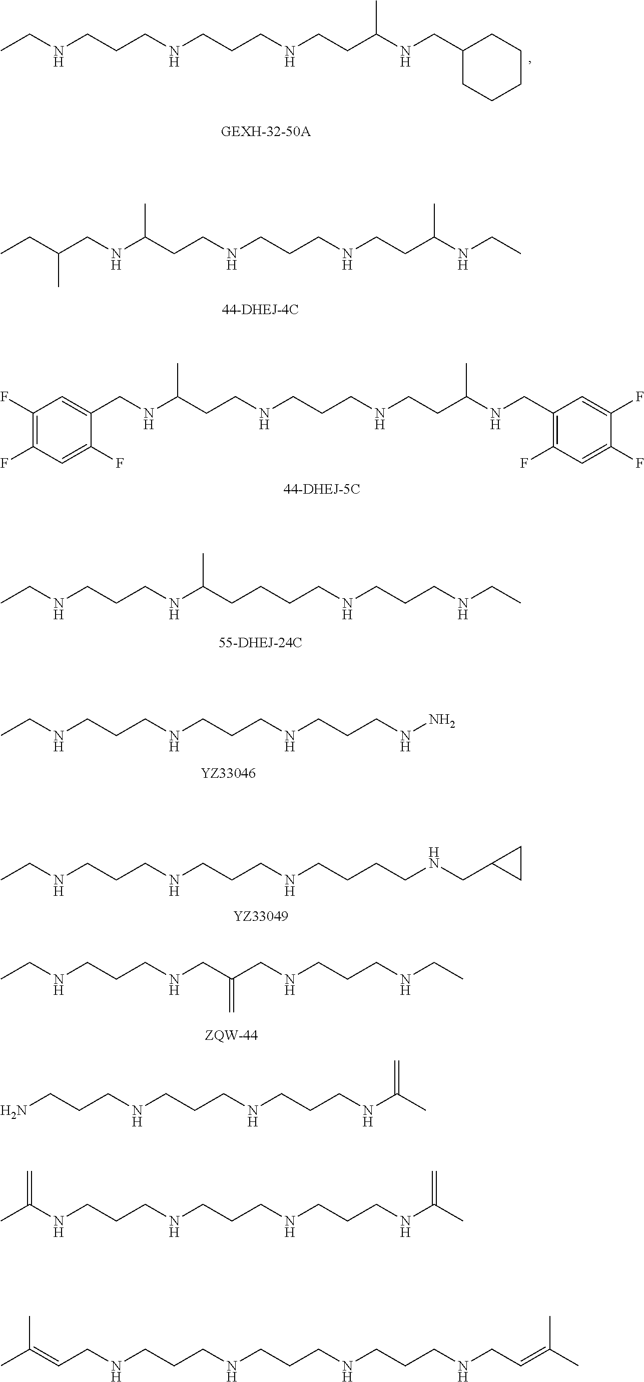

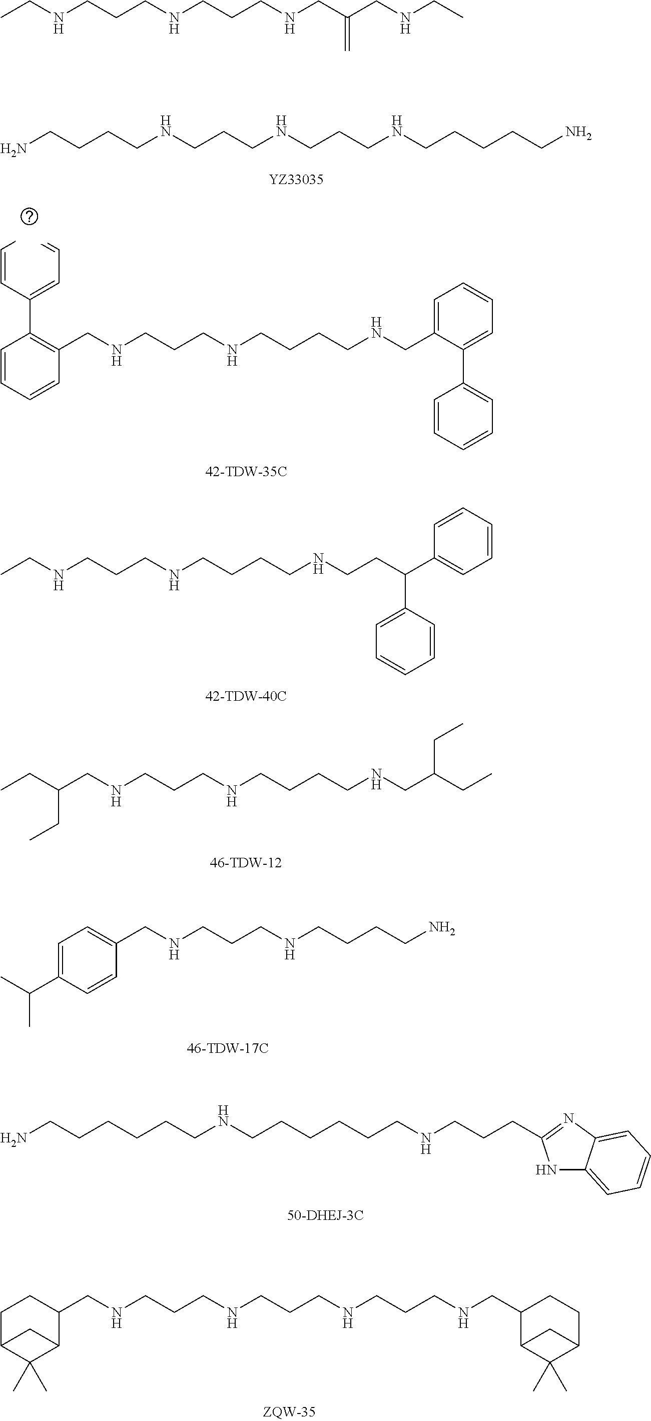

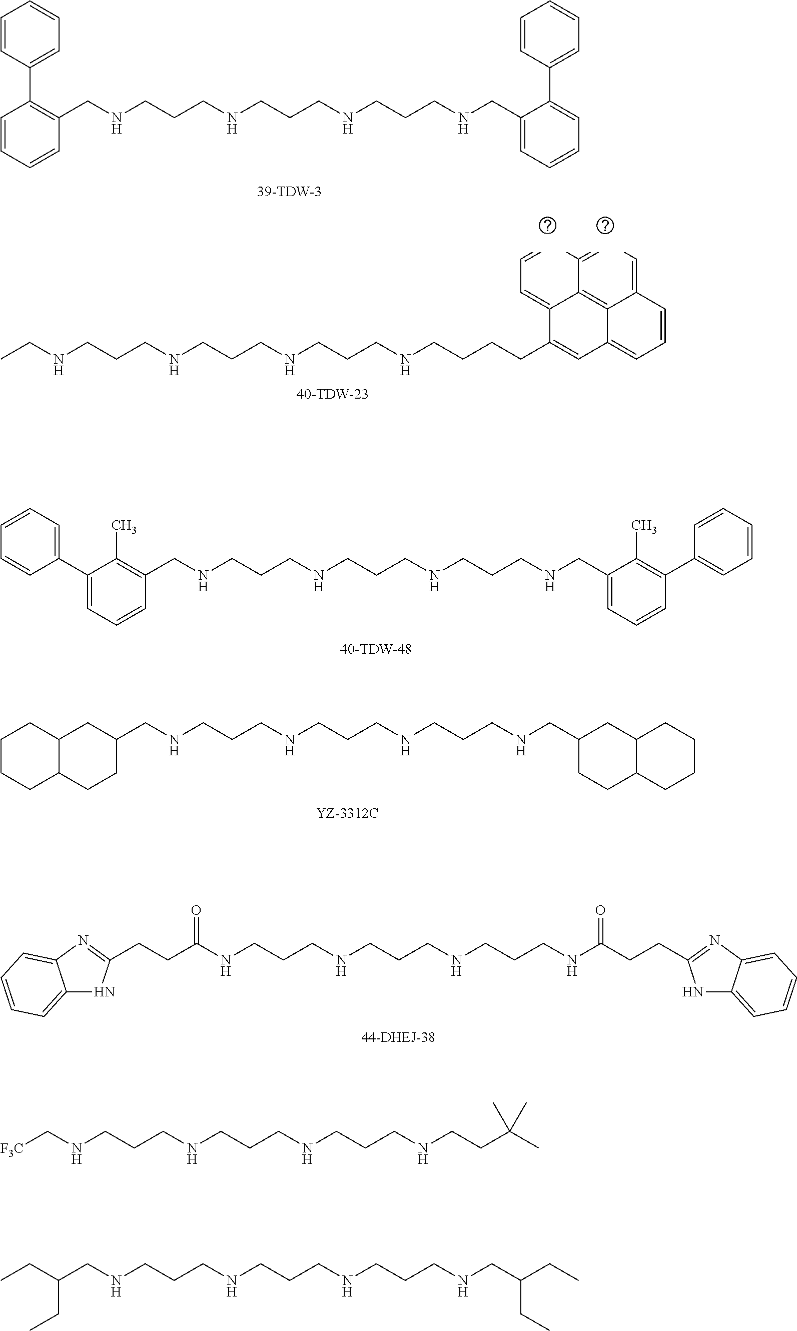

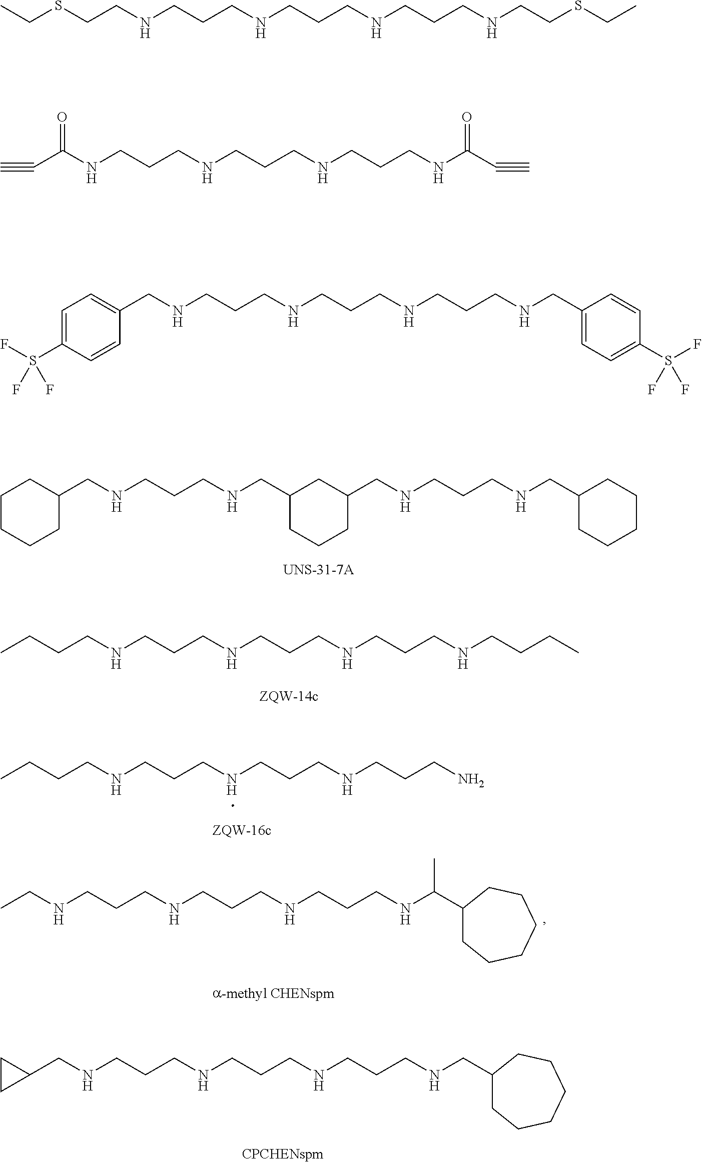

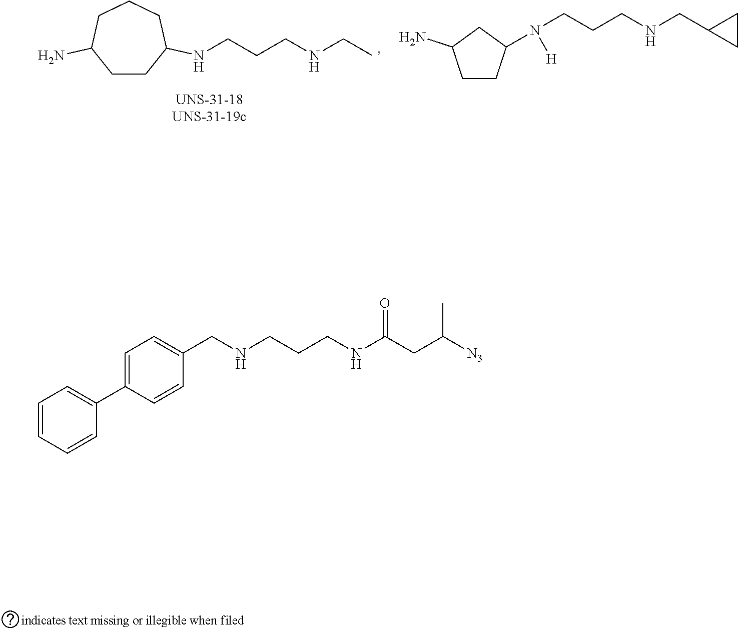 [SMILES: CCNCCCNC1CCCC(N)CC1 ; NC1CCC(NCCCNCC2CC2)C1 ; CC(CC(=O)NCCCNCc1ccc(-c2ccccc2)cc1)N=[N+]=[N-]]

Phenylcyclopropylamine derivatives that are inhibitors of include compounds represented by formula (VI):

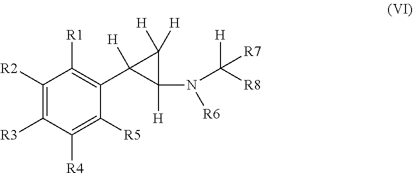

wherein:

each of R1-R5 is independently selected from H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R6 is H or alkyl; [symbol] 7 is H, alkyl, or cycloalkyl;

R8 is an -L-heterocyclyl wherein the ring or ring system of the -L-heterocyclyl has from 0 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido; or

R8 is -L-aryl wherein the ring or ring system of the -L-aryl has from 1 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

where each L is independently selected from —(CH₂)ₙ—(CH₂)ₙ—, —(CH₂)ₙNH(CH₂)ₙ—, —(CH₂)ₙO(CH₂)ₙ—, and —(CH₂)ₙS(CH₂)ₙ—, and where each n is independently chosen from 0, 1, 2, and 3;

or a pharmaceutically acceptable salt thereof.

In some cases, L is a covalent bond. In some cases, R6 and R7 are hydro. In some cases, one of R1-R5 is selected from -L-aryl, -L-heterocyclyl, and -L-carbocyclyl.

In some embodiments of the compound of formula (VI), the substituent or substituents on the R8 ring or ring system is/are selected from hydroxyl, halo, alkyl, alkoxy, cycloalkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃ alkyl), —S(═O)₂NH₂, —S(O)₂NH₂, —S(O)₂N(C₁₋₃ alkyl)₂, —S(═O)₂NH(C₁₋₃ alkyl), —CN, —NH₂, and —NO₂.

In certain embodiments, a compound of the invention is of formula (VI) where:

each R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocyclyl, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R6 is chosen from —H and alkyl;

R7 is chosen from —H, alkyl, and cycloalkyl;

R8 is chosen from —C(═O)NRxRy and —C(═O)Rz;

Rx when present is chosen from —H, alkyl, alkynyl, alkenyl, -L-carbocyclyl, -L-aryl, and -L-heterocyclyl, all of which are optionally substituted (except —H); [symbol] y when present is chosen from —H, alkyl, alkynyl, alkenyl, [symbol] ryl, and -L-heterocyclyl, all of which are optionally substituted (except —H), where Rx and Ry may be cyclically linked;

Rz when present is chosen from —H, alkoxy, -L-carbocyclyl, -L-heterocyclyl, -L-aryl, wherein the aryl, heterocyclyl, or carbocyclyl are optionally substituted; each L is a linker that links the main scaffold of formula (I) to a carbocyclyl, heterocyclyl, or aryl group, wherein the hydrocarbon portion of the linker -L- is saturated, partially saturated, or unsaturated, and is independently chosen from a saturated parent group having a formula of —(CH₂)ₙ—(CH₂)ₙ—, —(CH₂)ₙC(═O)(CH₂)—, —(CH₂)ₙC(═O)NH(CH₂)ₙ—, —(CH₂)ₙNHC(O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙ—O—(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8. According to this embodiment, optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido. In a more specific aspect of this embodiment, the optional substituents are 1 or 2 optional substituents chosen from halo, alkyl, aryl, and arylalkyl.

In certain embodiments, in formula (VI), R8 is —CORz, such that the compound is of the following structure:

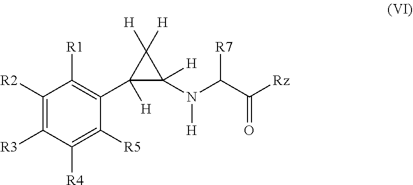

where: R1-R7 are described above; and Rz is -L-heterocyclyl which is optionally substituted with from 1-4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido, and wherein said -L- is independently chosen from —(CH₂)ₙ—(CH₂)ₙ—, —(CH₂)ₙNH(CH₂)ₙ—, (CH₂)ₙ—O—(CH₂)ₙ—, and —(CH₂)ₙS(CH₂)ₙ—, where each n is independently chosen from 0, 1, 2, and 3.

In a specific aspect of this embodiment, each L is independently chosen from —(CH₂)ₙ—(CH₂)ₙ— and —(CH₂)ₙ—O—(CH₂)ₙ—, where each n is independently chosen from 0, 1, 2, and 3. In a more specific aspect of this embodiment, each L is chosen from a bond, —CH₂—, —CH₂CH₂—, —OCH₂CH₂—, —CH₂OCH₂—, —CH₂CH₂CH₂—, —OCH₂CH₂CH₂—, and —CH₂OCH₂CH₂—. In an even more specific aspect, each L is chosen from a bond, —CH₂—, —CH₂CH₂—, OCH₂—, and —CH₂CH₂CH₂—. In yet an even more specific aspect, L is chosen from a bond and —CH₂—. [symbol] xemplary compounds of formula (VI) include:

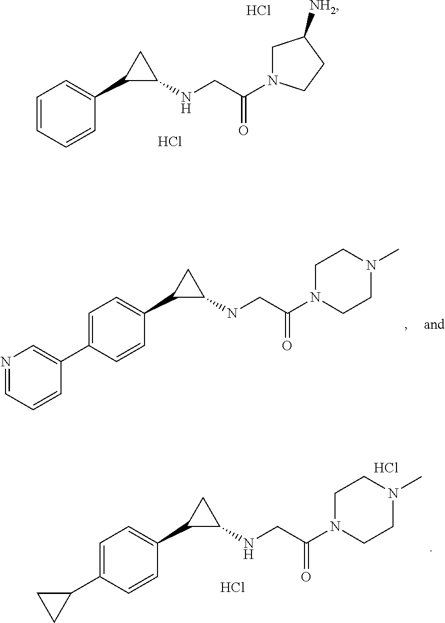

Exemplary compounds of formula (VI) include: N-cyclopropyl-2-{[(trans)-2-phenylcyclopropyl]amino}acetamide; 2-{[(trans)-2-phenylcyclopropyl]amino acetamide; N-cyclopropyl-2-{[(trans)-2-phenylcyclopropyl]amino}propanamide; 2-{[(trans)-2-phenylcyclopropyl]amino}-N-prop-2-ynylacetamide; N-isopropyl-2-{[(trans)-2-phenylcyclopropyl]amino}acetamide; N-(tert-butyl)-2-{[(trans)-2-phenylcyclopropyl]amino}acetamide; N-(2-morpholin-4-yl-2-oxoethyl)-N-[(trans)-2-phenylcyclopropyl]amine; 2-{[(trans)-2-phenylcyclopropyl]amino}propanamide; methyl 2-{[(trans)-2-phenylcyclopropyl]amino}propanoate; N-cyclopropyl-2-{methyl[(trans)-2-phenylcyclopropyl]amino}acetamide; 2-{methyl[(trans)-2-phenylcyclopropyl]amino}acetamide; N-methyl-trans-2-(phenylcyclopropylamino)propanamide; 1-(4-methylpiperazin-1-yl)-2-((trans)-2-phenylcyclopropylamino)ethanone; 1-(4-ethylpiperazin-1-yl)-2-((trans)-2-phenylcyclopropylamino)ethanone; 1-(4-benzylpiperazin-1-yl)-2-((trans)-2-phenylcyclopropylamino)-ethanone; 2-((trans)-2-phenylcyclopropylamino)-1-(4-phenylpiperazin-1-yl)ethanone; 2-((trans)-2-(4-(benzyloxy)phenyl)cyclopropylamino)-1-(4-methylpiperazin-1-yl)ethanone; 2-((trans)-2-(4-(benzyloxy)phenyl)cyclopropylamino)-N-cyclopropylacetamide; 2-((trans)-2-(4-(3-fluorobenzyloxy)phenyl)cyclopropylamino)-1-(4-methylpiperazin-1-yl)ethanone; 2-((trans)-2-(4-(3-chlorobenzyloxy)phenyl)cyclopropylamino)-1-(4-methylpi-perazin-1-yl)ethanone; 2-((trans)-2-(biphenyl-4-yl)cyclopropylamino)-1-(4-methylpiperazin-1-yl)ethanone; 1-(4-methylpiperazin-1-yl)-2-((trans)-2-(4-phenethoxyphenyl)cyclopropylamino)ethanone; 2-((trans)-2-(4-(4-fluorobenzyloxy)phenyl)cyclopropylamino)-1-(4-methylpi-perazin-1-yl)ethanone; 2-((trans)-2-(4-(biphenyl-4-ylmethoxy)phenyl)cyclopropylamino)-1-(4-methy-lpiperazin-1-yl)ethanone; (trans)-N-(4-fluorobenzyl)-2-phenylcyclopropanamine; (trans)-N-(4-fluorobenzyl)-2-phenylcyclopropanaminiurn; 4-(((trans)-2-phenylcyclopropylamino)methyl)benzonitrile; (trans)-N-(4-cyanobenzyl)-2-phenylcyclopropanaminium; (trans)-2-phenyl-N-(4-(trifluoromethyl)benzyl)cyclopropanamine; (trans)-2-phenyl-N-(4-(trifluoromethyl)benzyl)cyclopropanaminium; (trans)-2-phenyl-N-(pyridin-2- [symbol] panamine; (trans)-2-phenyl-N-(pyridin-3-ylmethyl)cyclo [symbol] )-2-phenyl-N-(pyridin-4-ylmethyl)cyclopropanamine; (trans)-N-((6-methylpyridin-2-yl)methyl)-2-phenylcyclopropanamine; (trans)-2-phenyl-N-(thiazol-2-ylmethyl)cyclopropanamine; (trans)-2-phenyl-N-(thiophen-2-ylmethyl)cyclopropanamine; (trans)-N-((3-bromothiophen-2-yl)methyl)-2-phenylcyclopropanamine; (trans)-N-((4-bromothiophen-2-yl)methyl)-2-phenylcyclopropanamine; (trans)-N-(3,4-dichlorobenzyl)-2-phenylcyclopropanamine; (trans)-N-(3-fluorobenzyl)-2-phenylcyclopropanaminium; (trans)-N-(2-fluorobenzyl)-2-phenylcyclopropanamine; (trans)-2-phenyl-N-(quinolin-4-ylmethyl)cyclopropanaraine; (trans)-N-(3-methoxybenzyl)-2-phenylcyclopropanamine; (trans)-2-phenyl-N-((6-(trifluoromethyl)pyridin-3-yl)methyl)cyclopropanamine; (trans)-N-((6-chloropyridin-3-yl)methyl)-2-phenylcyclopropanamine; (trans)-N-((4-methylpyridin-2-yl)methyl)-2-phenylcyclopropanamine; (trans)-N-((6-methoxypyridin-2-yl)methyl)-2-phenylcyclopropanamine; 2-(((trans)-2-phenylcyclopropylamino)methyl)pyridin-3-ol; (trans)-N-((6-bromopyridin-2-yl)methyl)-2-phenylcyclopropanamine; 4-(((trans)-2-(4-(benzyloxy)phenyl)cyclopropylamino)methyl)benzonitrile; (trans)-N-(4-(benzyloxy)benzyl)-2-phenylcyclopropanamine; (trans)-N-benzyl-2-(4-(benzyloxy)phenyl)cyclopropanamine; (trans)-2-(4-(benzyloxy)phenyl)-N-(4-methoxybenzyl)cyclopropanamine; (trans)-2-(4-(benzyloxy)phenyl)-N-(4-fluorobenzyl)cyclopropanamine-; (trans)-2-phenyl-N-(quinolin-2-ylmethyl)cyclopropanamine; (trans)-2-phenyl-N-((5-(trifluoromethyl)pyridin-2-yl)methyl)cyclopropanamine; (trans)-N-((3-fluoropyridin-2-yl)methyl)-2-phenylcyclopropanamine; (trans)-2-phenyl-N-(quinolin-3-ylmethyl)cyclopropanamine; (trans)-N-((6-methoxypyridin-3-yl)methyl)-2-phenylcyclopropanamine; (trans)-N-((5-methoxypyridin-3-yl)methyl)-2-phenylcyclopropanamine-; (trans)-N-((2-methoxypyridin-3-yl)methyl)-2-phenylcyclopropanamin-e; (trans)-N-((3H-indol-3-yl)methyl)-2-phenylcyclopropanamine; 3-(((trans)-2-phenylcyclopropylamino)methyl)benzonitrile; (trans)-N-(2-methoxybenzyl)-2-phenylcyclopropanamine; 3-(((trans)-2-phenylcyclopropylamino)methyl)pyridin-2-amine; (trans)-N-((2-chloropyridin-3-yl)methyl)-2-phenylcyclopropanamine; (trans)-N-(3,4-dimethoxybenzyl)-2-phenylcyclopropanamine; (trans)-N-((2,3-dihydrobenzofuran-5-yl)methyl)-2-phenylcyclopropanamine; (trans)-N-(benzo[d][1,3]dioxol-5-ylmethyl)-2-phenylcyclopropanamin-e; (trans)-N-((2,3-dihydrobenzo[b][1,4]dioxin-6-yl)methyl)-2-phenyl-cyclopropanamine; (trans)-N-(2,6-difluoro-4-methoxybenzyl)-2-phenylcyclopropanamine; (trans)-2-phenyl-N-(4-(trifluoromethoxy)benzyl)cyclopropanamine; (trans)-N-(5-fluoro-2-methoxybenzyl)-2-phenylcyclopropanamine; (trans)-N-(2-fluoro-4-methoxybenzyl)-2-phenylcyclopropanamine; (trans)-N-((4-methoxynaphthalen-1-yl)methyl)-2-phenylcyclopropanamine; (trans)-N-(2-fluoro-6-methoxybenzyl)-2-phenylcyclopropanamine; (trans)-N-((2-methoxynaphthalen-1-yl)methyl)-2-phenylcyclopropanamine; (trans)-N-((4,7-dimethoxynaphthalen-1-yl)methyl)-2-phenylcyclopropanamine-; (trans)-N-(4-methoxy-3-methylbenzyl)-2-phenylcyclopropanamine; (trans)-N-(3-chloro-4-methoxybenzyl)-2-phenylcyclopropanamine; (trans)-N-(3-fluoro-4-methoxybenzyl)-2-phenylcyclopropanamine; (trans)-N-(4-methoxy-2-methylbenzyl)-2-phenylcyclopropanamine; (trans)-N-((3,4-dihydro-2H-benzo[b][1,4]dioxepin-6-yl)methyl)-2-phenylcyclopropanamine; (trans)-N-((3,4-dihydro-2H-benzo[b][1,4]dioxepin-7-yl)methyl)-2-phenylcyclopropanamine; (trans)-N-((2,2-dimethylchroman-6-yl)methyl)-2-phenylcyclopropanamine; (trans)-N-(4-methoxy-2,3-dimethylbenzyl)-2-phenylcyclopropanamine; (trans)-N-(4-methoxy-2,5-dimethylbenzyl)-2-phenylcyclopropanamine; (trans)-N-(2-fluoro-4,5-dimethoxybenzyl)-2-phenylcyclopropanamine; (trans)-N-(3-chloro-4,5-dimethoxybenzyl)-2-phenylcyclopropanamine; (trans)-N-(2-chloro-3,4-dimethoxybenzyl)-2-phenylcyclopropanamine; (trans)-N-(2,4-dimethoxy-6-methylbenzyl)-2-phenylcyclopropanamine; [symbol] methoxybenzyl)-2-phenylcyclopropanamine; (trans)-N-( [symbol] )-2-phenylcyclopropanamine; (trans)-N-(2-chloro-3-methoxybenzyl)-2-phenylcyclopropanamine; (trans)-N-((1H-indol-5-yl)methyl)-2-phenylcyclopropanamine; (trans)-2-(4-(benzyloxy)phenyl)-N-(pyridin-2-ylmethyl)cyclopropanamine; (trans)-2-(4-(benzyloxy)phenyl)-N-(2-methoxybenzyl)cyclopropanamine; (trans)-N-(1-(4-methoxyphenyl)ethyl)-2-phenylcyclopropanaraine; (trans)-N-(1-(3,4-dimethoxyphenyl)ethyl)-2-phenylcyclopropanamine; (trans)-N-(1-(2,3-dihydrobenzo[b][1,4]dioxin-6-yl)ethyl)-2-phenylcyclopropanamine; (trans)-N-(1-(5-fluoro-2-methoxyphenyl)ethyl)-2-phenylcyclopropanamine; (trans)-N-(1-(3,4-dimethoxyphenyl)propan-2-yl)-2-phenylcyclopropan-amine; (trans)-N-((3-methyl-1,2,4-oxadiazol-5-yl)methyl)-2-phenylcyclopropanamine;

and pharmaceutically acceptable salts thereof.

Alternative small molecule LSD inhibitor compounds may be selected from selective LSD1 and LSD1/MAOB dual inhibitors disclosed for example in WO2010/043721 (PCT/EP2009/063685), WO2010/084160 (PCT/EP2010/050697), PCT/EP2010/055131; PCT/EP2010/055103; and EP application number EP10171345 all of which are explicitly incorporated herein by reference in their entireties to the extent they are not inconsistent with the instant disclosure. Representative compounds of this type include phenylcyclopropylamine derivatives or homologs, illustrative examples of which include phenylcyclopropylamine with one or two substitutions on the amine group; phenylcyclopropylamine with zero, one or two substitutions on the amine group and one, two, three, four, or five substitution on the phenyl group; phenylcyclopropylamine with one, two, three, four, or five substitution on the phenyl group; phenylcyclopropylamine with zero, one or two substitutions on the amine group wherein the phenyl group of PCPA is substituted with (exchanged for) another ring system chosen from aryl or heterocyclyl to give an aryl- or heteroaryl-cyclopropylamine having zero, one or two substituents on the amine group; phenylcyclopropylamine wherein the phenyl group of PCPA is substituted with (exchanged for) another ring system chosen from aryl or heterocyclyl to give an aryl- or heterocyclyl-cyclopropylamine wherein said aryl- or heterocyclyl-cyclopropylamine on said aryl or heterocyclyl moiety has zero, one or two substitutions on the amine group and one, two, three, four, or five substitution on the phenyl group; phenylcyclopropylamine with one, two, three, four, or five substitution on the phenyl group; or any of the above described phenylcyclopropylamine analogs or derivatives wherein the cyclopropyl has one, two, three or four additional substituents. Suitably, the heterocyclyl group described above in this paragraph in a heteroaryl.

Non-limiting embodiments of phenylcyclopropylamine derivatives or analogs include “cyclopropylamine amide” derivatives and “cyclopropylamine” derivatives. Specific examples of “cyclopropylamine acetamide” derivatives include, but are not limited to: N-cyclopropyl-2-{[(trans)-2-phenylcyclopropyl]amino}acetamide; 2-{[(trans)-2-phenylcyclopropyl]amino}acetamide; N-cyclopropyl-2-{[(trans)-2-phenylcyclopropyl]amino}propanamide; 2-{[(trans)-2-phenylcyclopropyl]amino}-N-prop-2-ynylacetamide; N-isopropyl-2-{[(trans)-2-phenylcyclopropyl]amino}acetamide; N-(tert-butyl)-2-{[(trans)-2-phenylcyclopropyl]amino}acetamide; N-(2-morpholin-4-yl-2-oxoethyl)-N-[(trans)-2-phenylcyclopropyl]amine; 2-{[(trans)-2-phenylcyclopropyl]amino}propanamide; Methyl 2-{[(trans)-2-phenylcyclopropyl]amino}propanoate; 1-(4-methylpiperazin-1-yl)-2-((trans)-2-phenylcyclopropylamino)ethanone; 1-(4-ethylpiperazin-1-yl)-2-((trans)-2-phenylcyclopropylamino)ethanone; 1-(4-benzylpiperazin-1-yl)-2-((trans)-2-phenylcyclopropylamino)ethanone; 2-((trans)-2-phenylcyclopropylamino)-1-(4-phenylpiperazin-1- [symbol] (trans)-2-(4-(benzyloxy)phenyl)cyclopropylamino)-1-(4-n [symbol] yl)ethanone; 2-((trans)-2-(1,1′-biphenyl-4-yl)cyclopropylamino)-1-(4-methylpiperazin-1-yl)ethanone; 2-((trans)-2-(4-(benzyloxy)phenyl)cyclopropylamino)-N-cyclopropylacetamide; 2-((trans)-2-(4-(3-fluorobenzyloxy)phenyl)cyclopropylamino)-1-(4-methylpiperazin-1-yl)ethanone; 2-((trans)-2-(4-(4-fluorobenzyloxy)phenyl)cyclopropylamino)-1-(4-methylpi-perazin-1-yl)ethanone; 2-((trans)-2-(4-(3-chlorobenzyloxy)phenyl)cyclopropylamino)-1-(4-methylpi-perazin-1-yl)ethanone; 1-(4-methylpiperazin-1-yl)-2-((trans)-2-(4-phenethoxyphenyl)cyclopropylamino)ethanone; 2-((trans)-2-(biphenyl-4-yl)cyclopropylamino)-1-(4-methylpiperazin-1-yl)ethanone; N-cyclopropyl-2-{[(trans)-2-phenylcyclopropyl]amino}acetamide; N-methyl-trans-2-(phenylcyclopropylamino)propanamide; 2-{methyl[(trans)-2-phenylcyclopropyl]amino}acetamide; N-[2-(4-methylpiperazin-1-yl)ethyl]-N-[(trans)-2-phenylcyclopropyl]amine; N-cyclopropyl-N′-[(trans)-2-phenylcyclopropyl]ethane-1,2-diamine; N,N-dimethyl-N′-(2-{[(trans)-2-phenylcyclopropyl]amino}ethyl)ethane-1,2-diamine; (3R)-1-(2-{[(trans)-2-phenylcyclopropyl]amino}ethyl)pyrrolidin-3-amine; (3S)-N,N-dimethyl-1-(2-{[(trans)-2-phenylcyclopropyl]amino}ethyl) pyrrolidin-3-amine; (3R)-N,N-dimethyl-1-(2-{[(trans)-2-phenylcyclopropyl]amino}ethyl) pyrrolidin-3-amine; N-[(trans)-2-phenylcyclopropyl]-N-(2-piperazin-1-ylethyl) amine; N,N-diethyl-N′-[(trans)-2-phenylcyclopropyl]ethane-1,2-diamine; N-[(trans)-2-phenylcyclopropyl]-N-(2-piperidin-1-ylethyl)amine; (trans)-2-(4-(benzyloxy)phenyl)-N-(2-(4-methylpiperazin-1-yl)ethyl)cyclopropanamine; (trans)-N-(2-(4-methylpiperazin-1-yl)ethyl)-2-(3′-(trifluoromethyl)biphenyl-4-yl)cyclopropanamine; (trans)-2-(3′-chlorobiphenyl-4-yl)-N-(2-(4-methylpiperazin-1-yl)ethyl)cyclopropanamine; (R)-1-(2-((trans)-2-(3′-(trifluoromethyl)biphenyl-4-yl)cyclopropylamino) ethyl)pyrrolidin-3-amine; and N¹-cyclopropyl-N²-((trans)-2-(3′-(trifluoromethyl)biphenyl-4-yl-)cyclopropyl)ethane-1,2-diamine.

Specific examples of “cyclopropylamine” derivatives, include, but are not limited to: N-4-fluorobenzyl-N-{(trans)-2-[4-(benzyloxy)phenyl]cyclopropyl}amine, N-4-methoxybenzyl-N-{(trans)-2-[4-(benzyloxy)phenyl]cyclopropyl}amine, N-benzyl-N-{(trans)-2-[4-(benzyloxy)phenyl]cyclopropyl}amine, N-[(trans)-2-phenylcyclopropyl]amino-methyl)pyridin-3-ol, N-[(trans)-2-phenylcyclopropyl]-N-(3-methylpyridin-2-ylmethyl)amine, N-[(trans)-2-phenylcyclopropyl]-N-(4-chloropyridin-3-ylmethyl)amine, N-[(trans)-2-phenylcyclopropyl]-N-(4-trifluoromethylpyridin-3-yl-methyl)amine, N-(3-methoxybenzyl)-N-[(trans)-2-phenylcyclopropyl]amine, N-[(trans)-2-phenylcyclopropyl]-N-(quinolin-4-ylmethyl)amine, N-(2-fluorobenzyl)-N-[(trans)-2-phenylcyclopropyl]amine, N-(3-fluorobenzyl)-N-[(trans)-2-phenylcyclopropyl]amine, N-[(trans)-2-phenylcyclopropyl]-N-(3,4-dichloro-1-phenylmethyl)amine, N-[(trans)-2-phenylcyclopropyl]-N-(5-bromo-thiophen-2-ylmethyl)amine, N-[(trans)-2-phenylcyclopropyl]-N-(3-bromo-thiophen-2-ylmethyl)-amine, N-[(trans)-2-phenylcyclopropyl]-N-(thiophen-2-ylmethyl)amine, N-[(trans)-2-phenylcyclopropyl]-N-(1,3-thiazol-2-ylmethyl)amine, N-[(trans)-2-phenylcyclopropyl]-N-(3-methyl-pyridin-2-ylmethyl)amine, N-[(trans)-2-phenylcyclopropyl]-N-(pyridin-4-ylmethyl)amine, N-[(trans)-2-phenylcyclopropyl]-N-(pyridin-3-ylmethyl)amine, N-[(trans)-2-phenylcyclopropyl]-N-(pyridin-2-ylmethyl)amine, [(trans)-2-phenylcyclopropyl]-N-[4-(trifluoromethyl)benzyl]amine, ({[(trans)-2-phenylcyclopropyl]amino}methyl)benzonitrile, N-(4-fluorobenzyl)-N-[(trans)-2-phenylcyclopropyl]amine, N-[(trans)-2-phenylcyclopropyl]-N-(3-bromo-pyridin-2-ylmethyl)amine, N-4-cyanobenzyl-N-{(trans)-2-[4-(benzyloxy)phenyl]cyclopropyl}amine, N-4-[(benzyloxy)-benzyl]-N-[(trans)-2-(4-phenyl)cyclopropyl]amine; 2-((trans)-2-(4-(4-cyanobenzyloxy)phenyl)cyclopropylamino)acetamide, 2-((trans)-2-(4-(3-cyanobenzyloxy)phenyl)cyclopropylamino)acetamide, 2-((trans)-2-(4- [symbol] yl)cyclopropylamino)acetamide, 2-((trans)-2-(4-(4-fluorobenzyloxy)phenyl)cyclopropylamino)acetamide, 2-((trans)-2-(4-(3-fluorobenzyloxy)phenyl)cyclopropylamino)acetamide, 2-((trans)-2-(4-(3-chlorobenzyloxy)phenyl)cyclopropylamino)acetamide, 2-((trans)-2-(4-(4-chlorobenzyloxy)phenyl)cyclopropylamino)acetamide, 2-((trans)-2-(4-(3-bromobenzyloxy)phenyl)cyclopropylamino)acetamide, 2-((trans)-2-(4-(3,5-difluorobenzyloxy)phenyl)cyclopropylamino)acetamide, 2-((trans)-2-(4-phenethoxyphenyl)cyclopropylamino)acetamide, 2-((trans)-2-(3′-(trifluoromethyl)biphenyl-4-yl)cyclopropylamino) acetamide, and 2-((trans)-2-(3′-chlorobiphenyl-4-yl)cyclopropylamino)acetamide.

Other examples of LSD1 inhibitors are, e.g., phenelzine or pargyline (propargylamine) or a derivative or analog thereof. Derivatives and analogs of phenelzine and pargyline (propargylamine) include, but are not limited to, compounds where the phenyl group of the parent compound is replaced with a heteroaryl or optionally substituted cyclic group or the phenyl group of the parent compound is optionally substituted with a cyclic group. In one aspect, the phenelzine or pargyline derivative or analog thereof has selective LSD1 or dual LSD1/MAOB inhibitory activity as described herein. In some embodiments, the phenelzine derivative or analog has one, two, three, four or five substituents on the phenyl group. In one aspect, the phenelzine derivative or analog has the phenyl group substituted with (exchanged for) an aryl or heterocyclyl group wherein said aryl or heterocyclyl group has zero, one, two, three, four or five substituents. In one aspect, the pargyline derivative or analog has one, two, three, four or five substituents on the phenyl group. In one aspect, the pargyline derivative or analog has the phenyl group substituted with (exchanged for) an aryl or heterocyclyl group wherein said aryl or heterocyclyl group has zero, one, two, three, four or five substituents. Methods of preparing such compounds are known to the skilled artisan.

The present invention also contemplates tranylcypromine derivatives as described for example by Binda et al. (2010. J. Am. Chem. Soc. 132:6827-6833, which is hereby incorporated by reference herein in its entirety) as inhibitors of LSD (e.g., LSD1 and/or LSD2) enzymatic function. Non-limiting example of such compounds include:

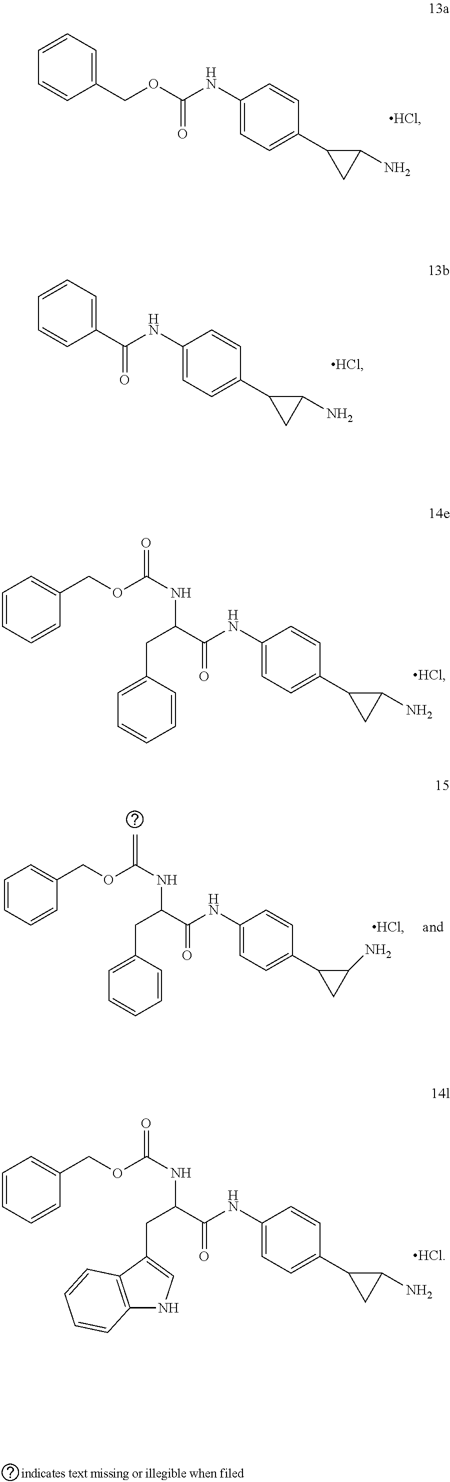 [SMILES: NC1CC1c1ccc(NC(=O)OCc2ccccc2)cc1 ; NC1CC1c1ccc(NC(=O)c2ccccc2)cc1 ; NC1CC1c1ccc(NC(=O)C(Cc2ccccc2)NC(=O)OCc2ccccc2)cc1 ; C=C(NC(Cc1ccccc1)C(=O)Nc1ccc(C2CC2N)cc1)OCc1ccccc1 ; NC1CC1c1ccc(NC(=O)C(Cc2c[nH]c3ccccc23)NC(=O)OCc2ccccc2)cc1]

Alternatively, LSD1 inhibitor compounds may be selected from tranylcypromine analogs described by Benelkebir et al. (2011. Bioorg. Med. Chem. doi:10.1016/j.bmc.2011.02.017, which is hereby incorporated by reference herein in its entirety). Representative analogs of this type, including o, m- and p-bromo analogues include: (1R,2S)-2-(4-bromophenyl)cyclopropanamine hydrochloride (Compound 4c), (1R,2S)-2-(3-bromophenyl)cyclopropanamine hydrochloride (Compound 4d), (1R,2S)-2-(2-bromophenyl)cyclopropanamine hydrochloride (Compound 4e), (1R,2S)-2-(biphenyl-4-yl)cyclopropanamine hydrochloride (Compound 4f).

Reference also may be made to peptide scaffold compounds disclosed by Culhane et al. (2010. J. Am. Chem. Soc. 132:3164-3176, which is hereby incorporated by reference herein in its entirety), which include chlorovinyl, endo-cyclopropylamine, and hydrazine functionalities. Non-limiting compounds disclosed by Culhane et al. include propargyl-Lys-4, N-methylpropargyl-Lys-4 H3-21, cis-3-chloroallyl-Lys-4 H3-21, trans-3-chloroallyl-Lys-4 H3-21, exo-cyclopropyl-Lys-4 H3-21, endo-cyclopropyl-Lys-4 H3-21, endo-dimethylcyclopropyl-Lys-4, hydrazino-Lys-4 H3-21 and hydrazino-Lys-4 H3-21.

Alternative cyclopropylamine compounds that are useful for inhibiting LSD1 include those disclosed by Fyfe et al. in U.S. Publication No. 2013/0197013, which is incorporated herein by reference in its entirety. Illustrative cyclopropylamine inhibitors of LSD1, which are disclosed as being selective for inhibiting LSD1, include compounds according to formula (VI)I:

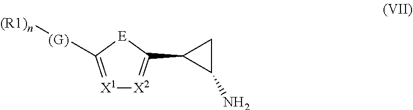

wherein:

E is —N(R3)-, —O—, or —S—, or is —X³═X⁴—;

X¹ and X² are independently C(R2) or N; [symbol] ³ and X⁴, when present, are independently C(R2) or N;

(G) is a cyclyl group (as shown in formula (VII), the cyclyl group (G) has n substituents (R₁));

each (R₁) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;

each (R₂) is independently chosen from —H, alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R₂) group has 1, 2, or 3 independently chosen optional substituents or two (R₂) groups can be taken together to form a heterocyclyl or aryl group having 1, 2, or 3 independently chosen optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;

R₃ is —H or a (C₁-C₆)alkyl group;

each L1 is independently alkylene or heteroalkylene; and

n is 0, 1, 2, 3, 4 or 5,

or an enantiomer, a diastereomer, or a mixture thereof, or a pharmaceutically acceptable salt or solvate thereof.

In some embodiments, compounds of formula (VII) are represented by formula (VIII):

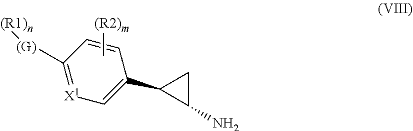

wherein:

X¹ is CH or N; (G) is a cyclyl group;

each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;

each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate; [symbol] ach L1 is independently alkylene or heteroalkylene;

m is 0, 1, 2 or 3; and n is 0, 1, 2, 3, 4 or 5, provided that n and m are chosen independently such that n+m is greater than zero when X¹ is —CH— and (G) is an aryl,

or an enantiomer, a diastereomer, or a mixture thereof, or a pharmaceutically acceptable salt or solvate thereof.

In other embodiments, compounds of formula (VII) are represented by formula (IX):

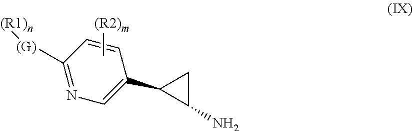

wherein:

(G) is a cyclyl group;

each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;

each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 0, 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;

each L1 is independently alkylene or heteroalkylene; m is 0, 1, 2 or 3; and

n is 0, 1, 2, 3, 4 or 5,

or an enantiomer, a diastereomer, or a mixture thereof, or a pharmaceutically acceptable salt or solvate thereof.

In still other embodiments, compounds of formula (VII) are represented by formula (X):

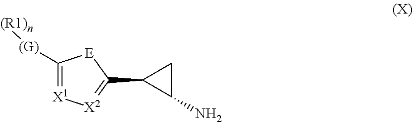

wherein:

E is —N(R3)-, —O—, or —S—, or is —X³═X⁴—;

X¹, X², X³ and X⁴ are independently C(R2) or N, provided that at least one of X¹, X², X³ and X⁴ is N when E is —X³═X⁴—; [symbol] (G) is a cyclyl group; each (R1) is independently chosen fr [symbol] alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;

each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;

R3 is —H or a (C₁-C₆)alkyl group; each L1 is alkylene or heteroalkylene; and n is 0, 1, 2, 3, 4 or 5,

or an enantiomer, a diastereomer, or a mixture thereof, or a pharmaceutically acceptable salt or solvate thereof.

In still other embodiments, compounds of formula (VII) are represented by formula (XI):

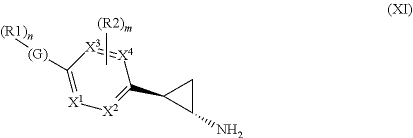

wherein:

X¹, X², X³ and X⁴ are independently CH or N, provided that at least one of X¹, X², X³ and X⁴ is N;

(G) is a cyclyl group; each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;

each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate; each L1 is alkylene or heteroalkylene;

m is 0, 1, 2 or 3; and n is 0, 1, 2, 3, 4 or 5, [symbol] an enantiomer, a diastereomer, or a mixture thereof, or [symbol] acceptable salt or solvate thereof.

Representative compounds according to formula (VII) are suitably selected from: (trans)-2-(3′-(trifluoromethyl)biphenyl-4-yl)cyclopropanamine; (trans)-2-(terphenyl-4-yl)cyclopropanamine; 4′-((trans)-2-aminocyclopropyl)biphenyl-4-ol; 4′-((trans)-2-aminocyclopropyl)biphenyl-3-ol; (trans)-2-(6-(3-(trifluoromethyl)phenyl)pyridin-3-yl)cyclopropanamine; (Trans)-2-(6-(3,5-dichlorophenyl)pyridin-3-yl)cyclopropanamine; (trans)-2-(6-(4-chlorophenyl)pyridin-3-yl)cyclopropanamine; (trans)-2-(6-(3-chlorophenyl)pyridin-3-yl)cyclopropanamine; (trans)-2-(6-(4-(trifluoromethyl)phenyl)pyridin-3-yl)cyclopropanamine; (trans)-2-(6-(4-methoxyphenyl)pyridin-3-yl)cyclopropanamine; (trans)-2-(6-(3-methoxyphenyl)pyridin-3-yl)cyclopropanamine; 4-(5-((trans)-2-aminocyclopropyl)pyridin-2-yl)benzonitrile; 3-(5-((trans)-2-aminocyclopropyl)pyridin-2-yl)benzonitrile; (Trans)-2-(6-p-tolylpyridin-3-yl)cyclopropanamine; (Trans)-2-(6-m-tolylpyridin-3-yl)cyclopropanamine; 4-(5-((trans)-2-aminocyclopropyl)pyridin-2-yl)phenol; 3-(5-((trans)-2-aminocyclopropyl)pyridin-2-yl)phenol; 4-(5-((trans)-2-aminocyclopropyl)pyridin-2-yl)benzamide; 3-(5-((trans)-2-aminocyclopropyl)pyridin-2-yl)benzamide; 2-(5-((trans)-2-aminocyclopropyl)pyridin-2-yl)phenol; 3-(5-((trans)-2-aminocyclopropyl)pyridin-2-yl)phenol; (Trans)-2-(6-(3-methoxy-4-methylphenyl)pyridin-3-yl)cyclopropanamine; 5-(5-((trans)-2-aminocyclopropyl)pyridin-2-yl)-2-fluorophenol; 3-(5-((trans)-2-aminocyclopropyl)pyridin-2-yl)-5-fluorophenol; 3-(5-((trans)-2-aminocyclopropyl)pyridin-2-yl)-4-fluorophenol; 3-(5-((trans)-2-aminocyclopropyl)pyridin-2-yl)-2-fluorophenol; 3-(5-((trans)-2-aminocyclopropyl)pyridin-2-yl)-2,4-difluorophenol; 3-(5-((trans)-2-aminocyclopropyl)pyridin-2-yl)-2,4,6-trifluorophenol; 3-(5-((trans)-2-aminocyclopropyl)pyridin-2-yl)-5-chlorophenol; (Trans)-2-(6-(2-fluoro-3-(trifluoromethyl)phenyl)pyridin-3-yl)cyclopropanamine; (Trans)-2-(6-(5-chlorothiophen-2-yl)pyridin-3-yl)cyclopropanamine; (Trans)-2-(6-(5-methylthiophen-2-yl)pyridin-3-yl)cyclopropanamine; (Trans)-2-(6-(1H-indol-6-yl)pyridin-3-yl)cyclopropanamine; (Trans)-2-(6-(benzo[b]thiophen-5-yl)pyridin-3-yl)cyclopropanamine; 3-(5-((trans)-2-aminocyclopropyl)-3-methylpyridin-2-yl)phenol; (trans)-2-(6-(3-chlorophenyl)-5-methylpyridin-3-yl)cyclopropanamine; (trans)-2-(5-methyl-6-(3-(trifluoromethyl)phenyl)pyridin-3-yl)cyclopropanamine; (trans)-2-(6-(4-fluoro-3-methoxyphenyl)pyridin-3-yl)cyclopropanamine, (trans)-2-(6-(3-fluoro-5-methoxyphenyl)pyridin-3-yl)cyclopropanamine; (trans)-2-(6-(2-fluoro-5-methoxyphenyl)pyridin-3-yl)cyclopropanamine, (trans)-2-(6-(2-fluoro-3-methoxyphenyl)pyridin-3-yl)cyclopropanamine; (trans)-2-(6-(3-chloro-5-methoxyphenyl)pyridin-3-yl)cyclopropanamine; (trans)-2-(6-(2-chloro-5-methoxyphenyl)pyridin-3-yl)cyclopropanamine; (trans)-2-(6-(3-methoxy-5-(trifluoromethyl)phenyl)pyridin-3-yl)cyclopropanamine; 3-(5-((trans)-2-aminocyclopropyl)pyridin-2-yl)-5-methoxybenzonitri-le; 5-(5-((trans)-2-aminocyclopropyl)pyridin-2-yl)-2-methylphenol; 3-(5-((trans)-2-aminocyclopropyl)pyridin-2-yl)-4-chlorophenol; 3-(5-((trans)-2-aminocyclopropyl)pyridin-2-yl)-5-(trifluoromethyl)phenol; (trans)-2-(6-(2-fluoro-5-(trifluoromethyl)phenyl)pyridin-3-yl)cyclopropanamine; (trans)-2-(6-(2-chloro-5-(trifluoromethyl)phenyl)pyridin-3-yl)cyclopropanamine; (trans)-2-(6-(3,5-bis(trifluoromethyl)phenyl)pyridin-3-yl)cyclopropanamine; N-(3-(5-((trans)-2-aminocyclopropyl)pyridin-2-yl)phenyl)acetamide; N-(3-(5-((trans)-2-aminocyclopropyl)pyridin-2-yl)phenyl)methanesulfonamide; (trans)-2-(6-(benzo[b]thiophen-2-yl)pyridin-3-yl)cyclopropanamine; (trans)-2-(6-(benzo[b]thiophen-3-yl)pyridin-3-yl)cyclopropanamine; 5-(5-((trans)-2-aminocyclopropyl)pyridin-2-yl)thiophene-2-carbonitrile; (trans)-2-(6-(4-methylthiophen-3-yl)pyridin-3-yl)cyclopropanamine; (trans)-2-(2-chloro-6-(3-(trifluoromethyl)phenyl)pyridin-3-yl)cyclopropanamine; (trans)-2-(2-(4-chlorophenyl)-6-(3- [symbol] phenyl)pyridine-3-yl)cyclopropanamine; 4-(3-((trans)-2- [symbol] -(3-(trifluoromethyl)phenyl)pyridin-2-yl)phenol; 4-(3-((trans)-2-aminocyclopropyl)-6-(3-(trifluoromethyl)phenyl)-pyridin-2-yl)benzamide; (trans)-2-(2-methyl-6-(3-(trifluoromethyl)phenyl)pyridin-3-yl)cyclopropanamine; 3-(5-((trans)-2-aminocyclopropyl)pyridin-2-yl)-5-hydroxybenzonitril-e; (trans)-2-(6-(3,4-difluoro-5-methoxyphenyl)pyridin-3-yl)cyclopropanamine; 5-(5-((trans)-2-aminocyclopropyl)pyridin-2-yl)-2,3-difluorophenol; (trans)-2-(6-(3-chloro-4-fluoro-5-methoxyphenyl)pyridin-3-yl)cyclopropanamine; 5-(5-((trans)-2-aminocyclopropyl)pyridin-2-yl)-3-chloro-2-fluorophen-ol; (trans)-2-(6-(1H-indazol-6-yl)pyridin-3-yl)cyclopropanamine; (trans)-2-(6-(9H-carbazol-2-yl)pyridin-3-yl)cyclopropanamine; 6-(5-((trans)-2-aminocyclopropyl)pyridin-2-yl)indolin-2-one; 6-(5-((trans)-2-aminocyclopropyl)pyridin-2-yl)benzofuran-2(3H)-one; 4-(5-((trans)-2-aminocyclopropyl)pyridin-2-yl)pyridin-2(1H)-one; N-(3-(5-((trans)-2-aminocyclopropyl)pyridin-2-yl)phenyl)benzenesulfonamide; N-(3-(5-((trans)-2-aminocyclopropyl)pyridin-2-yl)phenyl)propane-2-sulfonamide; 4′-((trans)-2-aminocyclopropyl)-4-fluorobiphenyl-3-ol; 4′-((trans)-2-aminocyclopropyl)-5-chlorobiphenyl-3-ol; 4′-((trans)-2-aminocyclopropyl)-5-chloro-4-fluorobiphenyl-3-ol; N-(4′-((trans)-2-aminocyclopropyl)biphenyl-3-yl)benzenesulfonamide; N-(4′-((trans)-2-aminocyclopropyl)biphenyl-3-yl)propane-2-sulfonamide; N-(4′-((trans)-2-aminocyclopropyl)biphenyl-3-yl)methanesulfonamide; N-(2-(5-((trans)-2-aminocyclopropyl)pyridin-2-yl)phenyl)methanesulfonamide; 3-(5-((trans)-2-aminocyclopropyl)pyridin-2-yl)-4-methoxybenzonitrile; N-(4′-((trans)-2-aminocyclopropyl)biphenyl-2-yl)methanesulfonamide; 4′-((trans)-2-aminocyclopropyl)-6-methoxybiphenyl-3-carbonitrile; N-(4′-((trans)-2-aminocyclopropyl)-6-methoxybiphenyl-3-yl)methanesulfonamide; 4′-((trans)-2-aminocyclopropyl)-6-hydroxybiphenyl-3-carbonitrile; N-(4′-((trans)-2-aminocyclopropyl)-6-hydroxybiphenyl-3-yl)methanesulfonamide; 3-(5-((trans)-2-aminocyclopropyl)pyridin-2-yl)-4-hydroxybenzonitrile; N-(3-(5-((trans)-2-aminocyclopropyl)pyridin-2-yl)-4-hydroxyphenyl)methane-sulfonamide; N-(3-(5-((trans)-2-aminocyclopropyl)pyridin-2-yl)-5-(trifluoromethyl)phenyl)ethanesulfonamide; N-(3-(5-((trans)-2-aminocyclopropyl)pyridin-2-yl)-5-(trifluoromethyl)phenyl)methanesulfonamide; 3-(6-((trans)-2-aminocyclopropyl)pyridin-3-yl)phenol; (Trans)-2-(5-(3-methoxyphenyl)pyridin-2-yl)cyclopropanamine; 4-(6-((trans)-2-aminocyclopropyl)pyridin-3-yl)phenol; 2-(6-((trans)-2-aminocyclopropyl)pyridin-3-yl)phenol; 2-(5-((trans)-2-aminocyclopropyl)thiophen-2-yl)phenol; 3-(5-((trans)-2-aminocyclopropyl)thiophen-2-yl)phenol; 4-(5-((trans)-2-aminocyclopropyl)thiophen-2-yl)phenol; 2-(5-((trans)-2-aminocyclopropyl)thiazol-2-yl)phenol; 3-(5-((trans)-2-aminocyclopropyl)thiazol-2-yl)phenol; 4-(5-((trans)-2-aminocyclopropyl)thiazol-2-yl)phenol; 2-(2-((trans)-2-aminocyclopropyl)thiazol-5-yl)phenol; 3-(2-((trans)-2-aminocyclopropyl)thiazol-5-yl)phenol; 2-(2-((trans)-2-aminocyclopropyl)thiazol-5-yl)phenol; 3-(2-((trans)-2-aminocyclopropyl)thiazol-5-yl)phenol; 3-(5-((trans)-2-aminocyclopropyl)pyrimidin-2-yl)phenol; 4-(5-((trans)-2-aminocyclopropyl)pyrimidin-2-yl)phenol; N-(3-(5-((trans)-2-aminocyclopropyl)pyridin-2-yl)-4-methoxyphenyl)methane-sulfonamide; N-(4′-((trans)-2-aminocyclopropyl)-5-chloro-[1,1′-biphenyl]-3-yl)methanesulfonamide; N-(3-(5-((trans)-2-aminocyclopropyl)pyridin-2-yl)-5-chlorophenyl)methanesulfonamide; N-(4′-((trans)-2-aminocyclopropyl)-4-fluoro-[1,1′-biphenyl]-3-yl)methanesulfonamide; N-(5-(5-((trans)-2-aminocyclopropyl)pyridin-2-yl)-2-fluorophenyl)methanesulfonamide; N-(3-(5-((trans)-2-aminocyclopropyl)pyridin-2-yl)phenyl)ethanesulfonamide-; N-(3-(5-((trans)-2-aminocyclopropyl)pyridin-2-yl)phenyl)-4-cyanobenzenesulfonamide; N-(3-(5-((trans)-2-aminocyclopropyl)pyridin-2-yl)phenyl)-3-cyanobenzenesulfonamide; N-(3-(5-((trans)-2-aminocyclopropyl)pyridin-2-yl)phenyl)-2-cyanobenzenesulfonamide; N-(3-(5-((trans)-2- [symbol] )pyridin-2-yl)-5-(trifluoromethyl)phenyl)-4-cyanobenzen [symbol] ((trans)-2-aminocyclopropyl)-[1,1′-biphenyl]-3-yl)-1,1,1-trifluorom-ethanesulfonamide; 4′-((trans)-2-aminocyclopropyl)-6-hydroxy-[1,1′-biphenyl]-3-carbonitrile; 4′-((trans)-2-aminocyclopropyl)-[1,1′-biphenyl]-2-ol, 4′-((trans)-2-aminocyclopropyl)-3′-methoxy-[1,1′-biphenyl]-3-ol; N-(3-(5-((trans)-2-aminocyclopropyl)thiazol-2-yl)phenyl)-2-cyanobenzenesulfonamide; or a pharmaceutically acceptable salt or solvate thereof.

In other embodiments, LSD1 inhibitor compounds are selected from phenylcyclopropylamine derivatives, as described for example by Ogasawara et al. (2013, Angew. Chem. Int. Ed. 52:8620-8624, which is hereby incorporated by reference herein in its entirety). Representative compounds of this type are represented by formula (XII):

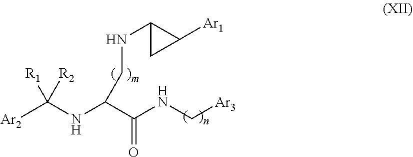

wherein Ar₁ is a 5 to 7 membered aryl or heteroaryl ring;

Ar₂ and Ar₃ are each independently selected from a 5 to 7 membered aryl or heteroaryl ring, optionally substituted with 1 to 3 substituents;

R₁ and R₂ are independently selected from hydrogen and hydroxyl or taken together R₁ and R₂ form ═O, ═S or ═NR₃;

R₃ is selected from hydrogen, —C₁₋₆ alkyl or —OH;

m is an integer from 1 to 5; and

n is an integer from 1 to 3;

or a pharmaceutically acceptable salt thereof.

In particular embodiments of formula (XII), one or more of the following applies:

Ar₁ is a six membered aryl or heteroaryl ring, especially phenyl, pyridine, pyrimidine, pyrazine 1,3,5-triazine, 1,2,4-trazine and 1,2,3-triazine, more especially phenyl;

Ar₂ is a six membered aryl or heteroaryl ring, especially phenyl, pyridine, pyrimidine, pyrazine 1,3,5-triazine, 1,2,4-trazine and 1,2,3-triazine, especially phenyl; especially where the six membered aryl or heteroaryl ring is optionally substituted with one optional substituent, especially in the 3 or 4 position;

Ar₃ is a six membered aryl or heteroaryl ring, especially phenyl, pyridine, pyrimidine, pyrazine 1,3,5-triazine, 1,2,4-trazine and 1,2,3-triazine, especially phenyl; especially where the six membered aryl or heteroaryl ring is optionally substituted with one optional substituent, especially in the 3 or 4 position.

Particular optional substituents for Ar₁ and Ar₂ include —C₁₋₆ alkyl, —C₂₋₆alkenyl, —CH₂F, —CHF₂, —CF₃, halo, aryl, heteroaryl, —C(O)NHC₁₋₆alkyl, —C(O)NHC₁₋₆alkylNH₂, —C(O)— heterocyclyl, especially methyl, ethyl, propyl, butyl, t-butyl, —CH₂F, —CHF₂, —CH₃, Cl, F, phenyl, —C(O)NH(CH₂)₁₋₄NH₂ and —C(O)-heterocyclyl; [symbol] and R₂ taken together form ═O, ═S or ═NR₃, especially [symbol] especially ═O;

R₃ is H, —C₁₋₃alkyl or —OH, especially H, —CH₃ or —OH.

m is 2 to 5, especially 3 to 5, more especially 4,

n is 1 or 2, especially 1.

In some embodiments the compounds of formula (XII) are compounds of formula (XIIa):

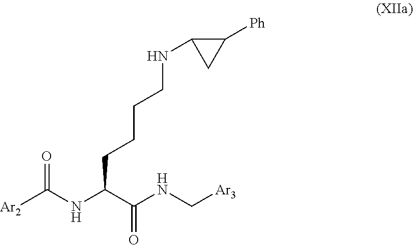

wherein Ar₂ and Ar₃ are as defined for formula (XII).

Non-limiting compounds represented by formula (XII) include the following:

| Compound | Ar₂ | Ar₃ |
|---|---|---|
| 1b | phenyl | phenyl |
| 1c | 4-nnethylphenyl | phenyl |
| 1d | 4-t-butylphenyl | phenyl |
| 1e | 4-chlorophenyl | phenyl |
| 1f | 4-fluorophenyl | phenyl |
| 1g | 4-phenyl-phenyl | Phenyl |
| 1h | 4-trifluoromethylphenyl | Phenyl |
| 1i | 3-(2-aminoethylcarbamoyl)phenyl | Phenyl |
| 1j | 3-(piperazine-l-carbonyl)phenyl | Phenyl |
| 1k | 4-phenyl-phenyl | 4-methylphenyl |
| 1l | 4-phenyl-phenyl | 4-fluorophenyl |
| 1m | 4-phenyl-phenyl | 4-phenyl-phenyl |
| 1n | 4-phenyl-phenyl | 4-t-butylphenyl |
| 1o | 4-phenyl-phenyl | 3-methylphenyl |
| 1p | 4-phenyl-phenyl | 3-fluorophenyl |
| 1q | 4-phenyl-phenyl | 3-phenyl-phenyl |

An exemplary compound according to formula (XII) (designated NCD-38 herein) is represented by the following structure:

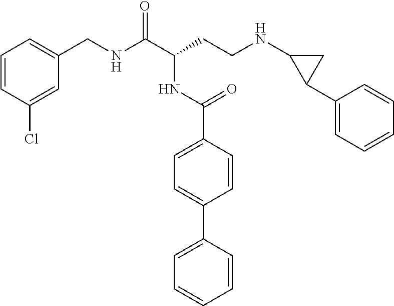 [SMILES: O=C(N[C@@H](CCNC1CC1c1ccccc1)C(=O)NCc1cccc(Cl)c1)c1ccc(-c2ccccc2)cc1]

The synthesis and inhibitory activity of the compounds of formula (VII) are described by Ogasawara et al. (2013, supra).

Other LSD1 inhibitors include, but are not limited to those, e.g., disclosed in Ueda et al. (2009. J. Am. Chem. Soc. 131(48):17536-17537) including; Mimasu i (2010. Biochemistry June 22. [Epub ahead of print] PMID: 20568732 [PubMed—as supplied by publisher].

Other phenylcyclopropylamine derivatives and analogs are found, e.g., in Kaiser et al. (1962, J. Med. Chem. 5:1243-1265); Zirkle et al. (1962. J. Med. Chem. 1265-1284; U.S. Pat. Nos. 3,365,458; 3,471,522; 3,532,749) and Bolesov et al. (1974. Zhurnal Organicheskoi Khimii 10:8 1661-1669) and Russian Patent No. 230169 (19681030).

In other embodiments, LSD1 inhibitor compounds are selected from cyclopropaneamine compounds, as described for example by Tomita et al. in U.S. Publication No. 2014/0228405, which is hereby incorporated by reference herein in its entirety. Representative compounds of this type are represented by formula (XIII):

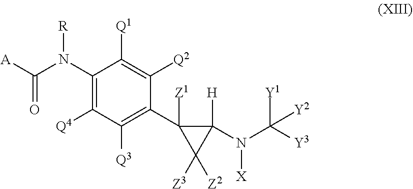

wherein:

A is a hydrocarbon group optionally having substituent(s), or a heterocyclic group optionally having substituent(s);

R is a hydrogen atom, a hydrocarbon group optionally having substituent(s), or a heterocyclic group optionally having substituent(s); or

A and R are optionally bonded to each other to form a ring optionally having substituent(s);

Q¹, Q², Q³ and Q⁴ are each independently a hydrogen atom or a substituent; Q¹ and Q², and Q³ and Q⁴, are each optionally bonded to each other to form a ring optionally having substituent(s); [symbol] is a hydrogen atom, an acyclic hydrocarbon group optio [symbol] substituent(s), or a saturated cyclic group optionally having substituent(s);

Y¹, Y² and Y³ are each independently a hydrogen atom, a hydrocarbon group optionally having substituent(s), or a heterocyclic group optionally having substituent(s);

X and Y¹, and Y¹ and Y², are each optionally bonded to each other to form a ring optionally having substituent(s); and

Z¹, Z² and Z³ are each independently a hydrogen atom or a substituent, or a salt thereof.

In specific embodiments of compounds according to formula (XIII), A is a phenyl group optionally having 1 to 3 C₁₋₆ alkyl groups substituted by 1 to 3 halogen atoms, a biphenylyl group, or a pyrazolyl group; R is a hydrogen atom; or A and R are optionally bonded to each other to form a dihydroisoindole ring having 1 or 2 oxo groups; Q¹ is a hydrogen atom or a C₁₋₆ alkyl group; Q², Q³ and Q⁴ are each a hydrogen atom; X is a hydrogen atom; Y¹, Y² and Y³ are each independently a hydrogen atom or a C₃-8 cycloalkyl group; Y¹ and Y¹ are optionally bonded to each other to form, together with the adjacent carbon atom, a piperidine ring optionally having 1 to 3 C₁₋₆ alkyl groups; and Z¹, Z² and Z³ are each a hydrogen atom, or a salt thereof.

Representative compounds according to formula (XIII) are suitably selected from: (1) N-(4-{trans-2-[(cyclopropylmethyl)amino]cyclopropyl}-2-methylphenyl)benzamide, (2) N-(4-{trans-2-[(cyclopropylmethyl)amino]cyclopropyl}phenyl)-3-(trifluoromethoxy)benzamide, (3) N-(4-{trans-2-[(cyclopropylmethyl)amino]cyclopropyl}phenyl)benzamide, (4) N-(4-{trans-2-[(cyclopropylmethyl)amino]cyclopropyl}phenyl)-cyclohexanecarboxamide, (5) N-(4-{trans-2-[(1,1-dioxidotetrahydro-2H-thiopyran-4-yl)amino]cyclopropyl-}phenyl)-3-(trifluoromethyl)benzamide, (6) N-(4-{trans-2-[(cyclopropylmethyl)amino]cyclopropyl}phenyl)-1,3-dimethyl-1H-pyrazole-5-carboxamide, (7) N-(4-{trans-2-[(cyclopropylmethyl)amino]cyclopropyl}phenyl)-1,5-dimethyl-1H-pyrazole-3-carboxamide, (8) N-(4-{trans-2-[(cyclopropylmethyl)amino]cyclopropyl}phenyl)-1-methyl-3-(trifluoromethyl)-1H-pyrazole-4-carboxamide, (9) N-(4-{trans-2-[(cyclopropylmethyl)amino]cyclopropyl}phenyl)-1-methyl-5-(trifluoromethyl)-1H-pyrazole-4-carboxamide, and (10) N-(4-{trans-2-[(cyclopropylmethyl)amino]cyclopropyl}phenyl)-1-methyl-1H-pyrazole-4-carboxamide, or a salt thereof.

In still other embodiments, LSD1 inhibitor compounds are selected from compounds described for example by Munoz et al. in U.S. Publication No. 2014/0213657, which is hereby incorporated by reference herein in its entirety. Representative compounds of this type are represented by formula (XIV):

(A′)ₓ-(A)-(B)-(Z)-(L)-(D) (XIV)

wherein:

(A) is heteroaryl or aryl;

each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, haloalkoxy, aryl, arylalkoxy, alkyl, alkoxy, amido, —CH₂C(═O)NH₂, heteroaryl, cyano, sulfonyl, and sulfinyl;
X is 0, 1, 2, or 3; [symbol] ) is a cyclopropyl ring, wherein (A) and (Z) are covalent [symbol] carbon atoms of (B);

(Z) is —NH—; (L) is chosen from a single bond, —CH₂—, —CH₂CH₂—, —CH₂CH₂CH₂—, and —CH₂CH₂CH₂CH₂—; and

(D) is an aliphatic carbocyclic group or benzocycloalkyl, wherein said aliphatic carbocyclic group or said benzocycloalkyl has 0, 1, 2, or 3 substituents independently chosen from —NH₂, —NH(C₁-C₅ alkyl), —N(C₁-C₆ alkyl)(C₁-C₆ alkyl), alkyl, halo, amido, cyano, alkoxy, haloalkyl, and haloalkoxy; or an enantiomer, a diastereomer, or a mixture thereof, or a pharmaceutically acceptable salt or solvate thereof.

Non-limiting examples of compounds according to formula (XIV) include N-((trans)-2-(4-(benzyloxy)phenyl)cyclopropyl)-6-methoxy-2,3-dihydro-1H-i-nden-1-amine; N-((trans)-2-(4-(benzyloxy)phenyl)cyclopropyl)-5,6-dimethoxy-2,3-dihydro-1H-inden-1-amine; N-((trans)-2-(4-(benzyloxy)phenyl)cyclopropyl)-4,5-dimethoxy-2,3-dihydro-1H-inden-1-amine; N-((trans)-2-phenylcyclopropyl)-2,3-dihydro-1H-inden-1-amine; 6-methoxy-N-((trans)-2-phenylcyclopropyl)-2,3-dihydro-1H-inden-1-amine; 6-chloro-N-((trans)-2-phenylcyclopropyl)-2,3-dihydro-1H-inden-1-amine; N-((trans)-2-phenylcyclopropyl)-6-(trifluoromethyl)-2,3-dihydro-1H-inden-1-amine; 7-methoxy-N-((trans)-2-phenylcyclopropyl)-1,2,3,4-tetrahydronaphthalen-1-amine; N-((trans)-2-(3′-chlorobiphenyl-4-yl)cyclopropyl)-6-methoxy-2,3-dihydro-1-H-inden-1-amine; N-((trans)-2-(4′-chlorobiphenyl-4-yl)cyclopropyl)-6-methoxy-2,3-dihydro-1-H-inden-1-amine; 6-methoxy-N-((trans)-2-(3′-methoxybiphenyl-4-yl)cyclopropyl)-2,3-dihydro-1H-inden-1-amine; N-trans-(2-cyclohexylethyl)-2-phenylcyclopropanamine; (Trans)-N-(3-cyclohexylpropyl)-2-phenylcyclopropanamine; (Trans)-N-(2-cycloheptylethyl)-2-phenylcyclopropanamine; (Trans)-2-(4-(3-bromobenzyloxy)phenyl)-N-(2-cyclohexylethyl)cyclopropanamine; N-((trans)-2-(4-(3-bromobenzyloxy)phenyl)cyclopropyl)-6-methoxy-2,3-d-ihydro-1H-inden-1-amine; (Trans)-2-(3′-chlorobiphenyl-4-yl)-N-(2-cyclohexylethyl)cyclopropanamine; (Trans)-2-(4′-chlorobiphenyl-4-yl)-N-(2-cyclohexylethyl)cyclopropanamine; (Trans)-N-(2-cyclohexylethyl)-2-(3′-methoxybiphenyl-4-yl)cyclopropanamine-; N-((trans)-2-(4-(benzyloxy)phenyl)cyclopropyl)-7-methoxy-1,2,3,4-tetrahydronaphthalen-1-amine; and 1-((trans)-2-(4-(benzyloxy)phenyl)cyclopropylamino)cyclopropanecarboxamide; or a pharmaceutically acceptable salt or solvate thereof.

In still other embodiments, LSD1 inhibitor compounds are selected from substituted (E)-N′-(1-phenylethylidene)benzohydrazide analogs, as described for example by Vankayalapati et al. in U.S. Publication No. 2014/0163017, which is hereby incorporated by reference herein in its entirety. Representative compounds of this type are represented by formula (XV):

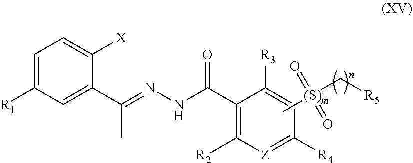

or by formula (XVI):

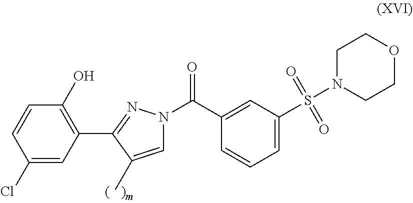

wherein:

m is 0 or 1;

n is an integer from 0 to 3;

X is selected from the group consisting of OH, NO₂ and F;

Z is selected from the group consisting of N and CH;

R₁ is selected from the group consisting of halo, C₁-C₃ haloalkyl, and C₁-C₃ polyhaloalkyl;

each of R₂, R₃, and R₄ is independently selected from the group consisting of hydrogen, halo, hydroxyl, cyano, amino, C₂-C₆ alkalkoxy, C₁-C₆ alkoxy, C₁-C₆ alkyl, C₁-C₆ polyhaloalkyl, and C₁-C₆ haloalkyl;

R₅ is selected from the group consisting of NR₆R₇, C₁-C₆ alkyl, C₃-C₆ cycloalkyl,

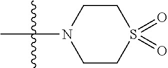 [SMILES: *N1CCS(=O)(=O)CC1]

and Cy, and substituted with 0-3 groups independently selected from halo, hydroxyl, amino, C₂-C₆ alkalkoxy, C₁-C₆ alkylalcohol, C₁-C₆ alkoxy, C₁-C₆ alkyl, C₁-C₆ polyhaloalkyl, C₁-C₆ haloalkyl, C₃-C₆ cycloalkyl, and Cy; Cy is a heterocycloalkyl selected from the group consisting of aziridinyl, azetidinyl, pyrrolidinyl, piperidinyl, azepanyl, oxazolidinyl, imidazolidinyl, pyrazolidinyl, piperazinyl, oxazinanyl, morpholinyl, hexahydrophyrimidinyl, and hexahydropyridazinyl; and each of R₆ and R₇ is independently selected from the group consisting of hydrogen, C₁-C₆ alkyl, C₃-C₆ cycloalkyl, and C₃-C₆ heterocycloalkyl; or a pharmaceutically acceptable salt thereof.

Illustrative compounds according to formulas (XV) and (XVI) include:

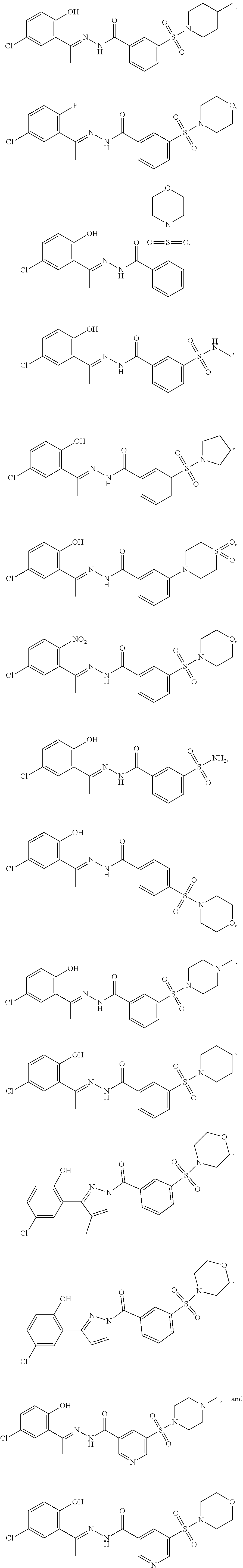

In other embodiments, LSD1 inhibitor compounds are selected from hydroxytyrosol, hydroxytyrosol derived and/or substituted compounds, and/or a hydroxytyrosol metabolites, as described for example by McCord et al. in U.S. Publication No. 2014/0155339, which is hereby incorporated by reference herein in its entirety. Representative compounds of this type include:

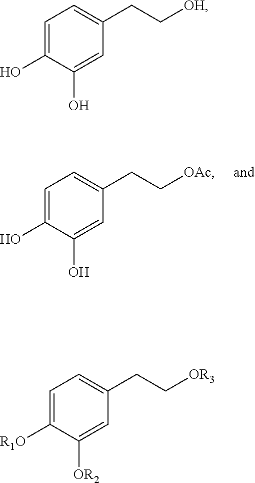

wherein: R₁, R₂ and R₃ are independently selected from the group consisting of hydrogen, substituted or unsubstituted alkyl, substituted or unsubstituted alkenyl, substituted or unsubstituted alkynyl, substituted or unsubstituted aryl, substituted or unsubstituted heterocyclyl, substituted or unsubstituted acyl, ORa, SRa, SORa, SO2Ra, OSO₂Ra, OSO₃Ra, NO₂, NHRa, N(Ra)₂, ═N—Ra, N(Ra)CORa, N(CORa)₂, N(Ra)SO2R′, N(Ra)C(═NRa)N(Ra)Ra, CN, halogen, CORa, COORa, OCORa, OCOORa, OCONHRa, OCON(Ra)₂, CONHRa, CON(Ra)₂, CON(Ra)ORa, CON(Ra)SO₂Ra, PO(ORa)₂, PO(ORa)Ra, PO(ORa)(N(Ra)Ra) and amino acid ester having inhibitory efficacy against the LSD1 protein; and further wherein each of the Ra groups is independently selected from the group consisting of hydrogen, substituted or unsubstituted alkyl, substituted or unsubstituted alkenyl, substituted or unsubstituted alkynyl, substituted or unsubstituted aryl, and substituted or unsubstituted heterocyclyl, substituted or unsubstituted acyl, and the like having inhibitory efficacy against the LSD1 protein; and further wherein each of the substituted or unsubstituted alkyl, alkenyl, alkynyl, aryl, heterocyclyl, and/or acyl groups are C₁₋₂₈ (including all ranges therein).

In still other embodiments, LSD1 inhibitor compounds are selected from small molecule compounds described by Casero et al. in U.S. Publication No. 2014/0011857, which is hereby incorporated by reference herein in its entirety. Representative compounds of this type are represented by formula (XVII):

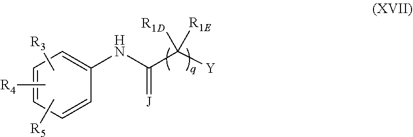

wherein:

Y is

(i)

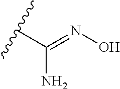 [SMILES: */C(N)=N/O]

(ii) —C(O)OH; or

(iii) —NH₂; J is O, S, or absent, wherein if J is absent, then the carbon to which J is attached is —CH₂—; R₃ is alkyl, aryl, carbocyclic, heterocyclic, aralkyl, alkoxy, aryloxy, haloalkyl, or halo, each of which is optionally substituted, nitro, hydroxy, thio, C(O)NR_{A}R_{B}, or C(O)OR_{A}; R₄ is H, alkyl, aryl, carbocyclic, heterocyclic, aralkyl, alkoxy, aryloxy, haloalkyl, or halo, each of which is optionally substituted, nitro, hydroxy, thio, C(O)NR_{A}R_{B}, or C(O)OR_{A}; R₅ is H, alkyl, aryl, carbocyclic, heterocyclic, aralkyl, alkoxy, aryloxy, haloalkyl, or halo, each of which is optionally substituted, nitro, hydroxy, thio, C(O)NR_{A}R_{B}, or C(O)OR_{A}; wherein R₃ is ortho substituted; each Rip or RAE, is independently H, alkyl, aryl, carbocyclic, heterocyclic, alkoxy, or halo, each of which is optionally substituted; RA and R_{B} are each independently selected at each occurrence from the following: [symbol] uted alkyl, optionally substituted alkenyl or optionally sub [symbol] containing 0, 1, 2, or 3 heteroatoms selected from O, S, or N; optionally substituted aryl; optionally substituted heteroaryl; optionally substituted heterocyclic; optionally substituted carbocyclic; or hydrogen; and q is 1, 2, 3, 4, 5, 6, or 7.

In other embodiments, LSD1 inhibitors are selected from arylcyclopropylamine compounds described by Munoz et al. in U.S. Publication No. 2013/0231342, which is hereby incorporated by reference herein in its entirety. Representative compounds of this type are represented by formula (XVIII):

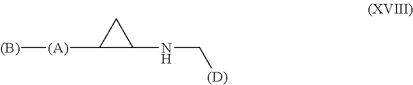

wherein:

(A) is a cyclyl group having n substituents (R3);

(B) is a cyclyl group or an -(L1)-cyclyl group, wherein said cyclyl group or the cyclyl moiety comprised in said -(L1)-cyclyl group has n substituents (R2);

(L1) is —O—, —NH—, —N(alkyl)-, alkylene or heteroalkylene;

(D) is a heteroaryl group or an -(L2)-heteroaryl group, wherein said heteroaryl group or the heteroaryl moiety comprised in said -(L2)-heteroaryl group has one substituent (R1), and further wherein said heteroaryl group is covalently bonded to the remainder of the molecule through a ring carbon atom or the heteroaryl moiety comprised in said -(L2)-heteroaryl group is covalently bonded to the (L2) moiety through a ring carbon atom;

(L2) is —O—, —NH—, —N(alkyl)-, alkylene or heteroalkylene;

(R1) is a hydrogen bonding group;

each (R2) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate or urea;

each (R3) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate, or urea; and n is independently 0, 1, 2, 3 or 4.

Non-limiting examples of compounds according to formula (XVIII) are selected from: 5-(((trans)-2-(4-(benzyloxy)phenyl)cyclopropylamino)methyl)pyrimidin-2-amine; 5-(((trans)-2-(4-(benzyloxy)phenyl)cyclopropylamino)methyl)thiazol-2-amine; 5-(((trans)-2-(6-(3-(trifluoromethyl)phenyl)pyridin-3-yl)cyclopropylamino)methyl)pyrimidin-2-amine; 5-(((trans)-2-(6-(3-(trifluoromethyl)phenyl)pyridin-3-yl)cyclopropylamino-)methyl)thiazol-2-amine; 3-(5-((trans)-2-((2-aminopyrimidin-5-yl)methylamino)cyclopropyl)pyridin-2-yl)phenol; 3-(5-((trans)-2-((2-aminothiazol-5-yl)methylamino)cyclopropyl)pyridin-2-yl)phenol; 4′-((trans)-2-((2-aminopyrimidin-5-yl)methylamino)cyclopropyl)bi-phenyl-3-ol; 4′-((trans)-2-((2-aminothiazol-5-yl)methylamino)cyclopropyl)biphenyl-3-ol; 5-(((trans)-2-(4-(benzyloxy)phenyl)cyclopropylamino)methyl)-1,2,4-oxadiazol-3-amine; 5-(((trans)-2-(4-(benzyloxy)phenyl)cyclopropylamino)methyl)-1,3,4-oxadiazol-2-amine; 5-((((trans)-2-(4-((4-fluorobenzyl)oxy)phenyl)cyclopropyl)amino)methyl)-1,3,4-oxadiazol-2-amine; 5-((((trans)-2-(4- [symbol] oxy)phenyl)cyclopropyl)amino)methyl)-1-,3,4-oxadiazol- [symbol] )-2-(4-((3,5-difluorobenzyl)oxy)phenyl)cyclopropyl)amino)methyl)-1,3,4-oxadiazol-2-amine; 5-((((trans)-2-(4-((4-chlorobenzyl)oxy)phenyl)cyclopropyl)amino)methyl)-1-,3,4-oxadiazol-2-amine; 5-((((trans)-2-(4-((3-chlorobenzyl)oxy)phenyl)cyclopropyl)amino)methyl)-1,3,4-oxadiazol-2-amine; 5-((((trans)-2-(4-((2-fluorobenzyl)oxy)phenyl)cyclopropyl)amino)methyl)-1-,3,4-oxadiazol-2-amine; 5-((((trans)-2-(4-(benzyloxy)phenyl)cyclopropyl)amino)methyl)-N-methyl-1,-3,4-oxadiazol-2-amine; N-(5-((((trans)-2-(4-(benzyloxy)phenyl)cyclopropyl)amino)methyl)-1,3,4-oxadiazol-2-yl)acetamide; 4′-((trans)-2-(((5-amino-1,3,4-oxadiazol-2-yl)methyl)amino)cyclopropyl)-[-1,1′-biphenyl]-3-ol; 5-((((trans)-2-(6-(3-(trifluoromethyl)phenyl)pyridin-3-yl)cyclopropyl)amino)methyl)-1,3,4-oxadiazol-2-amine; 5-((((trans)-2-(4-(benzyloxy)phenyl)cyclopropyl)amino)methyl)-1,3,4-thiadiazol-2-amine; 2-((((trans)-2-(4-(benzyloxy)phenyl)cyclopropyl)amino)methyl)thiazol-5-amine; 4-((((trans)-2-(3′-(trifluoromethyl)-[1,1′-biphenyl]-4-yl)cyclopropyl)amino)methyl)thiazol-2-amine; 2-((((trans)-2-(4-(benzyloxy)phenyl)cyclopropyl)amino)methyl)oxazol-5-amine; 3-((((trans)-2-(4-(benzyloxy)phenyl)cyclopropyl)amino)methyl)isoxazol-5-amine; 5-((((trans)-2-(4-(benzyloxy)phenyl)cyclopropyl)amino)methyl)-1,2,4-oxadiazol-3-amine; 3-((((trans)-2-(4-(benzyloxy)phenyl)cyclopropyl)amino)methyl)-1,2,4-oxadi-azol-5-amine; 5-((((trans)-2-(4-(benzyloxy)phenyl)cyclopropyl)amino)methyl)-1,2,4-thiad-iazol-3-amine; 5-((((trans)-2-(4-(benzyloxy)phenyl)cyclopropyl)amino)methyl)pyridin-2-amine; 6-((((trans)-2-(4-(benzyloxy)phenyl)cyclopropyl)amino)methyl)pyridazin-3-amine; 5-((((trans)-2-(4-(benzyloxy)phenyl)cyclopropyl)amino)methyl)pyrazin-2-amine; 2-((((trans)-2-(4-(benzyloxy)phenyl)cyclopropyl)amino)methyl)pyrimidin-5-amine; 6-((((trans)-2-(4-(benzyloxy)phenyl)cyclopropyl)amino)methyl)-1,2,4-triazin-3-amine; 3-((((trans)-2-(4-(benzyloxy)phenyl)cyclopropyl)amino)methyl)-1,2,4-triaz-in-6-amine; or a pharmaceutically acceptable salt or solvate thereof.

In preferred embodiments, the LSD inhibitor is an inhibitor of nuclear translocation/localization of LSD. Representative inhibitors of this type include those disclosed by Rao et al. in International Application No. PCT/AU2017/050969 filed 7 Sep. 2017, which is incorporated herein by reference in its entirety. These compounds are isolated or purified proteinaceous molecules comprising, consisting or consisting essentially of a sequence corresponding to residues 108 to 118 of LSD1.

The amino acid sequence of LSD1 (UniProt No. 060341-1) is presented in SEQ ID NO: 1. Residues 108-118 are underlined in the sequence below.

[SEQ ID NO: 1]
MLSGKKAAAAAAAAAAAATGTEAGPGTAGGSENGSEVAAQPAGLSGPAEV
GPGAVGERTPRKKEPPRASPPGGLAEPPGSAGPQAGPTVVPGSATPMETG
IAETPEGRRTSRRKRAKVEYREMDESLANLSEDEYYSEEERNAKAEKEKK
LPPPPPQAPPEEENESEPEEPSGVEGAAFQSRLPHDRMTSQEAACFPDII
SGPQQTQKVFLFIRNRTLQLWLDNPKIQLTFEATLQQLEAPYNSDTVLVH
RVHSYLERHGLINFGIYKRIKPLPTKKTGKVIIIGSGVSGLAAARQLQSF
GMDVTLLEARDRVGGRVATFRKGNYVADLGAMVVTGLGGNPMAVVSKQVN
MELAKIKQKCPLYEANGQAVPKEKDEMVEQEFNRLLEATSYLSHQLDFNV
LNNKPVSLGQALEVVIQLQEKHVKDEQIEHWKKIVKTQEELKELLNKMVN
LKEKIKELHQQYKEASEVKPPRDITAEFLVKSKHRDLTALCKEYDELAET
QGKLEEKLQELEANPPSDVYLSSRDRQILDWHFANLEFANATPLSTLSLK
HWDQDDDFEFTGSHLTVRNGYSCVPVALAEGLDIKLNTAVRQVRYTASGC
EVIAVNTRSTSQTFIYKCDAVLCTLPLGVLKQQPPAVQFVPPLPEWKTSA
VQRMGFGNLNKVVLCFDRVFWDPSVNLFGHVGSTTASRGELFLFWNLYKA
PILLALVAGEAAGIMENISDDVIVGRCLAILKGIFGSSAVPQPKETVVSR
WRADPWARGSYSYVAAGSSGNDYDLMAQPITPGPSIPGAPQPIPRLFFAG
EHTIRNYPATVHGALLSGLREAGRIADQFLGAMYTLPRQATPGVPAQQSP
SM.

[symbol]

n some embodiments, the proteinaceous molecule is an is [symbol] proteinaceous molecule represented by formula XIX:

Z₁RRTX₁RRKRAKVZ₂ (XIX)

wherein:

“Z₁” and “Z₂” are independently absent or are independently selected from at least one of a proteinaceous moiety comprising from about 1 to about 50 amino acid residues (and all integer residues in between), and a protecting moiety; and

“X₁” is selected from small amino acid residues, including S, T, A, G and modified forms thereof.

In some embodiments, “X₁” is selected from S and A.

In some embodiments, “X₁” is selected from S, A and modified forms thereof. In some embodiments, “X₁” is selected from S, A and S(PO₃).

In some embodiments, “X₁” is a modified form of S, especially S(PO₃).

In some embodiments, “Z₁” is a proteinaceous molecule represented by Formula XX:

X₂X₃X₄ (XX)

wherein:

“X₂” is absent or is a protecting moiety;

“X₃” is absent or is selected from any amino acid residue; and

“X₄” is selected from any amino acid residue.

In some embodiments, “X₃” is selected from basic amino acid residues including R, K and modified forms thereof. In some embodiments, “X₃” is R.

In some embodiments, “X₄” is selected from aromatic amino acid residues, including F, Y, W and modified forms thereof. In some embodiments, “X₄” is W.

In some embodiments, “Z₂” is absent.

In some embodiments, the isolated or purified proteinaceous molecule of Formula XIX comprises, consists or consists essentially of an amino acid sequence represented by SEQ ID NO: 2, 3 or 4:

[SEQ ID NO: 2]
RRTSRRKRAKV;
[SEQ ID NO: 3]
RRTARRKRAKV;
or
[SEQ ID NO: 4]
RWRRTARRKRAKV.

In particular embodiments, the isolated or purified proteinaceous molecule of formula XIX comprises, consists or consists essentially of an amino acid sequence represented by SEQ ID NO: 2 or 3.

In some embodiments, the isolated or purified proteinaceous molecule of formula XIX comprises, consists or consists essentially of the amino acid sequence of SEQ ID NO: 5:

[SEQ ID NO: 5]
GRRTSRRKRAKVE.

In some embodiments, the isolated or purified proteinaceous molecule of formula XIX is other than a proteinaceous molecule consisting of the amino acid sequence of SEQ ID NO: 5.

In some embodiments of the proteinaceous molecules according to formula (XIX), the molecules comprise at least one membrane permeating moiety. The membrane permeating moiety may be conjugated at any point of the proteinaceous molecule. Suitable membrane permeating moieties include lipid moieties, cholesterol and proteins, such as cell-penetrating peptides and polycationic peptides; especially lipid moieties.

Non-limiting examples of cell-penetrating peptides include the peptides described in, for example, US 20090047272, US 20150266935 and US 20130136742. Accordingly, suitable cell penetrating peptides may include, but are not limited to, basic poly(Arg) and poly(Lys) peptides and basic poly(Arg) and poly(Lys) peptides containing non-natural analogues of Arg and Lys residues such as YGRKKRPQRRR [SEQ ID NO:6] (HIV TAT₄₇₋₅₇), RRWRRWWRRWWRRWRR (W/R) [SEQ ID NO:7], CWK₁₈(AlkCWK₁₈) [SEQ ID NO:8], K₁₈WCCWK₁₈(Di-CWK₁₈) [SEQ ID NO:9], WTLNSAGYLLGKINLKALAALAKKIL [SEQ ID NO:10] (Transportan), GLFEALEELWEAK [SEQ ID NO:11] (DipaLytic), K₁₆GGCRGDMFGCAKi₆RGD(K₁₆RGD) [SEQ ID NO:12], K₁₆GGCMFGCGG [SEQ ID NO:13] (P1), K₁₆ICRRARGDNPDDRCT [SEQ ID NO:14] (P2), KKWKMRRNQFWVKVQRbAK(B)bA [SEQ ID NO:15] (P3), VAYISRGGVSTYYSDTVKGRFTRQKYNKRA [SEQ ID NO:16] (P3a), IGRIDPANGKTKYAPKFQDKATRSNYYGNSPS [SEQ ID NO:17] (P9.3), KETWWETWWTEWSQPKKKRKV [SEQ ID NO:18] (Pep-1), PLAEIDGIELTY [SEQ ID NO:19] (Plae), K₁₆GGPLAEIDGIELGA [SEQ ID NO:20] (Kplae), K₁₆GGPLAEIDGIELCA [SEQ ID NO:21] (cKplae), GALFLGFLGGAAGSTMGAWSQPKSKRKV [SEQ ID NO:22] (MGP), WEAK(LAKA)₂-LAKH(LAKA)₂LKAC [SEQ ID NO:23] (HA2), (LARL)₆NHCH₃ (LARL)₆ [SEQ ID NO:24], KLLKLLLKLWLLKLLL [SEQ ID NO:25] (Hel-11-7), (KKKK)₂GGC (KK) [SEQ ID NO:26], (KWKK)₂GCC (KWK) [SEQ ID NO:27], (RWRR)₂GGC (RWR) [SEQ ID NO:28], PKKKRKV (SV40 NLS7), PEVKKKRKPEYP [SEQ ID NO:29] (NLS12), TPPKKKRKVEDP [SEQ ID NO:30] (NLS12a), GGGGPKKKRKVGG [SEQ ID NO:31] (SV40 NLS13), GGGFSTSLRARKA [SEQ ID NO:32] (AV NLS13), CKKKKKKSEDEYPYVPN [SEQ ID NO:33] (AV RME NLS17), CKKKKKKKSEDEYPYVPNFSTSLRARKA [SEQ ID NO:34] (AV FP NLS28), LVRKKRKTEEESPLKDKDAKKSKQE [SEQ ID NO:35] (SV40 N1 NLS24), and K₉K₂K₄K₈GGK₅ (Loligomer); HSV-1 tegument protein VP22; HSV-1 tegument protein VP22r fused with nuclear export signal (NES); mutant B-subunit of Escherichia coli enterotoxin EtxB (H57S); detoxified exotoxin A (ETA); the protein transduction domain of the HIV-1 Tat protein, GRKKRRQRRRPPQ [SEQ ID NO:36]; the Drosophila melanogaster Antennapedia domain Antp (amino acids 43-58), RQIKIWFQNRRMKWKK [SEQ ID NO:37]; Buforin II, TRSSRAGLQFPVGRVHRLLRK [SEQ ID NO:38]; hClock-(amino acids 35-47) (human Clock protein DNA-binding peptide), KRVSRNKSEKKRR [SEQ ID NO:39]; MAP (model amphipathic peptide), KLALKLALKALKAALKLA [SEQ ID NO:40]; K-FGF, AAVALLPAVLLALLAP [SEQ ID NO:41]; Ku70-derived peptide, comprising a peptide selected from the group comprising VPMLKE, VPMLK, PMLKE or PMLK; Prion, Mouse Prpe (amino acids 1-28), MANLGYWLLALFVTMWTDVGLCKKRPKP [SEQ ID NO:42]; pVEC, LLIILRRRIRKQAHAHSK [SEQ ID NO:43]; Pep-I, KETWWETWWTEWSQPKKKRKV [SEQ ID NO:44]; SynBI, RGGRLSYSRRRFSTSTGR [SEQ ID NO:45]; Transportan, GWTLNSAGYLLGKINLKALAALAKKIL [SEQ ID NO:46]; Transportan-10, AGYLLGKINLKALAALAKKIL [SEQ ID NO:47]; CADY, Ac-GLWRALWRLLRSLWRLLWRA-cysteamide [SEQ ID NO:48]; Pep-7, SDLWEMMMVSLACQY [SEQ ID NO:49]; HN-1, TSPLNIHNGQKL [SEQ ID NO:50]; VT5, DPKGDPKGVTVTVTVTVTGKGDPKPD [SEQ ID NO:51]; or pISL, RVIRVWFQNKRCKDKK [SEQ ID NO:52]. [symbol] preferred embodiments, the membrane permeating mo [symbol] such as a C₁₀-C₂₀ fatty acyl group, especially octadecanoyl (stearoyl; C₁₈), hexadecanoyl (palmitoyl; C₁₆) or tetradecanoyl (myristoyl; C₁₄); most especially tetradecanoyl. In preferred embodiments, the membrane permeable moiety is conjugated to the N- or C-terminal amino acid residue or through the amine of a lysine side-chain of the proteinaceous molecule, especially the N-terminal amino acid residue of the proteinaceous moiety.

2.2 PD-1 Binding Antagonists

PD-1 binding antagonists are suitably molecules that inhibit signaling through PD-1 and include molecules that inhibit the binding of PD-1 to its ligand binding partners. In some embodiments, the PD-1 ligand binding partners are PD-L1 and/or PD-L2. The antagonist may be an antibody, an immunoadhesin, a fusion protein, or oligopeptide.

The PD-1 binding antagonist is preferably an anti-PD-1 antibody (e.g., a human antibody, a humanized antibody, or a chimeric antibody). In some embodiments, the anti-PD-1 antibody is selected from the group consisting of MDX-1106 (nivolumab, OPDIVO), Merck 3475 (MK-3475, pembrolizumab, KEYTRUDA), CT-011 (pidilizumab), MEDI-4736 (durvalumab) MEDI-0680 (AMP-514), PDR001, REGN2810, BGB-108, and BGB-A317. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT, hBAT-1 or Pidilizumab, is an anti-PD-1 antibody described in WO2009/101611. AMP-224, also known as B7-DCIg, is a PD-L2-Fc fusion soluble receptor described in WO2010/027827 and WO2011/066342.

In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO:53 and/or a light chain variable region comprising the light chain variable region amino acid sequence from SEQ ID NO:54. In a still further embodiment, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

[SEQ ID NO: 53]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,

or (b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

[SEQ ID NO: 54] [symbol]
IVLTQSPATLSLSPGERATLSCRASQSVSSYLAWYQQKPGQAP [symbol] RFSG
SGSGTDFTLTISSLEPEDFAVYYCQQSSNWPRTFGQGTKVEIKRTNVAAP
SVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGE
C. [symbol]
indicates data missing or illegible when filed

In some embodiments, the anti-PD-1 antibody is pembrolizumab (CAS Registry Number: 1374853-91-4). In a still further embodiment, provided is an isolated anti-PD-1 antibody comprising a heavy chain variable region comprising the heavy chain variable region amino acid sequence from SEQ ID NO: 55 and/or a light chain variable region comprising the light chain variable region amino acid sequence from SEQ ID NO: 56. In a still further embodiment, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence:

[SEQ ID NO: 55]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK,

or (b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

[SEQ ID NO: 56]
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

The present invention also contemplates antibody fragments comprising heavy and light chain HVRs of a full-length anti-PD-1 antagonist antibody.

In a still further aspect, provided herein are nucleic acids encoding any of the antibodies described herein. In some embodiments, the nucleic acid further comprises a vector suitable for expression of the nucleic acid encoding any of the previously described anti-PDL1, anti-PD-1, or anti-PDL2 antibodies. In a still further specific aspect, the vector further comprises a host cell suitable for expression of the nucleic acid. In a still further specific aspect, the host cell is a eukaryotic cell or a prokaryotic cell. In a still further specific aspect, the eukaryotic cell is a mammalian cell, such as Chinese Hamster Ovary (CHO).

The antibody or antigen binding fragment thereof, may be made using methods known in the art, for example, by a process comprising culturing a host cell containing nucleic acid encoding any of the previously described anti-PD-1, or antigen-binding fragment in a form suitable for expression, under conditions suitable to produce such antibody or fragment, and recovering the antibody or fragment. [symbol] some embodiments, the isolated anti-PD-1 antibody is

[symbol]

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

2.3 Ancillary Agents

In some embodiments, the LSD inhibitor and PD-1 binding antagonist are administered concurrently with an ancillary agent for treating, or for aiding in the treatment of, a T-cell dysfunctional disorder. Non-limiting examples of ancillary agents include cytotoxic agents, gene therapy agents, DNA therapy agents, viral therapy agents, RNA therapy agents, immunotherapeutic agents, bone marrow transplantation agents, nanotherapy agents, or a combination of the foregoing. The ancillary agent may be in the form of adjuvant or neoadjuvant therapy. In some embodiments, the ancillary agent is a small molecule enzymatic inhibitor or anti-metastatic agent. In some embodiments, the ancillary agent is a side-effect limiting agent (e.g., agents intended to lessen the occurrence and/or severity of side effects of treatment, such as anti-nausea agents, etc.). In some embodiments, the ancillary agent is a radiotherapy agent. In some embodiments, the ancillary agent is an agent that targets PI3K/AKT/nnTOR pathway, HSP90 inhibitor, tubulin inhibitor, apoptosis inhibitor, and/or chemopreventative agent. In some embodiments, the ancillary agent is an immunotherapeutic, e.g., a blocking antibody, ipilimumab (also known as MDX-010, MDX-101, or Yervoy®), tremelimumab (also known as ticilimumab or CP-675,206), an antagonist directed against B7-H3 (also known as CD276), e.g., a blocking antibody, MGA271, an antagonist directed against a TGF-β, e.g., metelimumab (also known as CAT-192), fresolimumab (also known as GC1008), or LY2157299, a T cell (e.g., a cytotoxic T cell or CTL) expressing a chimeric antigen receptor (CAR), a T cell comprising a dominant-negative TGF-β receptor, e.g., a dominant-negative TGF-β type II receptor, an agonist directed against CD137 (also known as TNFRSF9, 4-1BB, or ILA), e.g., an activating antibody, urelumab (also known as BMS-663513), an agonist directed against CD40, e.g., an activating antibody, CP-870893, an agonist directed against OX40 (also known as CD134), e.g., an activating antibody, administered in conjunction with an anti-OX40 antibody (e.g., AgonOX), an agonist directed against CD27, e.g., an activating antibody, CDX-1127, indoleamine-2,3-dioxygenase (IDO), 1-methyl-D-tryptophan (also known as 1-D-MT), an antibody-drug conjugate (in some embodiments, comprising mertansine or monomethyl auristatin E (MMAE)), an anti-NaPi2b antibody-MMAE conjugate (also known as DNIB0600A or RG7599), trastuzumab emtansine (also known as T-DM1, ado-trastuzumab emtansine, or KADCYLA®, Genentech), DMUC5754A, an antibody-drug conjugate targeting the endothelin B receptor (EDNBR), e.g., an antibody directed against EDNBR conjugated with MMAE, an angiogenesis inhibitor, an antibody directed against a VEGF, e.g., VEGF-A, [symbol] o known as AVASTIN®, Genentech), an antibody directe [symbol] n 2 (also known as Ang2), MEDI3617, an antineoplastic agent, an agent targeting CSF-1R (also known as M-CSFR or CD115), anti-CSF-1R (also known as IMC-CS4), an interferon, for example IFN-α or IFN-γ, Roferon-A, GM-CSF (also known as recombinant human granulocyte macrophage colony stimulating factor, rhu GM-CSF, sargramostim, or Leukine®), IL-2 (also known as aldesleukin or Proleukin®), IL-12, an antibody targeting CD20 (in some embodiments, the antibody targeting CD20 is obinutuzumab (also known as GA101 or Gazyva®) or rituximab), an antibody targeting GITR (in some embodiments, the antibody targeting GITR is TRX518), in conjunction with a cancer vaccine (in some embodiments, the cancer vaccine is a peptide cancer vaccine, which in some embodiments is a personalized peptide vaccine; in some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21, 2013)), in conjunction with an adjuvant, a TLR agonist, e.g., Poly-ICLC (also known as Hiltonol®), LPS, MPL, or CpG ODN, TNF-α, IL-1, HMGB1, an IL-10 antagonist, an IL-4 antagonist, an IL-13 antagonist, an HVEM antagonist, an ICOS agonist, e.g., by administration of ICOS-L, or an agonistic antibody directed against ICOS, an agent targeting CX3CL1, an agent targeting CXCL10, an agent targeting CCLS, an LFA-1 or ICAM1 agonist, a Selectin agonist, a targeted therapeutic agent, an inhibitor of B-Raf, vemurafenib (also known as Zelboraf®, dabrafenib (also known as Tafinlar®), erlotinib (also known as Tarceva®), an inhibitor of a MEK, such as MEK1 (also known as MAP2K1) or MEK2 (also known as MAP2K2). cobimetinib (also known as GDC-0973 or XL-518), trametinib (also known as Mekinist®), an inhibitor of K-Ras, an inhibitor of c-Met, onartuzumab (also known as MetMAb), an inhibitor of Alk, AF802 (also known as CH5424802 or alectinib), an inhibitor of a phosphatidylinositol 3-kinase (PI3K), BKM120, idelalisib (also known as GS-1101 or CAL-101), perifosine (also known as KRX-0401), an Akt, MK2206, GSK690693, GDC-0941, an inhibitor of mTOR, sirolimus (also known as rapamycin), temsirolimus (also known as CCI-779 or Torisel®), everolimus (also known as RAD001), ridaforolimus (also known as AP-23573, MK-8669, or deforolimus), OSI-027, AZD8055, INK128, a dual PI3K/mTOR inhibitor, XL765, GDC-0980, BEZ235 (also known as NVP-BEZ235), BGT226, GSK2126458, PF-04691502, PF-05212384 (also known as PKI-587). The ancillary agent may be one or more of the cytotoxic or chemotherapeutic agents described herein.

In some embodiments, the ancillary agent is an anti-infective drug. The anti-infective drugs is suitably selected from antimicrobials, which include without limitation compounds that kill or inhibit the growth of microorganisms such as viruses, bacteria, yeast, fungi, protozoa, etc. and thus include antibiotics, amebicides, antifungals, antiprotozoals, antimalarials, antituberculotics and antivirals. Anti-infective drugs also include within their scope anthelmintics and nematocides. Illustrative antibiotics include quinolones (e.g., amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin; gemifloxacin; and garenoxacin), tetracyclines, glycylcyclines and oxazolidinones (e.g., chlortetracycline, demeclocycline, doxycycline, lymecycline, methacycline, minocycline, oxytetracycline, tetracycline, tigecycline; linezolide, eperozolid), glycopeptides, aminoglycosides (e.g., amikacin, arbekacin, butirosin, dibekacin, fortimicins, gentamicin, kanamycin, meomycin, netilmicin, ribostamycin, sisomicin, spectinomycin, streptomycin, tobramycin), β-lactams (e.g., imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpinnizole, cefpiramide, [symbol] sulodin, ceftazidime, cefteram, ceftezole, ceftibuten, cefti [symbol] cefuroxime, cefuzonam, cephaacetrile, cephalexin, cephaloglycin, cephaloridine, cephalothin, cephapirin, cephradine, cefinetazole, cefoxitin, cefotetan, azthreonam, carumonam, flomoxef, moxalactam, amidinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g., azithromycin, clarithromycin, erythromycin, oleandomycin, rokitamycin, rosaramicin, roxithromycin, troleandomycin), ketolides (e.g., telithromycin, cethromycin), coumermycins, lincosamides (e.g., clindamycin, lincomycin) and chloramphenicol. Illustrative antivirals include abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivudine/zidovudine, nelfinavir mesylate, nevirapine, oseltamivir phosphate, ribavirin, rimantadine hydrochloride, ritonavir, saquinavir, saquinavir mesylate, stavudine, valacyclovir hydrochloride, zalcitabine, zanamivir, and zidovudine. Non-limiting examples of amebicides or antiprotozoals include atovaquone, chloroquine hydrochloride, chloroquine phosphate, metronidazole, metronidazole hydrochloride, and pentamidine isethionate. Anthelmintics can be at least one selected from mebendazole, pyrantel pamoate, albendazole, ivermectin and thiabendazole. Illustrative antifungals can be selected from amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin, and terbinafine hydrochloride. Non-limiting examples of antimalarials include chloroquine hydrochloride, chloroquine phosphate, doxycycline, hydroxychloroquine sulfate, mefloquine hydrochloride, primaquine phosphate, pyrimethamine, and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine, and streptomycin sulfate.

### 3. Pharmaceutical Compositions and Formulations

Also provided herein are pharmaceutical compositions and formulations comprising a LSD inhibitor (e.g., a LSD1 inhibitor, a nuclear LSD inhibitor, etc.), a PD-1 binding antagonist and a pharmaceutically acceptable carrier. In some embodiments, the pharmaceutical compositions and formulations further comprise an ancillary agent as described for example herein. In specific examples of this type, the ancillary agent is one that targets rapidly dividing cells and/or disrupt the cell cycle or cell division (e.g., a cytotoxic compound such as a taxane).

Pharmaceutical compositions and formulations as described herein can be prepared by mixing the active ingredients (e.g., a small molecule, nucleic acid, or polypeptide) having the desired degree of purity with one or more optional pharmaceutically acceptable carriers (Remington's Pharmaceutical Sciences 16th edition, Osol, A. Ed. (1980)). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or [symbol] ; hydrophilic polymers such as polyvinylpyrrolidone; ami [symbol] ine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, especially relating to peptide and polypeptide active agents (e.g., antibodies, inhibitory peptides and immunoadhesins), the active agents and optional pharmaceutically acceptable carriers are in the form of lyophilized formulations or aqueous solutions. Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The compositions and formulations herein may also contain further active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (e.g., liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility may be readily accomplished, e.g., by filtration through sterile filtration membranes.

Depending on the specific conditions being treated, the formulations may be administered systemically or locally. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Techniques for formulation and administration may be found in “Remington's Pharmaceutical Sciences,” Mack Publishing Co., Easton, Pa., latest edition.

### 4. Therapeutic Uses

The present invention discloses that a LSD inhibitor (e.g., a LSD1 inhibitor, a nuclear LSD inhibitor, etc.) and a PD-1 binding antagonist (also referred to herein as the “dual therapy”) are useful for treating a T-cell dysfunctional disorder, or for enhancing immune function (e.g., immune effector function, T-cell function etc.) in an individual having cancer, for treating or [symbol] gression of cancer, or for treating infection in an individua [symbol] embodiments, the therapeutic combination is disclosed for treating or delaying the progression of cancer, including metastatic cancer, and for preventing cancer recurrence. Any of the LSD inhibitors and PD-1 binding antagonists known in the art or described herein may be used in this regard.

In specific embodiments, the combination therapy further comprises the use or administration of an ancillary agent (e.g., a chemotherapeutic agent), as described for example herein. In advantageous example of this type, the ancillary agent is one that targets rapidly dividing cells and/or disrupt the cell cycle or cell division (e.g., a cytotoxic compound such as a taxane). In these embodiments, the combination therapy is referred to herein as “triple therapy”).

Suitably, the individual to be treated with the combination therapy comprises a T-cell (e.g., a CD8⁺ T-cell or CD4⁺ T-cell) with a mesenchymal phenotype, for example, a T-cell that expresses nuclear LSD at a higher level than the level of expression of TBET in the same T-cell, and/or at a higher level than in an activated T-cell. The T-cell may be a tumor-infiltrating lymphocyte or a circulating lymphocyte. The T-cell suitably exhibits T-cell exhaustion or anergy and in representative examples of this type, the T-cell expresses a higher level of EOMES than TBET. In some embodiments, the T-cell has impaired or repressed immune function and suitably expresses biomarkers of reduced T-cell activation (e.g., reduced production and/or secretion of cytokines such as IL-2, IFN-γ and TNF-α). In these embodiments, the T-cell suitably expresses EOMES in the nucleus of the T cell at a higher level than the level of TBET in the same T-cell or the level of EOMES in the nucleus of an activated T-cell. Accordingly, nuclear LSD, EOMES and TBET, together with PD-1, which is known marker of T-cell exhaustion, (also referred to herein as “T-cell function biomarkers”) can be used to determine the immune function of T cells in a patient for assessing a patient's T-cell immune status, including susceptibility to treatment with PD-1 binding antagonists.

In some embodiments, the individual is a human.

In some embodiments, the individual has been treated with a PD-1 binding antagonist before the combination treatment with a PD-1 binding antagonist and a LSD inhibitor (e.g., a nuclear translocation inhibitor of LSD).

In some embodiments, the individual has cancer that is resistant (has been demonstrated to be resistant) to one or more PD-1 binding antagonists. In some embodiments, resistance to a PD-1 antagonist includes recurrence of cancer or refractory cancer. Recurrence may refer to the reappearance of cancer, in the original site or a new site, after treatment. In some embodiments, resistance to a PD-1 binding antagonist includes progression of the cancer during treatment with the PD-1 binding antagonist. In some embodiments, resistance to a PD-1 binding antagonist includes cancer that does not respond to treatment. The cancer may be resistant at the beginning of treatment or it may become resistant during treatment. In some embodiments, the cancer is at early stage or at late stage.

In some embodiments of any of the methods, assays and/or kits, any one or more of the T-cell function biomarkers are detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof. [symbol] some embodiments of any of the methods, assays and/ [symbol] ore of the T-cell function biomarkers are detected in the sample by protein expression. In some embodiments, protein expression is determined by immunohistochemistry (IHC). In some embodiments, any one or more of the T-cell function biomarkers are detected using an antibody that binds specifically to a respective biomarker. In some embodiments, nuclear LSD and/or EOMES biomarkers are detected in the nucleus of a T-cell, for example using IHC. In some embodiments, a complex comprising nuclear LSD and EOMES biomarkers is detected in the nucleus of a T-cell.

In some embodiments, the combination therapy of the invention comprises administration of a LSD inhibitor and a PD-1 binding antagonist. The LSD inhibitor and PD-1 binding antagonist may be administered in any suitable manner known in the art. For example, The LSD inhibitor and PD-1 binding antagonist is typically administered concurrently. In some embodiments, the LSD inhibitor is in a separate composition as the PD-1 binding antagonist. In some embodiments, the LSD inhibitor is in the same composition as the PD-1 binding antagonist. Accordingly, the combination therapy may involve administering the LSD inhibitor separately, simultaneously or sequentially with PD-1 binding antagonist. In some embodiments, this may be achieved by administering a single composition or pharmacological formulation that includes both types of agent, or by administering two separate compositions or formulations at the same time, wherein one composition includes the LSD inhibitor and the other, PD-1 binding antagonist. In other embodiments, the treatment with the LSD inhibitor may precede or follow the treatment with the PD-1 binding antagonist by intervals ranging from minutes to days. In embodiments where the LSD inhibitor is applied separately to the PD-1 binding antagonist, one would generally ensure that a significant period of time did not expire between the time of each delivery, such that the LSD inhibitor would still be able to exert an advantageously effect on a functionally repressed T-cell (e.g., a mesenchymal T-cell) as noted above, and in particular, to render the T-cell with enhanced immune function, including susceptibility of the T-cell to reinvigoration by the PD-1 binding antagonist. In such instances, it is contemplated that one would administer both modalities within about 1-12 hours of each other and, more suitably, within about 2-6 hours of each other. In some situations, it may be desirable to extend the time period for treatment significantly, however, where several hours (2, 3, 4, 5, 6 or 7) to several days (1, 2, 3, 4, 5, 6, 7 or 8) lapse between the respective administrations.

It is conceivable that more than one administration of either the LSD inhibitor or the PD-1 binding antagonist will be desired. Various combinations may be employed, where the LSD inhibitor is “A” and the PD-1 binding antagonist is “B”, as exemplified below:

A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B.

In some embodiments, the combination therapy of the invention comprises administration of a LSD inhibitor, a PD-1 binding antagonist and a chemotherapeutic agent. These agents may be administered in any suitable manner known in the art. For example, The LSD inhibitor, PD-1 binding antagonist and chemotherapeutic agent may be administered concurrently. In some embodiments, the LSD inhibitor, PD-1 binding antagonist and chemotherapeutic agent are in separate compositions. In other embodiments, the LSD inhibitor, PD-1 binding antagonist and chemotherapeutic agent are in the same composition. In still other embodiments, the LSD inhibitor is in the same composition as the PD-1 binding antagonist and the chemotherapeutic agent is in a [symbol] ition. In further embodiments, the LSD inhibitor is in the [symbol] the chemotherapeutic agent and the PD-1 binding antagonist is in a separate composition. Accordingly, the combination therapy may involve administering the LSD inhibitor separately, simultaneously or sequentially with PD-1 binding antagonist and chemotherapeutic agent. In some embodiments, this may be achieved by administering a single composition or pharmacological formulation that includes the three types of agent, or by administering separate compositions or formulations at the same time. In other embodiments, the treatment with one agent may precede or follow the treatment with the other two agents by intervals ranging from minutes to days. In embodiments where one agent is applied separately to the other agent, one would generally ensure that a significant period of time did not expire between the time of each delivery, such that the LSD inhibitor would still be able to exert an advantageously effect on a functionally repressed T-cell (e.g., a mesenchymal T-cell) as noted above, and in particular, to render the T-cell with enhanced immune function, including susceptibility of the T-cell to reinvigoration by the PD-1 binding antagonist. In such instances, it is contemplated that one would administer the different modalities within about 1-12 hours of each other and, more suitably, within about 2-6 hours of each other. In some situations, it may be desirable to extend the time period for treatment significantly, however, where several hours (2, 3, 4, 5, 6 or 7) to several days (1, 2, 3, 4, 5, 6, 7 or 8) lapse between the respective administrations.

The LSD inhibitor and PD-1 binding antagonist and optionally the chemotherapeutic agent may be administered by the same route of administration or by different routes of administration. In some embodiments, the PD-1 binding antagonist is administered intravenously, intramuscularly, subcutaneously, topically, orally, transdermally, intraperitoneally, intraorbitally, by implantation, by inhalation, intrathecally, intraventricularly, or intranasally. In some embodiments, the LSD inhibitor is administered intravenously, intramuscularly, subcutaneously, topically, orally, transdermally, intraperitoneally, intraorbitally, by implantation, by inhalation, intrathecally, intraventricularly, or intranasally. In some embodiments, the chemotherapeutic agent is administered intravenously, intramuscularly, subcutaneously, topically, orally, transdermally, intraperitoneally, intraorbitally, by implantation, by inhalation, intrathecally, intraventricularly, or intranasally. An effective amount of the LSD inhibitor, PD-1 binding antagonist and optionally the chemotherapeutic agent may be administered for prevention or treatment of disease. The appropriate dosage of the LSD inhibitor, PD-1 binding antagonist and optionally the chemotherapeutic agent may be determined based on the type of disease to be treated, the type of the LSD inhibitor, PD-1 binding antagonist and optionally the chemotherapeutic agent, the severity and course of the disease, the clinical condition of the individual, the individual's clinical history and response to the treatment, and the discretion of the attending physician. In some embodiments, combination treatment with LSD inhibitor (e.g., an enzymatic or nuclear translocation inhibitor of LSD), PD-1 binding antagonists (e.g., anti-PD-1 antibody) and optionally the chemotherapeutic agent are synergistic, whereby an efficacious dose of a PD-1 binding antagonists (e.g., anti-PD-1 antibody) and/or chemotherapeutic agent in the combination is reduced relative to efficacious dose of the PD-1 binding antagonists (e.g., anti-PD-1 antibody) and/or chemotherapeutic agent as a single agent.

As a general proposition, the therapeutically effective amount of a peptide or polypeptide active agent (e.g., an antibody, peptide inhibitor, immunoadhesin, etc.) administered to a human will be in the range of about 0.01 to about 50 mg/kg of patient body weight whether by one or more administrations. In some embodiments, the antibody used is about 0.01 to about [symbol] 0.01 to about 40 mg/kg, about 0.01 to about 35 mg/kg, [symbol] 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the peptide or polypeptide active agent (e.g., an antibody, peptide inhibitor, immunoadhesin, etc.) is administered at 15 mg/kg. However, other dosage regimens may be useful. In one embodiment, an anti-PDL1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The dose of peptide or polypeptide active agent (e.g., an antibody, peptide inhibitor, immunoadhesin, etc.) administered in a combination treatment may be reduced as compared to a single treatment. The progress of this therapy is easily monitored by conventional techniques.

Small molecule compounds are generally administered at an initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed.

In any event, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch or pump. In some embodiments, the LSD inhibitor, PD-1 binding antagonist and optionally an ancillary agent (e.g., a chemotherapeutic agent) are administered on a routine schedule. Alternatively, the combination therapy may be administered as symptoms arise.

A “routine schedule” as used herein, refers to a predetermined designated period of time. The routine schedule may encompass periods of time which are identical or which differ in length, as long as the schedule is predetermined. For instance, the routine schedule may involve administration of the LSD inhibitor, PD-1 binding antagonist and optional chemotherapeutic agent on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve concurrent administration of the LSD inhibitor, PD-1 binding [symbol] ptional chemotherapeutic agent on a daily basis for the fi [symbol] / a monthly basis for several months, and then every three months after that. Any particular combination would be covered by the routine schedule as long as it is determined ahead of time that the appropriate schedule involves administration on a certain day.

In some embodiments, the treatment methods and uses may further comprise an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. In some embodiments, the additional therapy is radiation therapy. In some embodiments, the additional therapy is surgery. In some embodiments, the additional therapy is a combination of radiation therapy and surgery. In some embodiments, the additional therapy is gamma irradiation.

The efficacy of any of the methods described herein (e.g., combination treatments including administering an effective amount of a combination of LSD inhibitor, PD-1 binding antagonist and optional chemotherapeutic agent may be tested in various models known in the art, such as clinical or pre-clinical models. Suitable pre-clinical models are exemplified herein and further may include without limitation ID8 ovarian cancer, GEM models, B16 melanoma, RENCA renal cell cancer, CT26 colorectal cancer, MC38 colorectal cancer, and Cloudman melanoma models of cancer.

The efficacy of any of the methods described herein (e.g., combination treatments including administering an effective amount of a combination of LSD inhibitor, PD-1 binding antagonist and optional chemotherapeutic agent) may be tested in a GEM model that develops tumors, including without limitation GEM models of non-small-cell lung cancer, pancreatic ductal adenocarcinoma, or melanoma. For example, a mouse expressing Kras^{G12D} in a p53^{null} background after adenoviral recombinase treatment as described in Jackson et al. (2001 Genes Dev. 15(24):3243-8) (description of Kras^{G12D}) and Lee et al. (2012 Dis. Model Mech. 5(3):397-402) (FRT-mediated p53^{null} allele) may be used as a pre-clinical model for non-small-cell lung cancer. As another example, a mouse expressing Kras^{G12D} in a p16/p19^{null} background as described in Jackson et al. (2001, supra) (description of Kras^{G12D}) and Aguirre et al. (2003 Genes Dev. 17(24):3112-26) (p16/p19^{null} allele) may be used as a pre-clinical model for pancreatic ductal adenocarcinoma (PDAC). As a further example, a mouse with melanocytes expressing Braf^{V600E} in a melanocyte-specific PTEN^{null} background after inducible (e.g., 4-OHT treatment) recombinase treatment as described in Dankort et al. (2007 Genes Dev. 21(4):379-84) (description of Braf.sup.V600E) and Trotman et al. (2003 PLoS Biol. 1(3):E59) (PTEN^{null} allele) may be used as a pre-clinical model for melanoma. For any of these exemplary models, after developing tumors, mice are randomly recruited into treatment groups receiving combination LSD inhibitor, PD-1 binding antagonist and optional chemotherapeutic agent treatment or control treatment. Tumor size (e.g., tumor volume) is measured during the course of treatment, and overall survival rate is also monitored.

In some embodiments of the methods of the present disclosure, the cancer (in some embodiments, a sample of the patient's cancer as examined using a diagnostic test, as described for example herein) comprises tumor-infiltrating lymphocytes (TILs), wherein the TILs are within or otherwise associated with the cancer tissue. In these embodiments, the TILs are assessed for expression of any one or more of the T-cell function biomarkers disclosed herein. For example, nuclear LSD and EOMES can be used as biomarkers of mesenchymal phenotype and T- [symbol] addition, TBET and/or PD-1 can be used as biomarkers [symbol] which is characterized for example by high levels of inhibitory co-receptors and lacking the capacity to produce effector cytokines (Wherry, E. J. 2011 Nature immunology 12: 492-499; Rabinovich et al., 2007 Annual Review of immunology 25:267-296).

In some embodiments of the methods of the present disclosure, the individual has a T-cell dysfunction that manifests in a T-cell dysfunctional disorder. The T-cell dysfunctional disorder may be characterized by T-cell anergy or decreased ability to secrete cytokines, proliferate or execute cytolytic activity. In some embodiments of the methods of the present disclosure, the T-cell dysfunctional disorder is characterized by repressed T-cell immune function. In some embodiments of the methods of the present disclosure, the T-cell dysfunctional disorder is characterized by T-cell of a mesenchymal phenotype. In some embodiments of the methods of the present disclosure, the T-cell dysfunctional disorder is characterized by T-cell exhaustion. In some embodiments of the methods of the present disclosure, the T-cells are CD4⁺ and/or CD8⁺ T cells. In accordance with the present invention, LSD inhibitor treatment may increase expression of biomarkers of T-cell activation and effector capacity (e.g., IFN-γ, TNF-α, Ki67 and TBET), decrease expression of biomarkers of T-cell exhaustion (e.g., EOMES), and increase activation and proliferation of T-cells, including effector and memory T-cells. Notably, LSD inhibitor treatment may confer enhanced susceptibility of exhausted T-cells to reinvigoration by PD-1 binding antagonists. As such, the combination treatment LSD inhibitor and a PD-1 binding antagonist may increase T-cell (e.g., CD4⁺ T-cell, CD8⁺ T-cell, memory T-cell) priming, activation and/or proliferation relative to prior to the administration of the combination. In some embodiments, the T cells are CD4+ and/or CD8⁺ T cells.

In some embodiments of the methods of the present disclosure, activated CD4+ and/or CD8⁺ T-cells in the individual are characterized by IFN-γ producing CD4⁺ and/or CD8⁺ T cells and/or enhanced cytolytic activity as compared to before the administration of the combination. IFN-γ may be measured by any means known in the art, including, e.g., intracellular cytokine staining (ICS) involving cell fixation, permeabilization, and staining with an antibody against IFN-γ. Cytolytic activity may be measured by any means known in the art, e.g., using a cell killing assay with mixed effector and target cells.

In some embodiments, CD8⁺ T-cells are characterized, e.g., by presence of CD8b expression (e.g., by RT-PCR using e.g., Fluidigm) (Cd8b is also known as T-cell surface glycoprotein CD8 beta chain; CD8 antigen, alpha polypeptide p3′7; Accession No. is NM_172213). In some embodiments, CD8⁺ T cells are from peripheral blood. In some embodiments, CD8⁺ T cells are from tumor.

In some embodiments, Treg cells are characterized, e.g., by presence of Fox3p expression (e.g., by RT-PCR e.g., using Fluidigm) (Foxp3 is also known as Forkhead box protein P3; scurfin; FOXP3delta7; immunodeficiency, polyendocrinopathy, enteropathy, X-linked; the accession no. is NM_014009). In some embodiments, Treg are from peripheral blood. In some embodiments, Treg cells are from tumor.

In some embodiments, inflammatory or activated T-cells are characterized, e.g., by presence of TBET and/or CXCR3 expression or by a TBET:EOMES ratio that correlates with inflammatory or activated T-cells (e.g., by RT-PCR using, e.g., Fluidigm). In some embodiments, inflammatory or activated T cells are from peripheral blood. In some embodiments, inflammatory or activated T cells are from tumor. [symbol] some embodiments of the methods of the present discl [symbol] D8⁺ T cells exhibit increased release of cytokines selected from the group consisting of IFN-γ, TNF-α. Cytokine release may be measured by any means known in the art, e.g., using Western blot, ELISA, or immunohistochemical assays to detect the presence of released cytokines in a sample containing CD4⁺ and/or CD8⁺ T-cells.

In some embodiments of the methods of the present disclosure, the CD4⁺ and/or CD8⁺ T cells are effector memory T cells. In some embodiments of the methods of the present disclosure, the CD4⁺ and/or CD8⁺ effector memory T cells are characterized by having the expression of CD44^{high} CD62^{low}. Expression of CD44^{high} CD62^{low} may be detected by any means known in the art, e.g., by preparing single cell suspensions of tissue (e.g., a cancer tissue) and performing surface staining and flow cytometry using commercial antibodies against CD44 and CD62L. In some embodiments of the methods of the present disclosure, the CD4⁺ and/or CD8+ effector memory T cells are characterized by having expression of CXCR3 (also known as C—X—C chemokine receptor type 3; Mig receptor; IP10 receptor; G protein-coupled receptor 9; interferon-inducible protein 10 receptor; Accession No. NM_001504). In some embodiments, the CD4⁺ and/or CD8⁺ effector memory T cells are from peripheral blood. In some embodiments, the CD4⁺ and/or CD8⁺ effector memory T cells are from tumor.

In some embodiments of the methods of the present disclosure, the administration of an effective amount of a LSD inhibitor and a PD-1 binding antagonist and optionally an ancillary agent such as a chemotherapeutic agent to an individual is characterized by increased levels of inflammatory markers (e.g., CXCR3) on CD8⁺ T cells as compared to before administration of the combination therapy. CXCR3/CD8⁺ T cells may be measured by any means known the art. In some embodiments, CXCR3/CD8⁺ T cells are from peripheral blood. In some embodiments, CXCR3/CD8+ T cells are from tumor.

In some embodiments of the methods of the invention, Treg function is suppressed as compared to before administration of the combination. In some embodiments, T-cell exhaustion is decreased as compared to before administration of the combination.

In some embodiments, number of Treg is decreased as compared to before administration of the combination. In some embodiments, the levels of plasma IFN-γ is increased as compared to before administration of the combination. Treg number may be assessed, e.g., by determining percentage of CD4⁺Fox3p⁺CD45⁺ cells (e.g., by FACS analysis). In some embodiments, absolute number of Treg, e.g., in a sample, is determined. In some embodiments, Treg are from peripheral blood. In some embodiments, Treg are from tumor.

In some embodiments, T-cell priming, activation and/or proliferation is increased as compared to before administration of the combination. In some embodiments, the T-cells are CD4⁺ and/or CD8⁺ T cells. In some embodiments, T-cell proliferation is detected by determining percentage of Ki67⁺CD8⁺ T cells (e.g., by FACS analysis). In some embodiments, T-cell proliferation is detected by determining percentage of Ki67⁺CD4⁺ T cells (e.g., by FACS analysis). In some embodiments, the T-cells are from peripheral blood. In some embodiments, the T-cells are from tumor.

### 5. Methods of Detection and Diagnosis

In accordance with the present invention, nuclear LSD and EOMES can be employed as biomarkers of T-cell mesenchymal phenotype and impaired T-cell function. Additionally, PD-1 and TBET may be used as known in the art to assess T-cell exhaustion. T-cells can be obtained [symbol] ining patient samples which are suitably selected from tis [symbol] ; tumors and fluid samples such as peripheral blood. In some embodiments, the sample is obtained prior to treatment with the therapeutic combination. In some embodiments, the tissue sample is formalin fixed and paraffin embedded, archival, fresh or frozen. In some embodiments, the sample is whole blood. In some embodiments, the whole blood comprises immune cells, circulating tumor cells and any combinations thereof.

Presence and/or expression levels/amount of a biomarker (e.g., any one or more of LSD (e.g., LSD1, nuclear LSD, etc.), EOMES, TBET and PD-1, also referred to herein collectively as “T-cell function biomarkers”) can be determined qualitatively and/or quantitatively based on any suitable criterion known in the art, including but not limited to DNA, mRNA, cDNA, proteins, protein fragments and/or gene copy number. In certain embodiments, presence and/or expression levels/amount of a biomarker in a first sample is increased or elevated as compared to presence/absence and/or expression levels/amount in a second sample (e.g., before treatment with the therapeutic combination). In certain embodiments, presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue. Additional disclosures for determining presence/absence and/or expression levels/amount of a gene are described herein.

In some embodiments of any of the methods, elevated expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5-fold, about 1.75-fold, about-2 fold, about 2.25-fold, about 2.5-fold, about 2.75-fold, about 3.0-fold, or about 3.25-fold as compared to a reference sample, reference cell, reference tissue, control sample, control cell, control tissue, or internal control (e.g., housekeeping gene).

In some embodiments of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

Presence and/or expression level/amount of various biomarkers in a sample can be analyzed by a number of methodologies, many of which are known in the art and understood by the skilled artisan, including, but not limited to, immunohistochemistry (“IHC”), Western blot [symbol] oprecipitation, molecular binding assays, ELISA, ELIFA, flu [symbol] cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments, presence and/or expression level/amount of a biomarker is determined using a method comprising: (a) performing gene expression profiling, PCR (such as rtPCR or qRT-PCR), RNA-seq, microarray analysis, SAGE, MassARRAY technique, or FISH on a sample (such as a subject cancer sample); and b) determining presence and/or expression level/amount of a biomarker in the sample. In some embodiments, the microarray method comprises the use of a microarray chip having one or more nucleic acid molecules that can hybridize under stringent conditions to a nucleic acid molecule encoding a gene mentioned above or having one or more polypeptides (such as peptides or antibodies) that can bind to one or more of the proteins encoded by the genes mentioned above. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

Samples from mammals can be conveniently assayed for mRNAs using Northern, dot blot or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of anti-angiogenic therapy may be arrayed on a solid support. Hybridization of a labeled probe with a particular array member indicates that the sample from which the probe was derived expresses that gene. [symbol] ccording to some embodiments, presence and/or express [symbol] measured by observing protein expression levels of an aforementioned gene. In certain embodiments, the method comprises contacting the biological sample with antibodies to a biomarker (e.g., anti-PD-1 antibodies, anti-LSD antibodies, anti-TBET antibodies, anti-EOMES antibodies) described herein under conditions permissive for binding of the biomarker, and detecting whether a complex is formed between the antibodies and the biomarker. Such method may be an in vitro or in vivo method. In some embodiments, one or more anti-biomarker antibodies are used to select subjects eligible for combination therapy with a LSD inhibitor and a PD-1 binding antagonist.

In certain embodiments, the presence and/or expression level/amount of biomarker proteins in a sample is examined using IHC and staining protocols. IHC staining of tissue sections has been shown to be a reliable method of determining or detecting presence of proteins in a sample. In some embodiments, expression of a T-cell function biomarker in a sample from an individual is elevated protein expression and, in further embodiments, is determined using IHC. In one embodiment, expression level of biomarker is determined using a method comprising: (a) performing IHC analysis of a sample (such as a subject cancer sample) with an antibody; and b) determining expression level of a biomarker in the sample. In some embodiments, IHC staining intensity is determined relative to a reference. In some embodiments, the reference is a reference value. In some embodiments, the reference is a reference sample (e.g., control cell line staining sample or tissue sample from non-cancerous patient).

In some embodiments, T-cell function biomarker expression is evaluated on a tumor or tumor sample. As used herein, a tumor or tumor sample may encompass part or all of the tumor area occupied by tumor cells. In some embodiments, a tumor or tumor sample may further encompass tumor area occupied by tumor associated intratumoral cells and/or tumor associated stroma (e.g., contiguous peri-tumoral desmoplastic stroma). Tumor associated intratumoral cells and/or tumor associated stroma may include areas of immune infiltrates (e.g., tumor infiltrating immune cells as described herein) immediately adjacent to and/or contiguous with the main tumor mass. In some embodiments, T-cell function biomarker expression is evaluated on tumor cells. In some embodiments, T-cell function biomarker expression is evaluated on immune cells within the tumor area as described above, such as tumor infiltrating immune cells.

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Presence and/or expression level/amount of a selected T-cell function biomarker in a tissue or cell sample may also be examined by way of functional or activity-based assays. For instance, if the biomarker is an enzyme (e.g., LSD), one may conduct assays (e.g., demethylase assays) known in the art to determine or detect the presence of the given enzymatic activity in the tissue or cell sample.

[symbol] certain embodiments, the samples are normalized for b [symbol] amount of the biomarker assayed and variability in the quality of the samples used, and variability between assay runs. Such normalization may be accomplished by detecting and incorporating the expression of certain normalizing biomarkers, including well known housekeeping genes. Alternatively, normalization can be based on the mean or median signal of all of the assayed genes or a large subset thereof (global normalization approach). On a gene-by-gene basis, measured normalized amount of a subject tumor mRNA or protein is compared to the amount found in a reference set. Normalized expression levels for each mRNA or protein per tested tumor per subject can be expressed as a percentage of the expression level measured in the reference set. The presence and/or expression level/amount measured in a particular subject sample to be analyzed will fall at some percentile within this range, which can be determined by methods well known in the art.

In some embodiments, the sample is a clinical sample. In other embodiments, the sample is used in a diagnostic assay. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Genes or gene products can be detected from cancer or tumor tissue or from other body samples such as urine, sputum, serum or plasma. The same techniques discussed above for detection of target genes or gene products in cancerous samples can be applied to other body samples. Cancer cells may be sloughed off from cancer lesions and appear in such body samples. By screening such body samples, a simple early diagnosis can be achieved for these cancers. In addition, the progress of therapy can be monitored more easily by testing such body samples for target genes or gene products.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combination of multiple samples from one or more healthy individuals who are not the subject or individual. In certain embodiments, a reference sample, reference cell, reference tissue, control sample, control cell, or control tissue is a combination of multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the subject or individual. In certain embodiments, a reference sample, reference cell, reference tissue, control sample, control cell, or control tissue is pooled RNA samples from tumor [symbol] plasma or serum samples from one or more individuals [symbol] rder (e.g., cancer) who are not the subject or individual.

In some embodiments, the sample is a tissue sample from the individual. In some embodiments, the tissue sample is a tumor tissue sample (e.g., biopsy tissue). In some embodiments, the tissue sample is lung tissue. In some embodiments, the tissue sample is renal tissue. In some embodiments, the tissue sample is skin tissue. In some embodiments, the tissue sample is pancreatic tissue. In some embodiments, the tissue sample is gastric tissue. In some embodiments, the tissue sample is bladder tissue. In some embodiments, the tissue sample is esophageal tissue. In some embodiments, the tissue sample is mesothelial tissue. In some embodiments, the tissue sample is breast tissue. In some embodiments, the tissue sample is thyroid tissue. In some embodiments, the tissue sample is colorectal tissue. In some embodiments, the tissue sample is head and neck tissue. In some embodiments, the tissue sample is osteosarcoma tissue. In some embodiments, the tissue sample is prostate tissue. In some embodiments, the tissue sample is ovarian tissue, HCC (liver), blood cells, lymph nodes, and/or bone/bone marrow tissue. In some embodiments, the tissue sample is colon tissue. In some embodiments, the tissue sample is endometrial tissue. In some embodiments, the tissue sample is brain tissue (e.g., glioblastoma, neuroblastoma, and so forth).

In some embodiments, a tumor tissue sample (the term “tumor sample” is used interchangeably herein) may encompass part or all of the tumor area occupied by tumor cells. In some embodiments, a tumor or tumor sample may further encompass tumor area occupied by tumor associated intratumoral cells and/or tumor associated stroma (e.g., contiguous peri-tumoral desmoplastic stroma). Tumor associated intratumoral cells and/or tumor associated stroma may include areas of immune infiltrates (e.g., tumor infiltrating immune cells as described herein) immediately adjacent to and/or contiguous with the main tumor mass.

In some embodiments, tumor cell staining is expressed as the percent of all tumor cells showing membranous staining of any intensity. Infiltrating immune cell staining may be expressed as the percent of the total tumor area occupied by immune cells that show staining of any intensity. The total tumor area encompasses the malignant cells as well as tumor-associated stroma, including areas of immune infiltrates immediately adjacent to and contiguous with the main tumor mass. In addition, infiltrating immune cell staining may be expressed as the percent of all tumor infiltrating immune cells.

In some embodiments of any of the methods, the disease or disorder is a tumor. In some embodiments, the tumor is a malignant cancerous tumor (i.e., cancer). In some embodiments, the tumor and/or cancer is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, [symbol] n, uterus, male genital organ, urinary organs (e.g., uroth [symbol] dysplastic urothelium carcinoma, transitional cell carcinoma), bladder, and skin. Solid tumors of non-epithelial origin include sarcomas, brain tumors, and bone tumors. In some embodiments, the cancer is non-small cell lung cancer (NSCLC). In some embodiments, the cancer is second-line or third-line locally advanced or metastatic non-small cell lung cancer. In some embodiments, the cancer is adenocarcinoma. In some embodiments, the cancer is squamous cell carcinoma. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma. In some embodiments, the cancer is a primary tumor. In some embodiments, the cancer is a metastatic tumor at a second site derived from any of the above types of cancer.

In some embodiments of any of the methods, the cancer displays human effector cells (e.g., is infiltrated by human effector cells). Methods for detecting human effector cells are well known in the art, including, e.g., by IHC. In some embodiments, the cancer displays high levels of human effector cells. In some embodiments, human effector cells are one or more of NK cells, macrophages, monocytes. In some embodiments, the cancer is any cancer described herein. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma.

In some embodiments of any of the methods, the cancer displays cells expressing FcR (e.g., is infiltrated by cells expressing FcR). Methods for detecting FcR are well known in the art, including, e.g., by IHC. In some embodiments, the cancer displays high levels of cells expressing FcR. In some embodiments, FcR is FcγR. In some embodiments, FcR is activating FcγR. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma.

In some embodiments, the T-cell function biomarker is detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof. In some embodiments, the T-cell function biomarker is detected using FACS analysis. In some embodiments, the T-cell function biomarker is PD-1. In some embodiments, the PD-1 expression is detected in blood samples. In some embodiments, the PD-1 expression is detected on circulating immune cells in blood samples. In some embodiments, the circulating immune cell is a CD3⁺/CD8⁺ T cell. In some embodiments, prior to analysis, the immune cells are isolated from the blood samples. Any suitable method to isolate/enrich such population of cells may be used including, but not limited to, cell sorting. In some embodiments, the PD-1 expression is reduced in samples from individuals that respond to treatment with a LSD inhibitor and/or PD-1 binding antagonist, such as an anti-PD-1 antibody. In some embodiments, the PD-1 expression is elevated on circulating immune cells, such as CD3⁺/CD8⁺ T cells, in blood samples.

Also provided herein are diagnostic methods and kits that are based on the determination that LSD and EOMES co-localize in the nucleus and that this co-localization [symbol] st in part to EMT of T-cells and repression of their immun [symbol] diagnostic methods suitably comprise: (i) obtaining a sample from a subject, wherein the sample comprises a T-cell (e.g., CD8⁺ T-cell or CD4⁺ T-cell); (ii) contacting the sample with a first binding agent that binds to LSD (e.g., LSD1, nuclear LSD, etc.) in the sample and a second binding agent that binds to EOMES in the sample; and (iii) detecting localization of the first and second binding agents in the nucleus of the T-cell, wherein localization of the first and second binding agents in the nucleus of the T-cell is indicative of the presence of a T-cell dysfunctional disorder in the subject.

The first and second binding-agents suitably bind to epitopes of LSD (e.g., LSD1, nuclear LSD, etc.) and EOMES polypeptides, respectively. Any suitable epitope may be chosen in the amino acid sequence of LSD (as set forth for example in GenPept Accession Nos. NP_055828.2, NP_001009999.1, 060341.2 and NP_694587.3), or in the amino acid sequence of EOMES (as set forth for example in GenPept Accession Nos. NP_001265111, NP_005433 and NP_001265112).

Localization of LSD and EOMES in the nucleus of the T-cell may be performed using any suitable localization technique, e.g., by IHC, typically using an anti-LSD antibody that has a different detectable moiety or label than an anti-EOMES antibody. In some embodiments, spatial proximity assays (also referred to as “proximity assays”) are employed, which can be used to assess the formation of a complex between LSD and EOMES. Proximity assays rely on the principle of “proximity probing”, wherein an analyte, typically an antigen, is detected by the coincident binding of multiple (i.e., two or more, generally two, three or four) binding agents or probes, which when brought into proximity by binding to the analyte (hence “proximity probes”) allow a signal to be generated.

In some embodiments, at least one of the proximity probes comprises a nucleic acid domain (or moiety) linked to the analyte-binding domain (or moiety) of the probe, and generation of the signal involves an interaction between the nucleic acid moieties and/or a further functional moiety which is carried by the other probe(s). Thus signal generation is dependent on an interaction between the probes (more particularly by the nucleic acid or other functional moieties/domains carried by them) and hence only occurs when both the necessary two (or more) probes have bound to the analyte, thereby lending improved specificity to the detection system. The concept of proximity probing has been developed in recent years and many assays based on this principle are now well known in the art.

Proximity assays are typically used to assess whether two particular proteins or portions thereof are in close proximity, e.g., proteins that are bound to each other, fusion proteins, and/or proteins that are positioned in close proximity. One such assay, known as proximity ligation assay (PLA), and which is used in some embodiments of the present invention, features two antibodies (raised in different species) bound to the targets of interest (see Nature Methods 3, 995-1000 (2006)). PLA probes, which are species-specific secondary antibodies with a unique oligonucleotide strand attached, are then bound to the appropriate primary antibodies. In the case of the targets being in close proximity, the oligonucleotide strands of the PLA probes can interact with additional ssDNA and DNA ligase such they can be circulated and amplified via rolling circle amplification (RCA). When highly processive DNA polymerases such as Phi29 DNA polymerase is used, the circular DNA template can be replicated hundreds to thousands of times longer and as a result producing ssDNA molecules from hundreds of nanometers to microns in length (see, Angewandte Chemie International Edition, 2008, 47, 6330-6337). After the amplification, the [symbol] an be detected via detection systems. Thus, a visible sign [symbol] he targets of interest are in close proximity. These assays feature the use of several DNA-antibody conjugates as well as enzymes such as DNA ligase and DNA polymerase.

In other embodiments, a dual binders (DB) assay is employed, which utilizes a bispecific detection agent consisting of two Fab fragments with fast off-rate kinetics joined by a flexible linker (Van dieck et al., 2014 Chemistry & Biology Vol. 21(3):357-368). In principle, because the dual binders comprise Fab fragments with fast off-rate kinetics, the dual binders are washed off if only one of the Fab fragments is bound to its epitope (simultaneous cooperative binding of both Fab fragments of the dual binder prevents dissociation of the dual binder and leads to positive staining/visibility).

According to another approach disclosed in International Publication WO2014/139980, which is encompassed in the practice of the present invention, proximity assays and tools are described, which employ a biotin ligase substrate and an enzyme to perform a proximity assay. The method provides detection of target molecules and proximity while maintaining the cellular context of the sample. The use of biotin ligase such as an enzyme from E. coli and peptide substrate such as amino-acid substrate for that enzyme provides for a sensitive and specific detection of protein-protein interactions in FFPE samples. Because biotin ligase can efficiently biotinylate appropriate peptide substrate in the presence of biotin and the reaction can only occur when the enzyme makes physical contact with the peptide substrate, biotin ligase and the substrate can be separately conjugated to two antibodies that recognize targets of interest respectively.

Also provided herein are methods for monitoring pharmacodynamic activity of a PD-1 binding antagonist treatment by measuring the expression level of one or more T-cell function biomarkers as described herein in a sample comprising leukocytes obtained from the subject, where the subject has been treated with a PD-1 binding antagonist and a LSD inhibitor, and where the one or more T-cell function biomarkers are selected from nuclear LSD (e.g., LSD1, nuclear LSD, etc.), TBET, PD-1 and EOMES, and determining the treatment as demonstrating pharmacodynamic activity based on the expression level of the one or more T-cell function biomarkers in the sample obtained from the subject, as compared with a reference, where an increased expression level of the one or more T-cell function biomarkers as compared with the reference indicates pharmacodynamic activity to the PD-1 antagonist treatment. These methods may further comprise measuring the expression level of one or more additional biomarkers of T cell function and/or cellular composition (e.g., percentage of Treg and/or absolute number of Treg; e.g., number of CD8⁺ or CD4⁺ effector T cells), wherein the additional biomarkers of T cell function include a cytokine, e.g., IFN-γ, a T cell marker, or a memory T cell marker (e.g., a marker of T effector memory cells); and determining the treatment as demonstrating pharmacodynamic activity based on the expression level of the one or more T-cell function biomarkers, the one or more additional biomarkers of T cell function and/or cellular composition in the sample obtained from the subject, as compared with a reference, where an increased expression level of the one or more T-cell function biomarkers, the one or more additional biomarkers of T cell function and/or cellular composition as compared with the reference indicates pharmacodynamic activity to the PD-1 antagonist treatment. Expression level of the biomarker(s) and/or cellular composition may be measured by one or more methods as described herein. [symbol] s used herein, “pharmacodynamic (PD) activity” may refe [symbol] treatment (e.g., a LSD inhibitor in combination with a PD-1 binding antagonist treatment and optionally a chemotherapeutic agent) to the subject. An example of a PD activity may include modulation of the expression level of one or more genes. Without wishing to be bound to theory, it is thought that monitoring PD activity, such as by measuring expression of one or more T-cell function biomarkers, may be advantageous during a clinical trial examining a LSD inhibitor and PD-1 binding antagonist and optionally a chemotherapeutic agent. Monitoring PD activity may be used, for example, to monitor response to treatment, toxicity, and the like.

In some embodiments, the expression level of one or more marker genes, proteins and/or cellular composition may be compared to a reference which may include a sample from a subject not receiving a treatment (e.g., a LSD inhibitor treatment in combination with a PD-1 binding antagonist and optionally a chemotherapeutic agent). In some embodiments, a reference may include a sample from the same subject before receiving a treatment (e.g., a LSD inhibitor treatment in combination with a PD-1 binding antagonist and optionally a chemotherapeutic agent). In some embodiments, a reference may include a reference value from one or more samples of other subjects receiving a treatment (e.g., a LSD inhibitor treatment in combination with a PD-1 binding antagonist and optionally a chemotherapeutic agent). For example, a population of patients may be treated, and a mean, average, or median value for expression level of one or more genes may be generated from the population as a whole. A set of samples obtained from cancers having a shared characteristic (e.g., the same cancer type and/or stage, or exposure to a common treatment such as a LSD inhibitor treatment in combination with a PD-1 binding antagonist and optionally a chemotherapeutic agent) may be studied from a population, such as with a clinical outcome study. This set may be used to derive a reference, e.g., a reference number, to which a subject's sample may be compared. Any of the references described herein may be used as a reference for monitoring PD activity.

Certain aspects of the present disclosure relate to measurement of the expression level of one or more biomarkers (e.g., gene expression products including mRNAs and proteins) in a sample. In some embodiments, a sample may include leukocytes. In some embodiments, the sample may be a peripheral blood sample (e.g., from a patient having a tumor). In some embodiments, the sample is a tumor sample. A tumor sample may include cancer cells, lymphocytes, leukocytes, stroma, blood vessels, connective tissue, basal lamina, and any other cell type in association with the tumor. In some embodiments, the sample is a tumor tissue sample containing tumor-infiltrating leukocytes. In some embodiments, the sample may be processed to separate or isolate one or more cell types (e.g., leukocytes). In some embodiments, the sample may be used without separating or isolating cell types.

A tumor sample may be obtained from a subject by any method known in the art, including without limitation a biopsy, endoscopy, or surgical procedure. In some embodiments, a tumor sample may be prepared by methods such as freezing, fixation (e.g., by using formalin or a similar fixative), and/or embedding in paraffin wax. In some embodiments, a tumor sample may be sectioned. In some embodiments, a fresh tumor sample (i.e., one that has not been prepared by the methods described above) may be used. In some embodiments, a tumor sample may be prepared by incubation in a solution to preserve mRNA and/or protein integrity.

In some embodiments, the sample may be a peripheral blood sample. A peripheral blood sample may include white blood cells, PBMCs, and the like. Any technique known in the art [symbol] ocytes from a peripheral blood sample may be used. For [symbol] mple may be drawn, red blood cells may be lysed, and a white blood cell pellet may be isolated and used for the sample. In another example, density gradient separation may be used to separate leukocytes (e.g., PBMCs) from red blood cells. In some embodiments, a fresh peripheral blood sample (i.e., one that has not been prepared by the methods described above) may be used. In some embodiments, a peripheral blood sample may be prepared by incubation in a solution to preserve mRNA and/or protein integrity.

In some embodiments, responsiveness to treatment may refer to any one or more of: extending survival (including overall survival and progression free survival); resulting in an objective response (including a complete response or a partial response); or improving signs or symptoms of cancer. In some embodiments, responsiveness may refer to improvement of one or more factors according to the published set of RECIST guidelines for determining the status of a tumor in a cancer patient, i.e., responding, stabilizing, or progressing. For a more detailed discussion of these guidelines, see, Eisenhauer et al. (2009 Eur J Cancer 45: 228-47), Topalian et al. (2012 N Engl J Med 366:2443-54), Wolchok et al. (2009 Clin Can Res 15:7412-20) and Therasse et al. (2000 J. Natl. Cancer Inst. 92:205-16). A responsive subject may refer to a subject whose cancer(s) show improvement, e.g., according to one or more factors based on RECIST criteria. A non-responsive subject may refer to a subject whose cancer(s) do not show improvement, e.g., according to one or more factors based on RECIST criteria.

Conventional response criteria may not be adequate to characterize the anti-tumor activity of therapeutic agents of the invention, which can produce delayed responses that may be preceded by initial apparent radiological progression, including the appearance of new lesions. Therefore, modified response criteria have been developed that account for the possible appearance of new lesions and allow radiological progression to be confirmed at a subsequent assessment. Accordingly, in some embodiments, responsiveness may refer to improvement of one of more factors according to immune-related response criteria (irRC). See, e.g., Wolchok et al. (2009, supra). In some embodiments, new lesions are added into the defined tumor burden and followed, e.g., for radiological progression at a subsequent assessment. In some embodiments, presence of non-target lesions is included in assessment of complete response and not included in assessment of radiological progression. In some embodiments, radiological progression may be determined only on the basis of measurable disease and/or may be confirmed by a consecutive assessment ≥4 weeks from the date first documented.

In some embodiments, responsiveness may include immune activation. In some embodiments, responsiveness may include treatment efficacy. In some embodiments, responsiveness may include immune activation and treatment efficacy.

### 6. Kits

In other embodiments of the invention, therapeutic kits are provided comprising a LSD inhibitor (e.g., a LSD1 inhibitor, a nuclear LSD inhibitor, etc.) and a PD-1 binding antagonist. In some embodiments, the therapeutic kits further comprise a package insert comprising instructional material for administering concurrently the LSD inhibitor and the PD-1 binding antagonist to treat a T-cell dysfunctional disorder, or to enhance immune function (e.g., immune effector function, T-cell function etc.) in an individual having cancer, or to treat or delay cancer progression, or to treat infection in an individual. In some embodiments, the therapeutic kits may further comprise a chemotherapeutic agent (e.g., an agent that targets rapidly dividing cells and/or [symbol] cle or cell division, representative examples of which incl [symbol] compounds such as a taxane). Any of LSD inhibitor, PD-1 binding antagonist and optionally chemotherapeutic agents described herein or known in the art may be included in the kits.

In some embodiments, the LSD inhibitor, PD-1 binding antagonist and optionally chemotherapeutic agents are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The kits may further include other materials desirable from a commercial and user standpoint, including other buffers, diluents, filters, needles, syringes, and package inserts with instructional material for use. In some embodiments, the kits further include one or more of other agents (e.g., a chemotherapeutic agent, and anti-neoplastic agent). Suitable containers for the one or more agent include, for example, bottles, vials, bags and syringes.

In other embodiments of the invention, diagnostic kits are provided for determining expression of biomarkers, including the T-cell function biomarkers disclosed herein, which include reagents that allow detection and/or quantification of the biomarkers. Such reagents include, for example, compounds or materials, or sets of compounds or materials, which allow quantification of the biomarkers. In specific embodiments, the compounds, materials or sets of compounds or materials permit determining the expression level of a gene (e.g., T-cell function biomarker gene), including without limitation the extraction of RNA material, the determination of the level of a corresponding RNA, etc., primers for the synthesis of a corresponding cDNA, primers for amplification of DNA, and/or probes capable of specifically hybridizing with the RNAs (or the corresponding cDNAs) encoded by the genes, TaqMan probes, proximity assay probes, ligases, antibodies etc.

The kits may also optionally include appropriate reagents for detection of labels, positive and negative controls, washing solutions, blotting membranes, microtiter plates, dilution buffers and the like. For example, a nucleic acid-based detection kit may include (i) a T-cell function biomarker polynucleotide (which may be used as a positive control), (ii) a primer or probe that specifically hybridizes to a T-cell function biomarker polynucleotide. Also included may be enzymes suitable for amplifying nucleic acids including various polymerases (reverse transcriptase, Taq, Sequenase™, DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) a T-cell function biomarker polypeptide (which may be used as a positive control), (ii) an antibody that binds specifically to a T-cell function biomarker polypeptide. The kit can also feature various devices (e.g., one or more) and reagents (e.g., one or more) for performing one of the assays described herein; and/or printed instructional material for using the kit to quantify the expression of a T-cell function biomarker gene. The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein. [symbol] aterials suitable for packing the components of the diagn [symbol] e crystal, plastic (polyethylene, polypropylene, polycarbonate and the like), bottles, vials, paper, envelopes and the like. Additionally, the kits of the invention can contain instructional material for the simultaneous, sequential or separate use of the different components contained in the kit. The instructional material can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain Internet addresses that provide the instructional material.

In order that the invention may be readily understood and put into practical effect, particular preferred embodiments will now be described by way of the following non-limiting examples.

### EXAMPLES

### Example 1

### Dual Epigenetic-Immunotherapy Inhibits Tumor Burden and Mesenchymal, Stem-Like CTC Signature

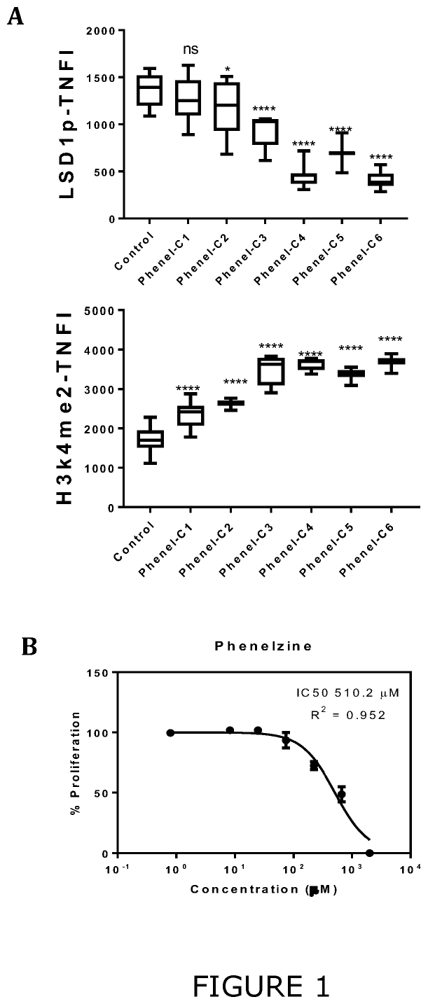

The present inventors examined the LSD1 inhibitor, phenelzine sulfate, in the triple negative, breast cancer cell line MDA-MB-231 to test its efficacy in inhibiting demethylation and cell proliferation. It was observed that phenelzine sulfate inhibited the nuclear axis of LSD1, as measured by H3k4me2 de-methylation as well as inhibiting the proliferation of MDA-MB-231, as measured by the WST-1 assay (FIG. 1A, B).

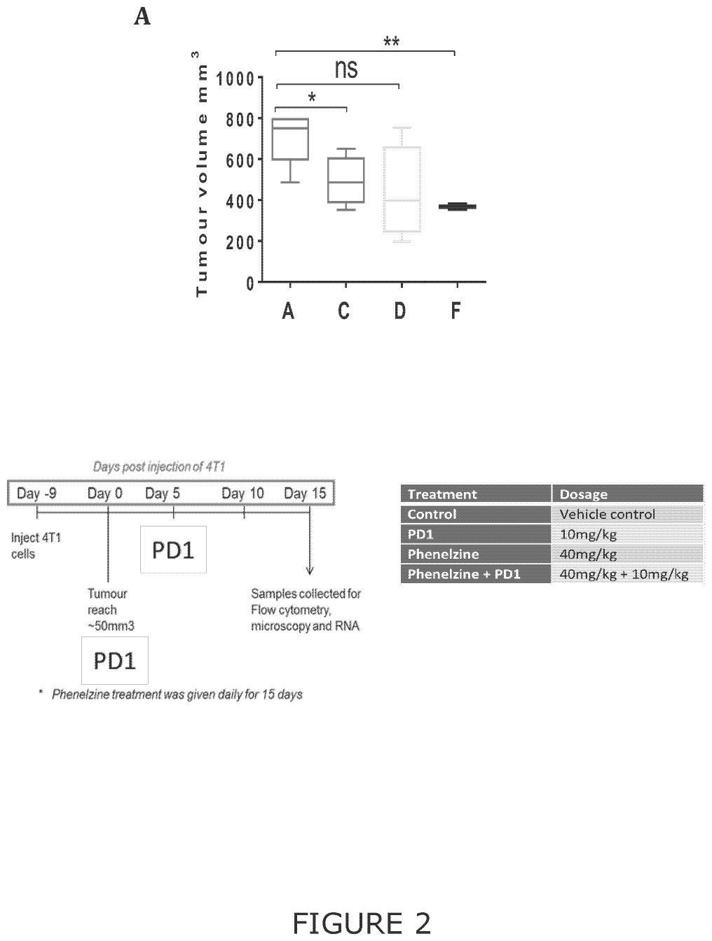

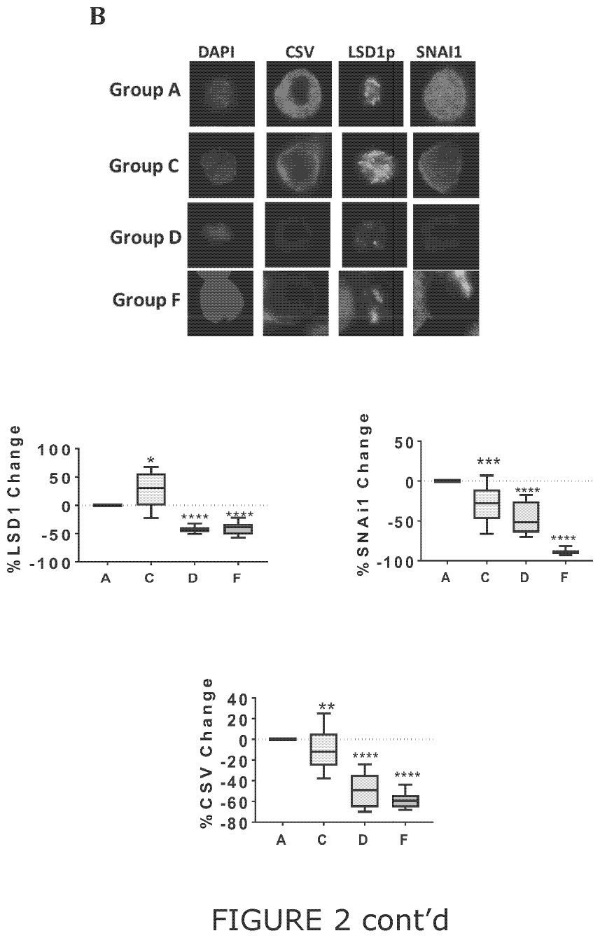

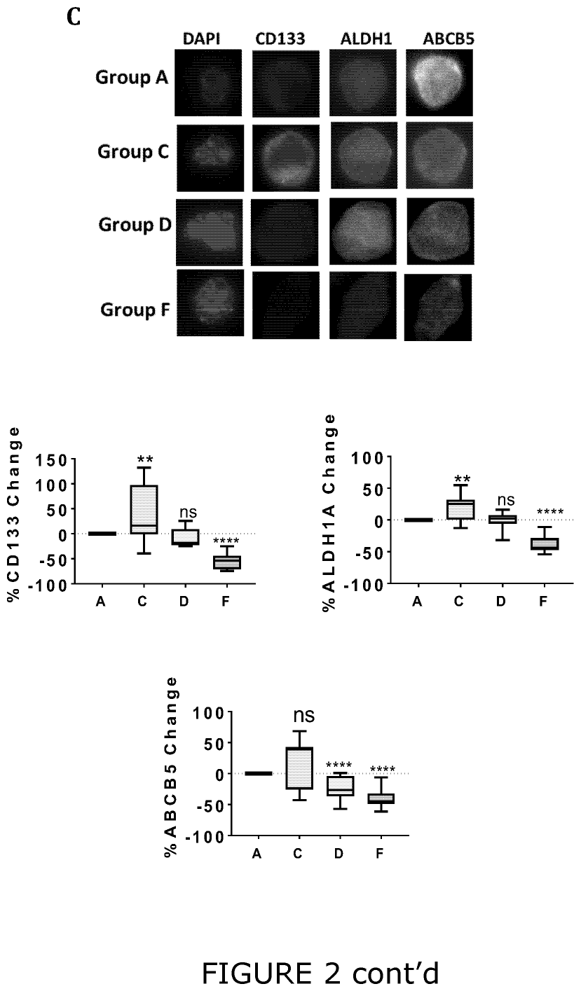

Next, the effect of phenelzine sulfate was examined in the 4T1 mouse metastatic breast cancer model in the context of PD1 immunotherapy. 4T1 mice were treated with vehicle control (Group A), anti-PD1 antibody (10 mg/kg) (Group C), phenelzine sulfate (40 mg/kg) (Group D) or a combination of both (Group F). It was found that all treatments significantly reduced the primary tumor volume (FIG. 2A).

Cancer cells harvested from the primary tumor micro-environment (TME) were examined for % change in protein expression relative to vehicle control via immunofluorescence (IF). The expression of a mesenchymal circulating tumor cell (CTC) signature biomarker panel (CSV, LSD1p, SNAI1) on the cancer cells was assessed and notably, it was found that anti-PD1 immunotherapy alone moderately increased nuclear expression of LSD1, had little effect on expression of CSV and strongly inhibited SNAI1 expression (FIG. 2B, Group C). By contrast, phenelzine sulfate exhibited strong inhibition on LSD1, CSV and SNAI1 expression (FIG. 2B, Group D). The combination therapy overall had the strongest % inhibition of this mesenchymal signature panel (FIG. 2B, Group F).

Next, the cancer cells were assessed for expression of a chemo-resistant, stem-like biomarker signature biomarker panel (CD133, ALDH1A and ABCB5). The % change in expression in the anti-PD1 treated group showed a moderate increase in expression of ALDH1A and CD133 but no change in expression of ABCB5 (FIG. 2C, Group C). Additionally, phenelzine sulfate alone did not significantly affect expression of CD133 and ALDHA1 but strongly inhibited ABCB5 expression (FIG. 2C, Group D). Strikingly, the combination therapy significantly abrogated all 3 biomarkers of this stem-like, chemo-resistant signature panel (FIG. 2C, Group F).

### Example 2

### Dual Epigenetic-Immunotherapy Inhibits Metastatic Progression in 4T1 Mouse Model

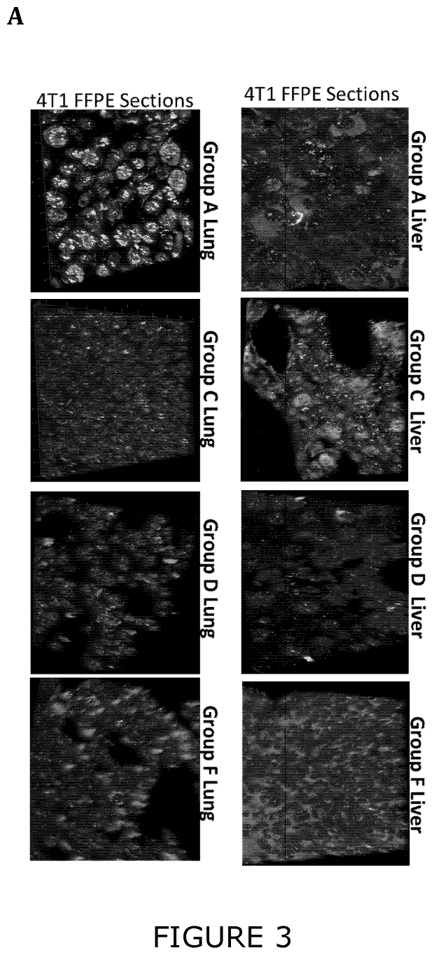

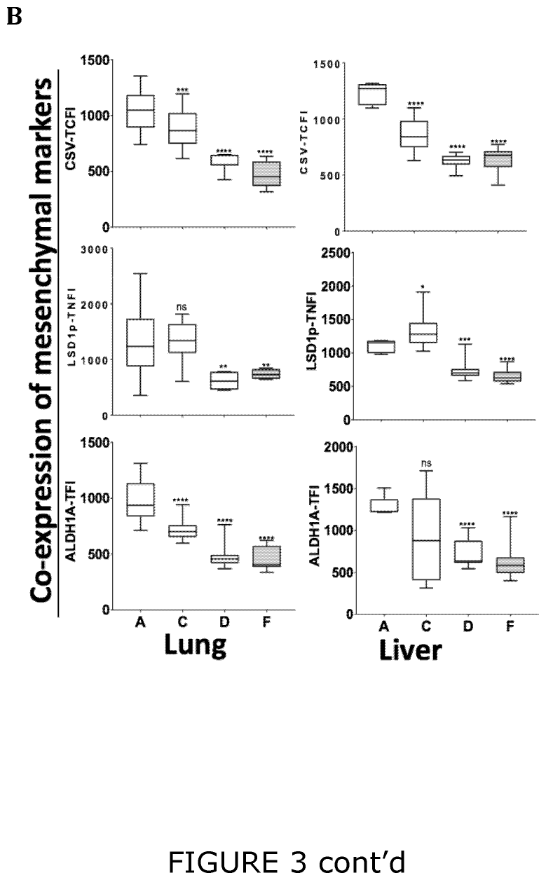

The expression of a mesenchymal, stem-like biomarker panel was examined in metastatic sites in a 4T1 mouse model treated with vehicle control (Group A), anti-PD1 antibody (10 mg/kg) (Group C), phenelzine sulfate (40 mg/kg) (Group D) or a combination of both (Group F). The present inventors examined the presence of metastatic lesions in the lung or liver and measured the immunofluorescence (IF) of formalin-fixed paraffin-embedded (FFPE) tissue examining the IF intensity of LSD1p, CSV and ALDH1A. Notably, they found that overall, PD1 inhibition alone had a relatively minor effect on metastatic lesion cancer cells, although it was observed that inhibiting PD1 expression resulted in inhibition of CSV expression in liver and lung lesions and inhibition of ALDH1A in lung lesions (FIG. 3, Group C). By contrast both phenelzine sulfate alone and the combination therapy strongly inhibited all 3 markers in both liver and lung lesions (FIG. 3, Groups D and F).

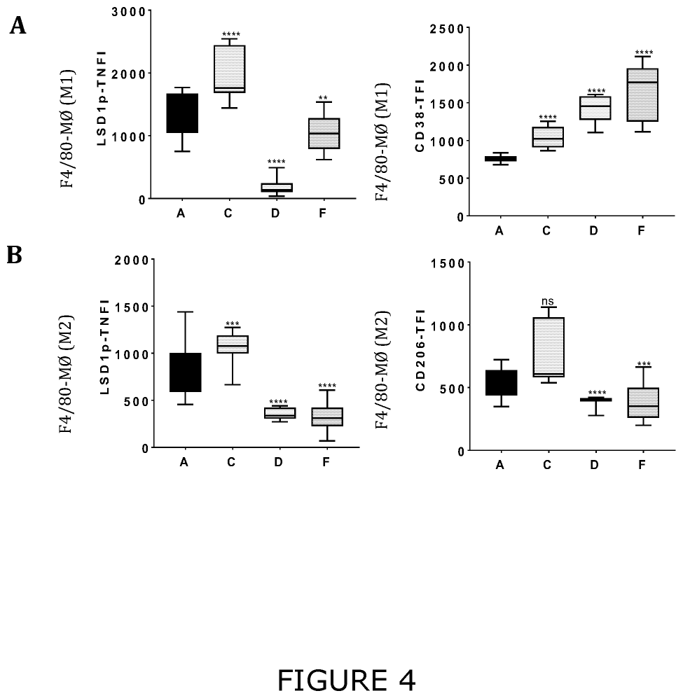

The effect of these treatment modalities was also investigated on TME macrophage populations by immunofluorescence assay (IFA). In particular, the M1 (CD38 expression) and M2 profiles (CD206 expression) were examined, along with LSD1p expression. This analysis found that a tumor associated M2 profile (which is characterized by an increase CD206 and LSD1p expression) is re-programmed and inhibited by phenelzine sulfate but is enhanced by anti-PD1 treatment. The combination treatment was found to strongly inhibit CD206 expression. Of note, the M1 phenotype (CD38 expression and LSD1p inhibition) was most strongly enhanced by phenelzine sulfate alone or by the combination treatment (FIGS. 4A, B).

### Example 3

### Dual Epigenetic-Immunotherapy Re-Educates and Re-Programs Innate and Adaptive Immune Cell Repertoire

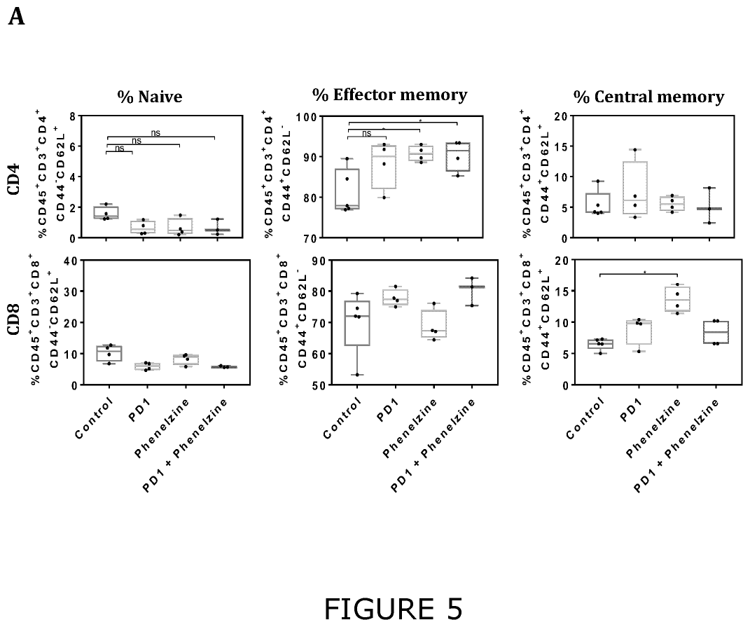

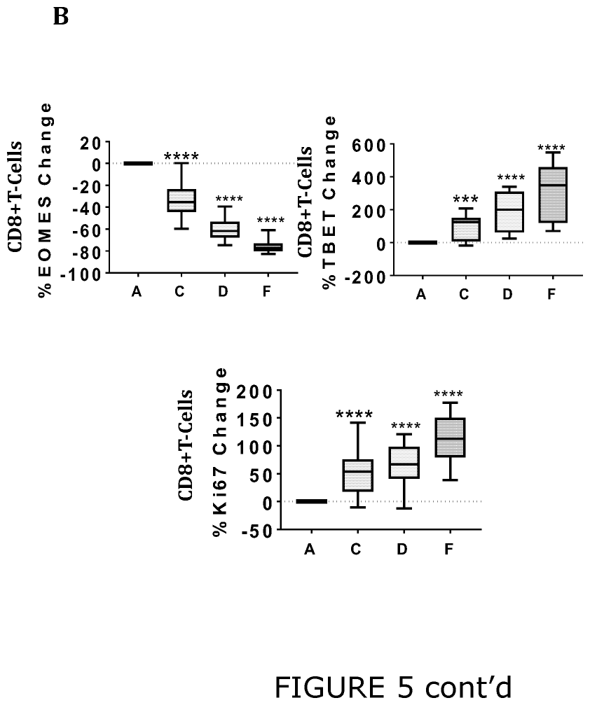

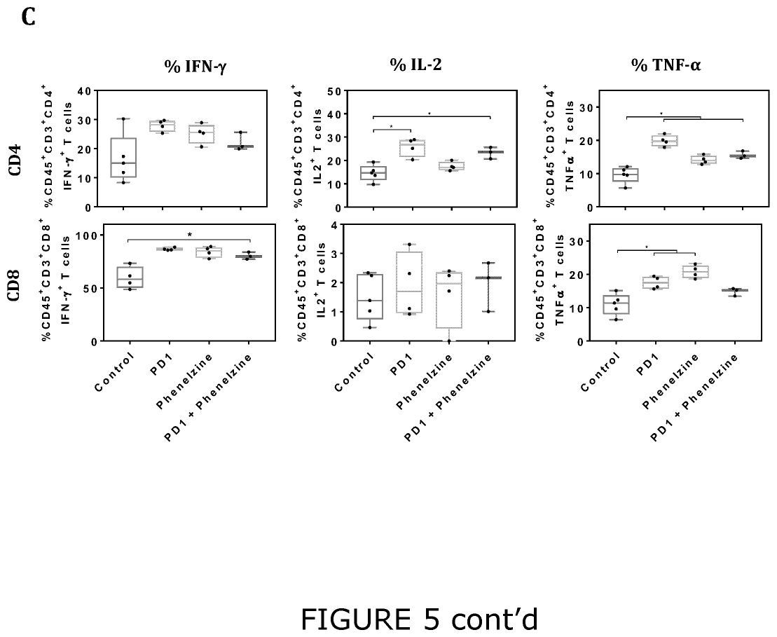

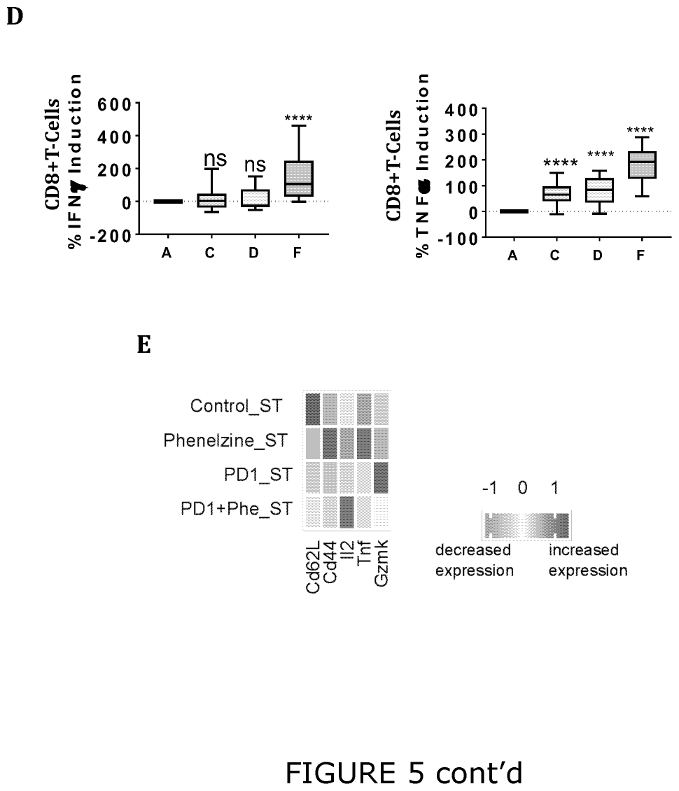

The present inventors also examined the effect of the treatment modalities on the innate and adaptive immune repertoire. They observed some inhibition in the infiltration of CD4/CD8+ naïve T-cells across all 3 treatment groups and increased infiltration of CD4⁺ effector memory T-cells (FIG. 5A). Anti-PD1, phenelzine sulfate and the combination treatments were shown to enhance CD8+ central memory and effector memory populations. (FIG. 5A).

A T-cell exhaustion signature was examined in CD8⁺ T-cells isolated from TME. High expression of EOMES (EOMES^{high}) and low expression of TBET (TBET^{low}) represent an exhaustive T-cell signature in CD8⁺ T-cells. It was found that EOMES, a key marker of exhaustion, was most strongly inhibited by the combination therapy, although anti-PD1 and phenelzine sulfate treatment were also shown to strongly inhibit EOMES expression (FIG. 5B). Consistent with EOMES inhibition, a pattern of induction was observed in TBET and Ki67 expression, which are markers of T-cell activity and effector status. Both these markers were induced by anti-PD1 treatment but to a lesser extent and more strongly by phenelzine sulfate treatment. Notably, the strongest % increase in expression was seen in the combination therapy (FIG. 5B).

In the control group, CD4⁺ and CD8⁺ T-cells were found to be less capable of producing key pro-inflammatory and Th1 cytokines IFN-γ, IL-2 and TNF-α, consistent with an exhaustive signature. By contrast, all treatment groups (i.e., anti-PD1, phenelzine sulfate and combination) were observed to have a better pro-inflammatory/Th1 response when compared to the control, as well as having CD4⁺ T-cells that were much more efficient in producing IL-2 and [symbol] ments also resulted in CD8⁺ T-cells that produce a slightly [symbol] NF-α, but with no significant changes in the production of IL-2. It is also interesting to note that treatment with phenelzine sulfate alone seems to increase the production of these cytokines (FIG. 5C), as compared to the control.

In line with the above FACS analysis, IF analysis of the % change in expression of IFN-γ and TNF-α in CD8⁺ T-cells revealed that anti-PD1 and phenelzine sulfate treatment had no significant effect on IFN-γ expression, whereas strikingly the combination treatment significantly increased expression. In the case of TNF-α % change, there was induction of expression in all 3 treatments with the combination again having the strongest effect (FIG. 5D).

The present inventors also examined the effect of the treatment modalities on the expression of T-cell activation markers in CD8⁺ T cells using the nanostring platform. Compared to control samples, all treatments resulted in decreased expression of Sell (CD62L) and increased CD44 gene expression with more effector memory T cells (CD62L-CD44^{hi}) (FIG. 5E).

Overall, the FACs data indicate that the combination therapy induces a minor but not significant increase in T-cell effector and central memory cell numbers whereas IF protein analysis indicates that these cells express highly significant intensity of effector markers (TBET, Ki67, IFN-γ and TNF-α) and significantly decreased markers for exhaustion (EOMES).

### Example 4

### Nuclear LSD1 Complexes with EOMES in Exhausted T-Cell Signatures

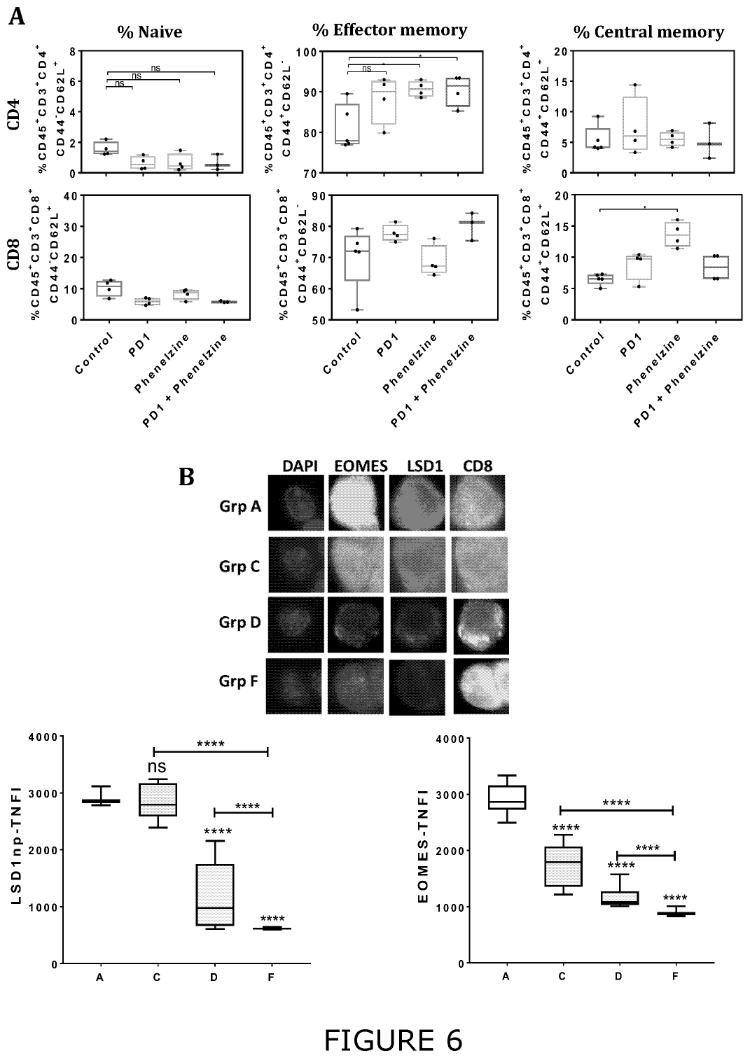

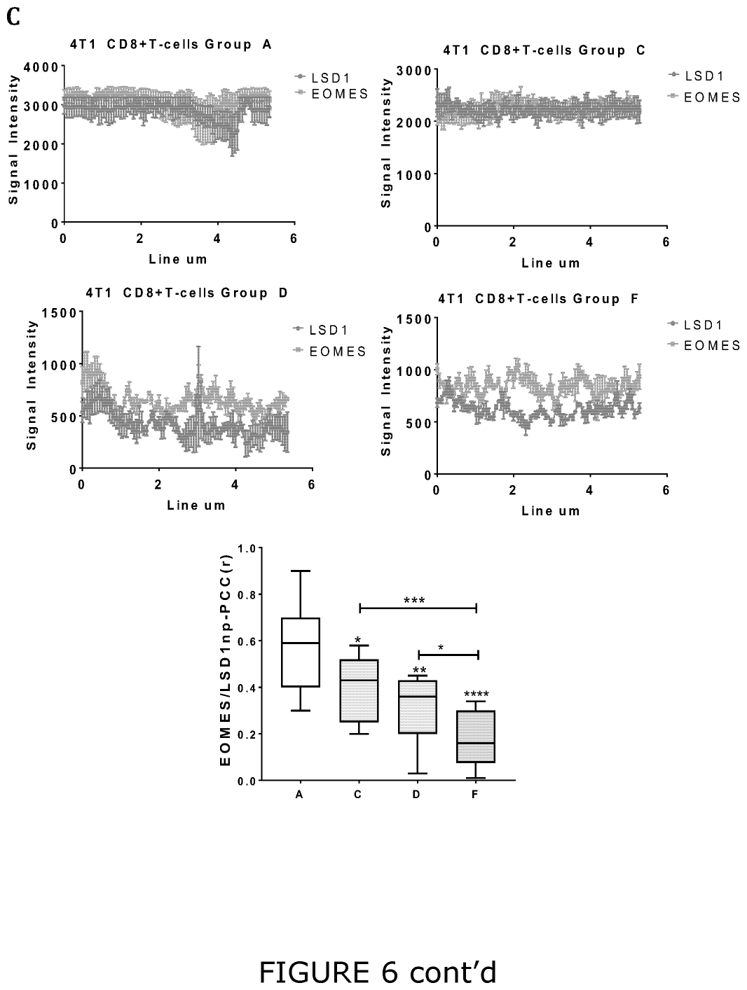

The impact of the treatment modalities was investigated on the T-cell exhaustion signature using the Nanostring platform. Strikingly, all the exhaustion gene profiles demonstrated inhibition when treated with the combination of anti-PD1/phenelzine sulfate therapy with CD39 the most significantly inhibited (FIG. 6A) and CD96 up-regulated.

Interestingly, LSD1 inhibition had minimal impact on the mRNA of T-cell exhaustion genes such as EOMES. By contrast, phenelzine sulfate, which is expected to inhibit the epigenetic activity of LSD1, inhibited exhaustive signature genes such as CTLA4 and LAG3. Like proteins such as P53, LSD1 may be capable of regulating the function of target proteins at both the protein and transcript levels. This regulation would be by post-translational modification of the protein and may well impact nuclear localization and binding partners.

To unravel this role, the present inventors examined the co-expression of LSD1 and EOMES (an exhaustion marker) in CD8⁺ T-cells isolated from the 4T1 metastatic xenograft mouse model. They found that when you treat 4T1 mice with anti-PD1 antibody, EOMES is inhibited but interestingly LSD1 expression is unchanged or slightly increased. However, treatment with either phenelzine sulfate or the combination therapy resulted in marked inhibition of LSD1 and EOMES (FIG. 6B).

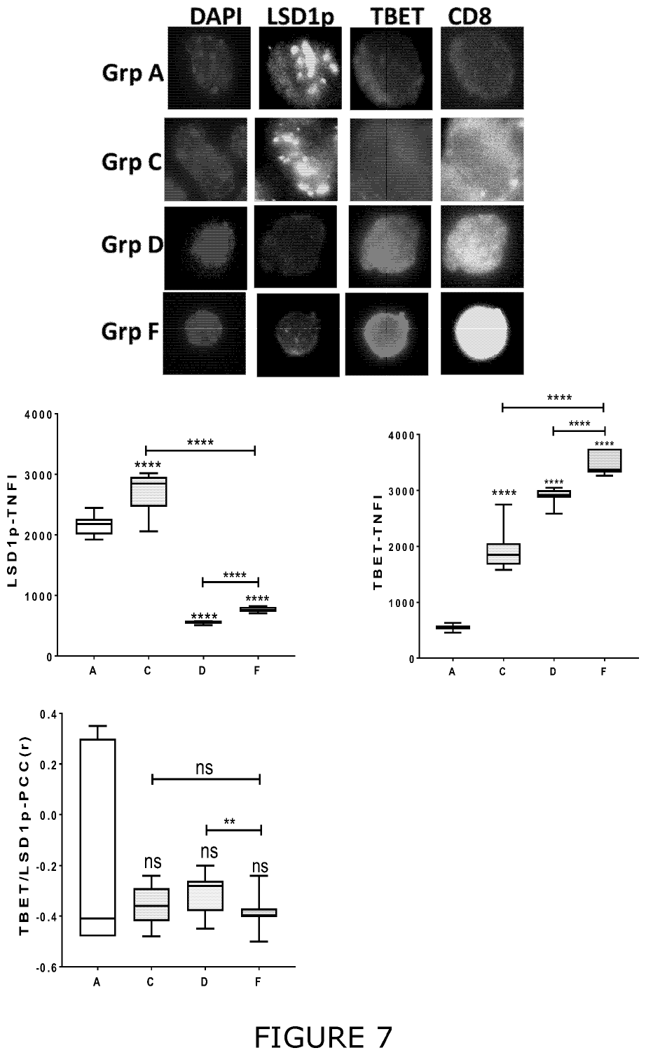

Plot-profiles of EOMES and LSD1 were analyzed in this model as well as partial correlation coefficients (PCC(r)) and it was found that unexpectedly that there is a strong co-localization and relationship between LSD1 and EOMES in the nucleus of exhausted T-cells, suggesting the formation of a nuclear complex between these regulatory proteins. Treatment with phenelzine sulfate or combination immunotherapy markedly inhibited this protein complex which may be implicated in regulating T-cell exhaustion (FIG. 6C). [symbol] ext, the nuclear co-expression of TBET and LSD1p was a [symbol] lls isolated from the 4T1 metastatic xenograft model. An inverse expression relationship was found and in this regard, anti-PD1 antibody increased TBET expression and slightly but significantly increased LSD1p nuclear expression. However, LSD1 inhibition either as a monotherapy or in combination with anti-PD1 immunotherapy significantly inhibited LSD1p expression in the nucleus while simultaneously increasing TBET nuclear expression. The PCC(r) analysis of TBET and LSD1 indicated that these proteins had a negative co-localization and did not occupy the same locations within the nucleus. This negative co-localization was unaffected by treatment with PD1 or LSD1 inhibitors (FIG. 7).

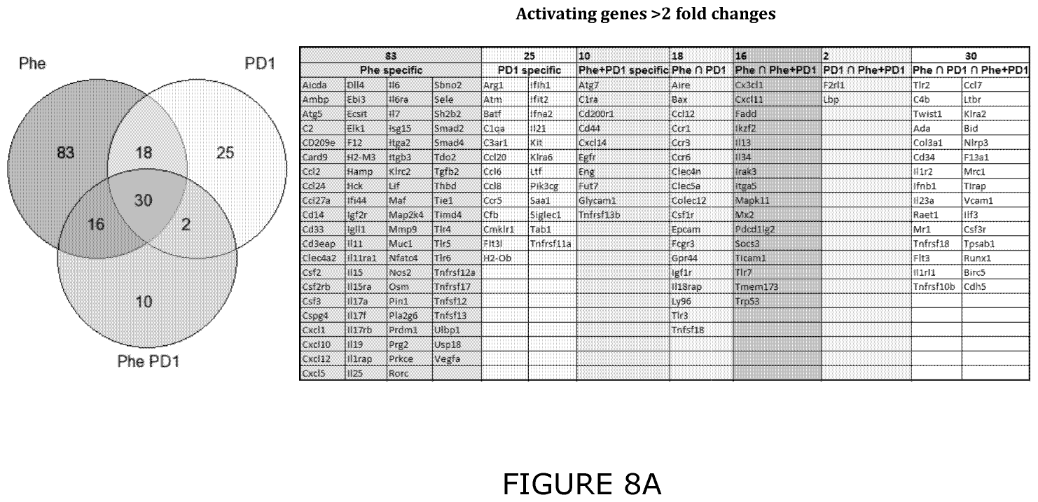

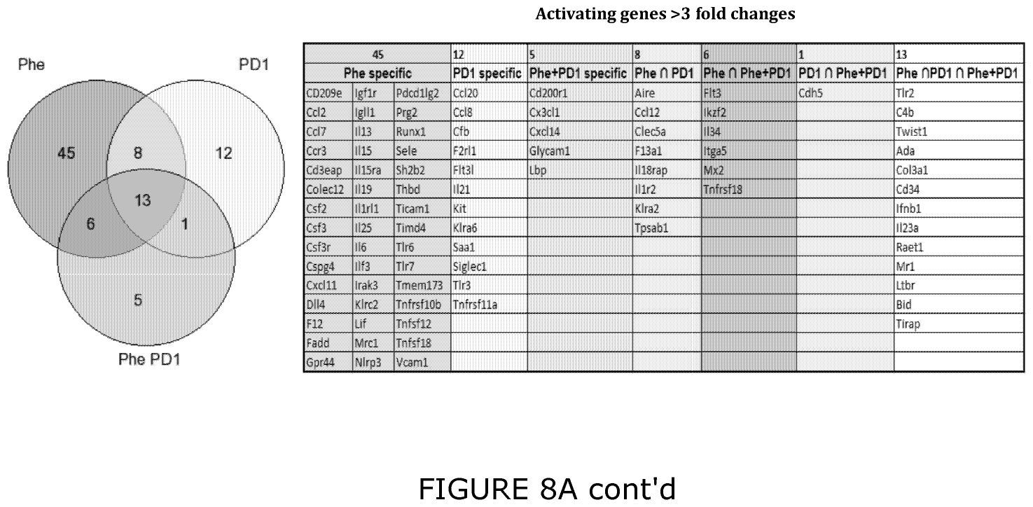

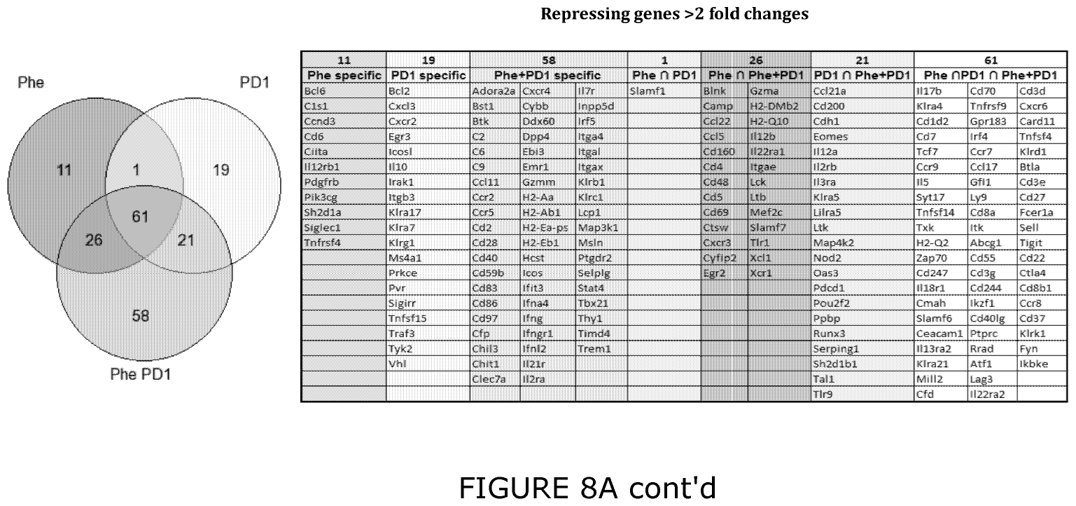

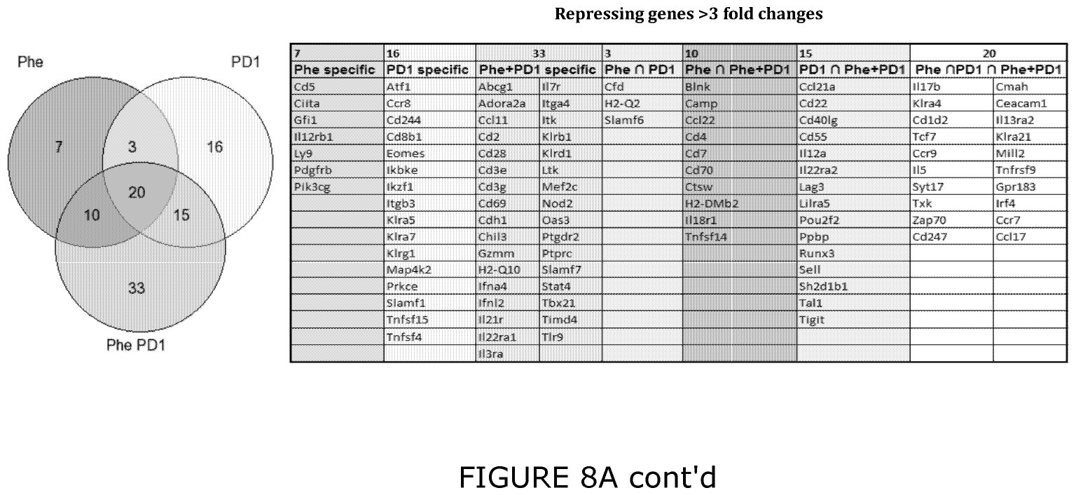

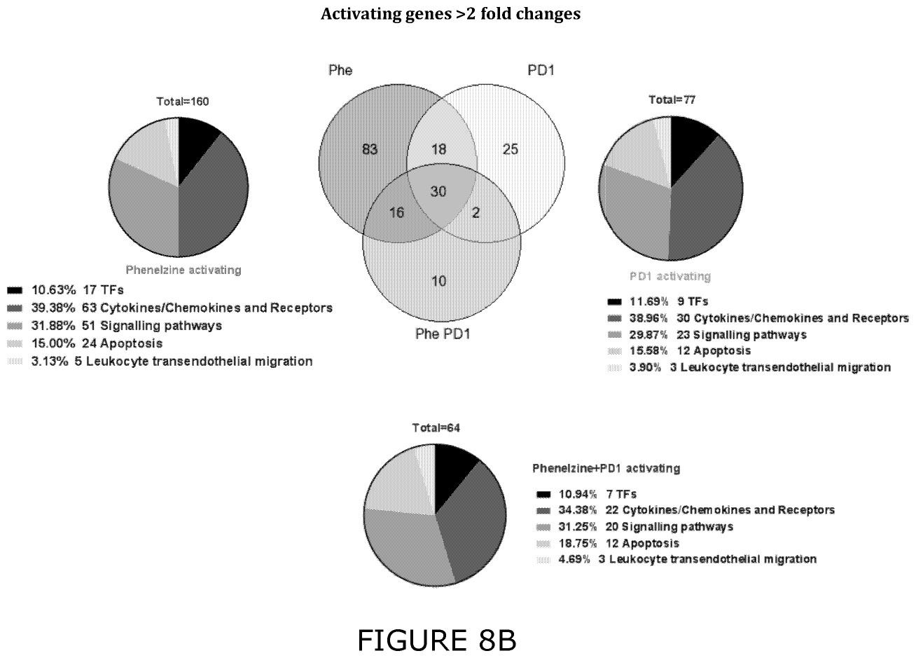

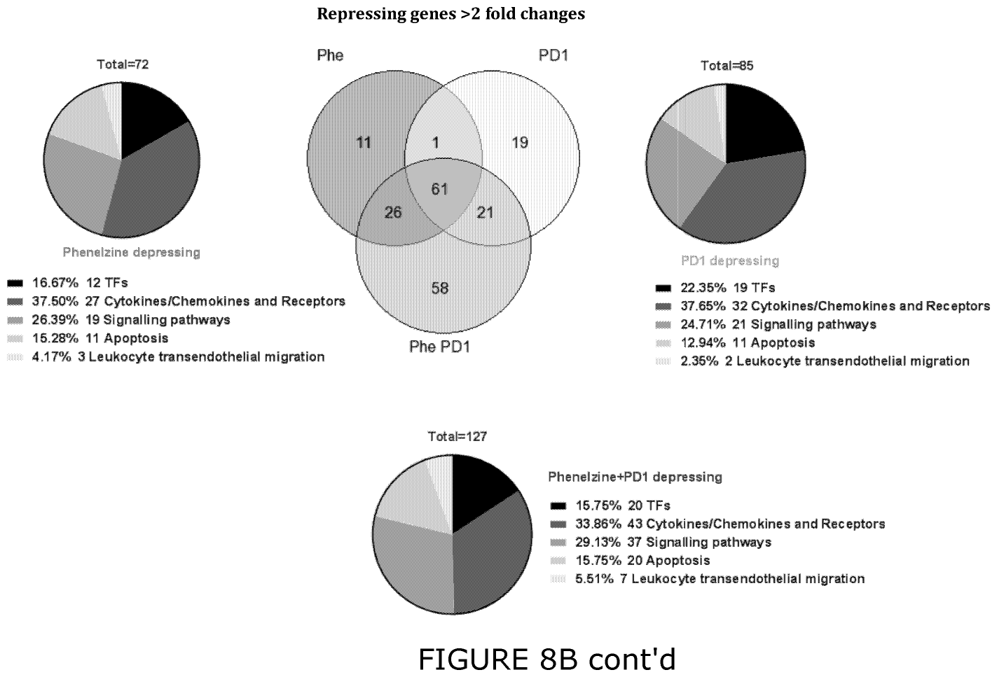

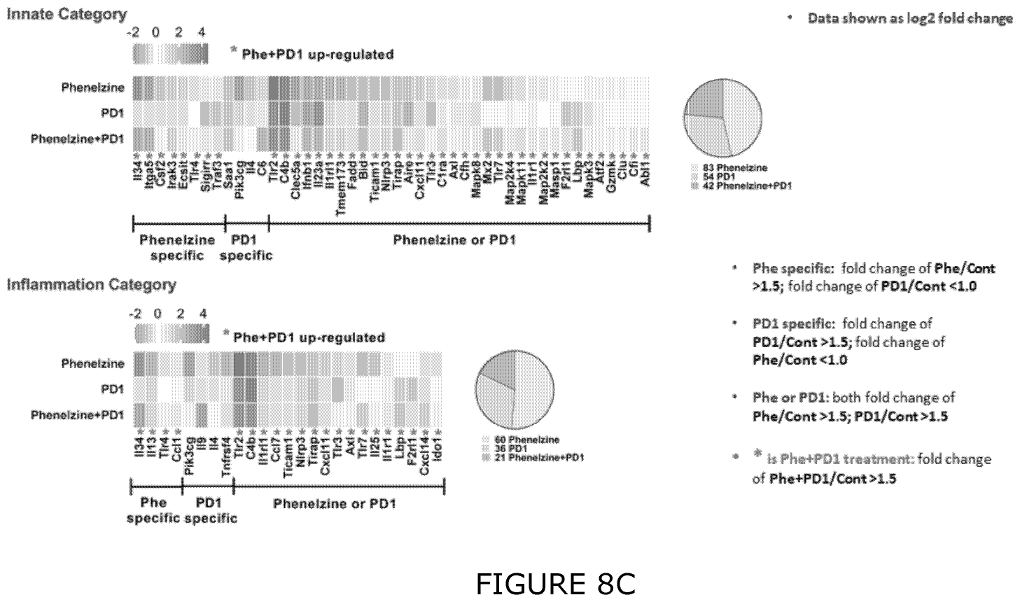

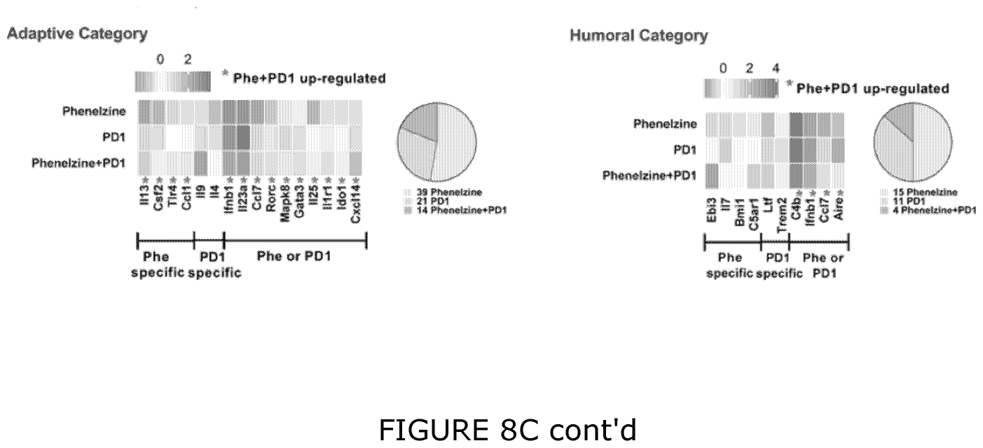

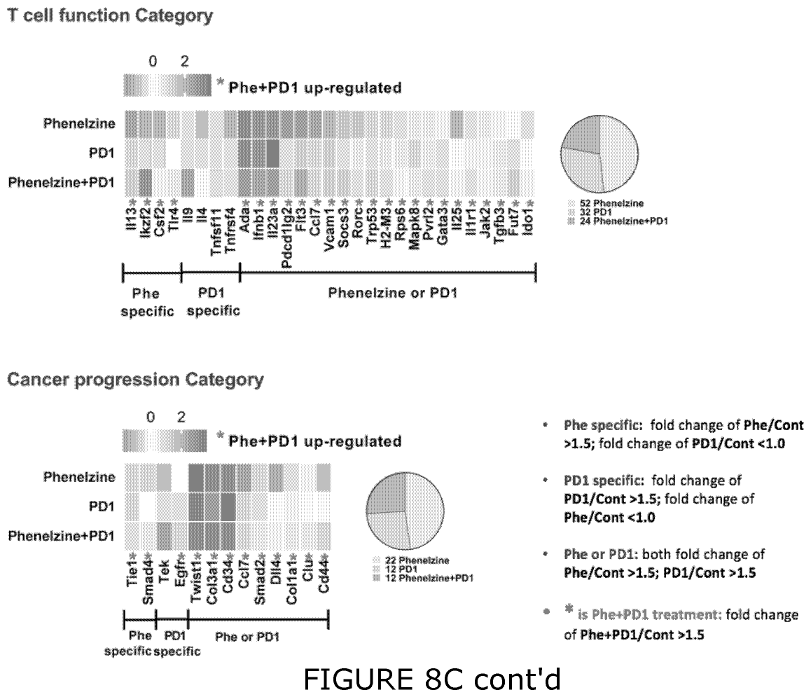

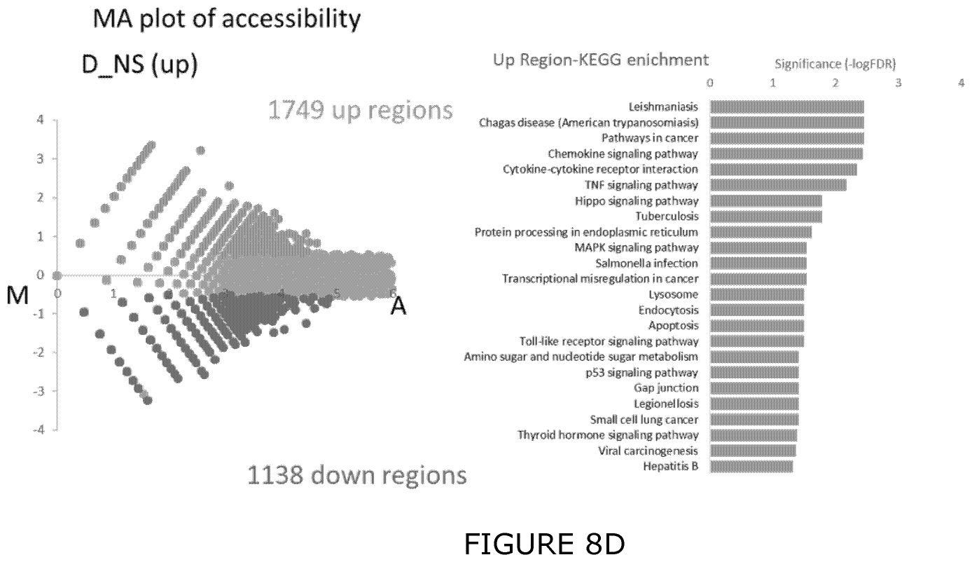

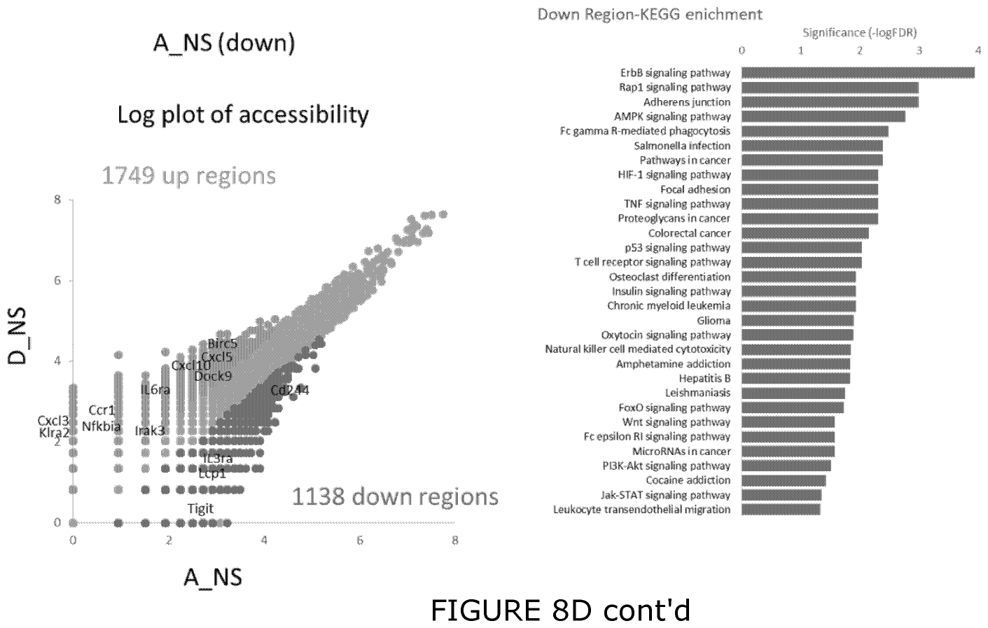

LSD1 inhibition alone or PD1 signaling alone induces or represses distinct gene transcription programs in key signaling pathways (FIG. 8A, B). Importantly, inhibition of LSD1 and PD1 induces and represses gene expression programs involved in adaptive, innate and inflammation signals (FIG. 8C). In this capacity LSD1 re-programs the epigenetic template as confirmed by ATAQ-sequence data, which monitors epigenome changes (FIG. 8D). This re-programming in turn enables the PD1 signals to be received by the epigenetic template resulting in appropriate mRNA production or repression.

### Example 5

### EOMES and LSD1 Form a Complex in Exhausted CD8⁺ T-Cells

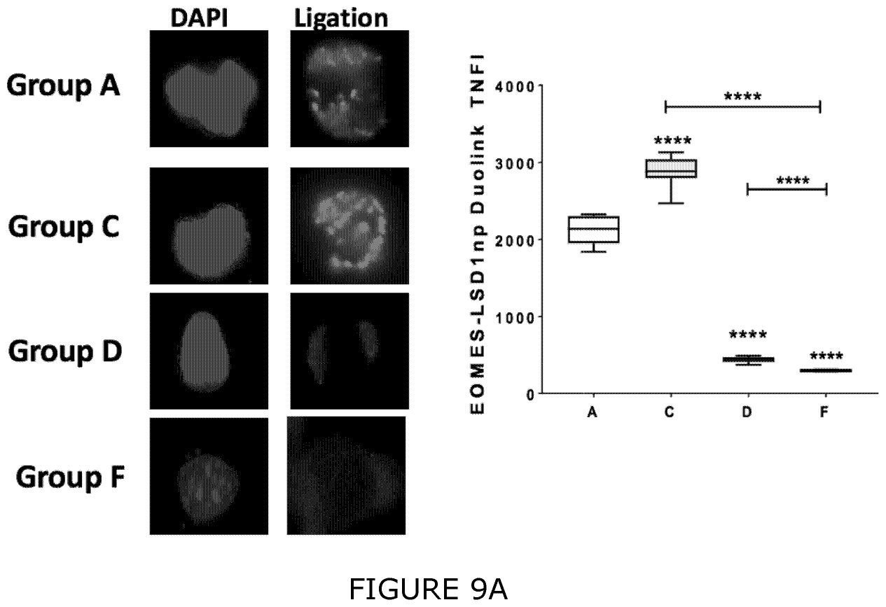

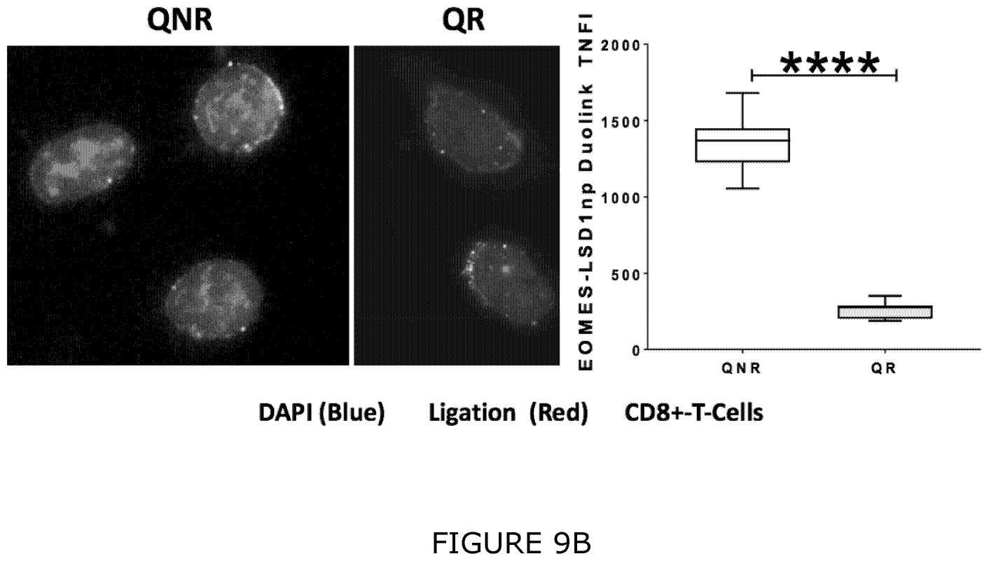

The present inventors sought to investigate the putative complex that LSD1 may form with EOMES though use of the DUOLINK (Sigma) ligation-IF assay, which confirms the presence of interacting proteins via a ligation reaction measured by fluorescent microscopy. Notably, the results of this assay showed a significant positive reaction (FIG. 9A) for this protein complex in Group A (Control) and Group C (PD1 therapy) treated 4T1 mice, clearly indicating that LSD1 and EOMES form a complex in CD8⁺ T-Cells in the TME.

The presence of this complex was also investigated in cytomegalovirus (CMV) reactive (QR) and non-reactive (QNR) CD8⁺ T-cells isolated from patient liquid biopsies. Strikingly, it was found that the QNR samples, which have impaired T-cell function, had significant signal for the EOMES:LSD1 complex (FIG. 9B), strongly suggesting that this complex plays a role in inhibiting T-cell function.

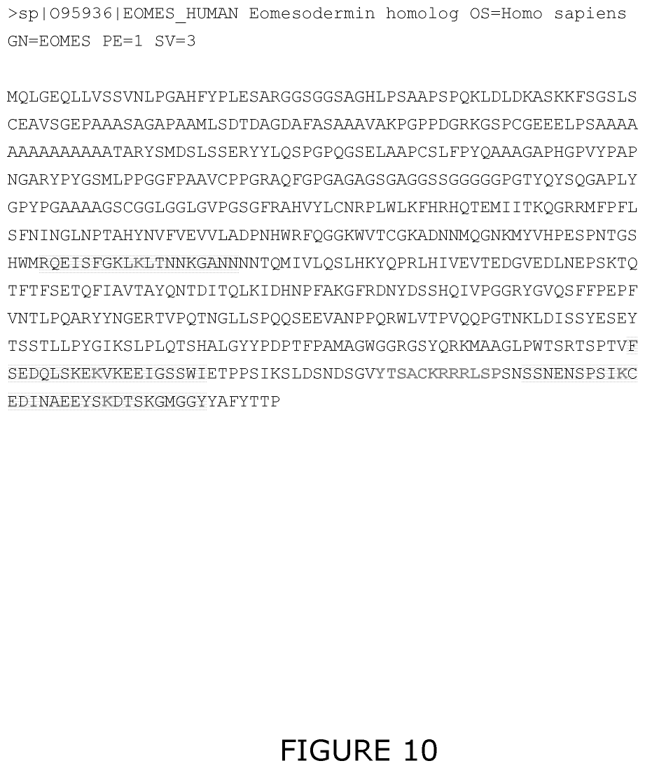

The EOMES protein sequence (FIG. 10) was examined for putative methylation sites and several strongly methylation candidate lysine's were found near the nuclear localization sequence (NLS) of EOMES as well as a strong candidate in the middle of the sequence. Based on the above results, the present inventors predict that LSD1 de-methylates EOMES at one or more of these sites, potentially controlling both protein interaction and nuclear localization.

From the foregoing, there appears to be multilayered regulation by LSD1 in tumor-infiltrating lymphocytes (TILs). This regulation may be indirect or direct. For example, LSD1 may indirectly impact on the epigenome via protein:protein interaction. In this capacity, LSD1 is complexed to EOMES and this is critical to maintain this exhaustive, transcription factor in the nucleus to mediate exhaustive gene signature transcription programs. The present inventors postulate that the interaction between LSD1 with EOMES maintains this transcription factor in a de-methylated state which is crucial for its nuclear retention. On the other hand, LSD1 may directly impact on the epigenome and in this scenario, LSD1 is hypothesized to tether to the epigenetic template and re-program the chromatin structure either in an active or repressor state based on [symbol] e (H3k4me2/h3k9me2). This re-programmed state is beli [symbol] e to PD1 mediated docking of transcription factors and subsequent mRNA expression or repression.

### Example 6

### Triple Therapy Efficacy on CTC/CSC and Tumor Burden

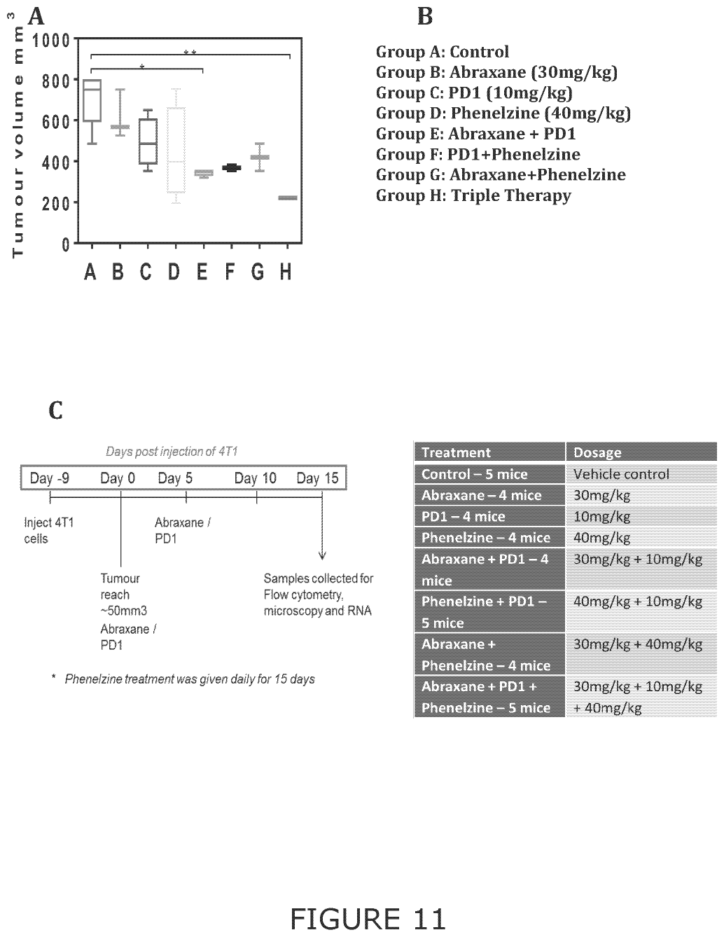

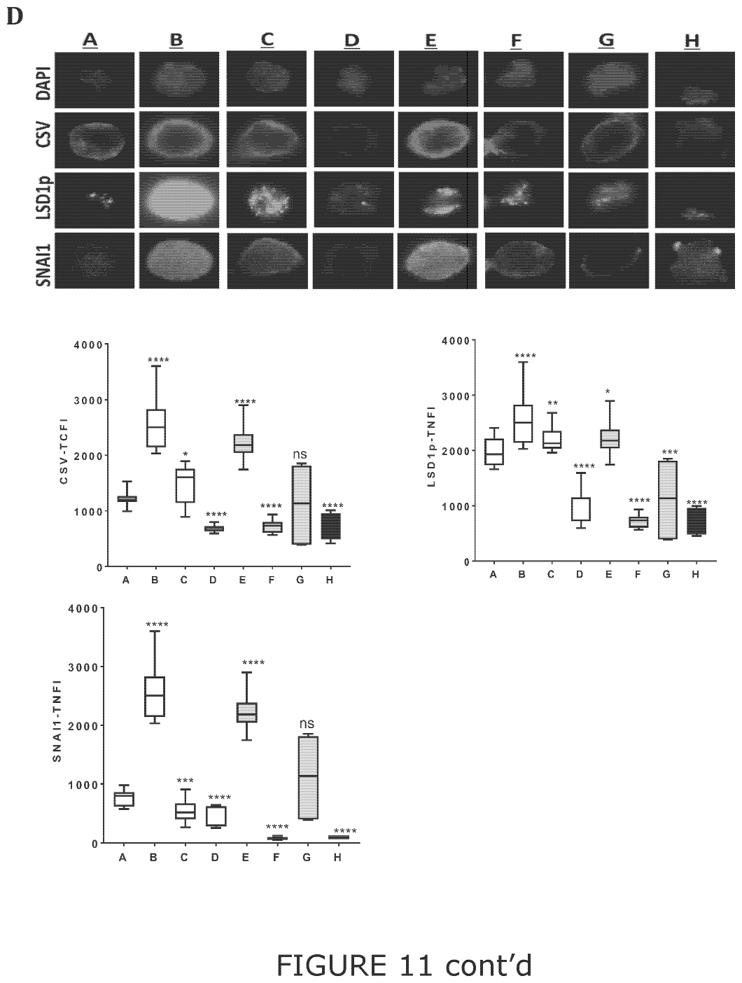

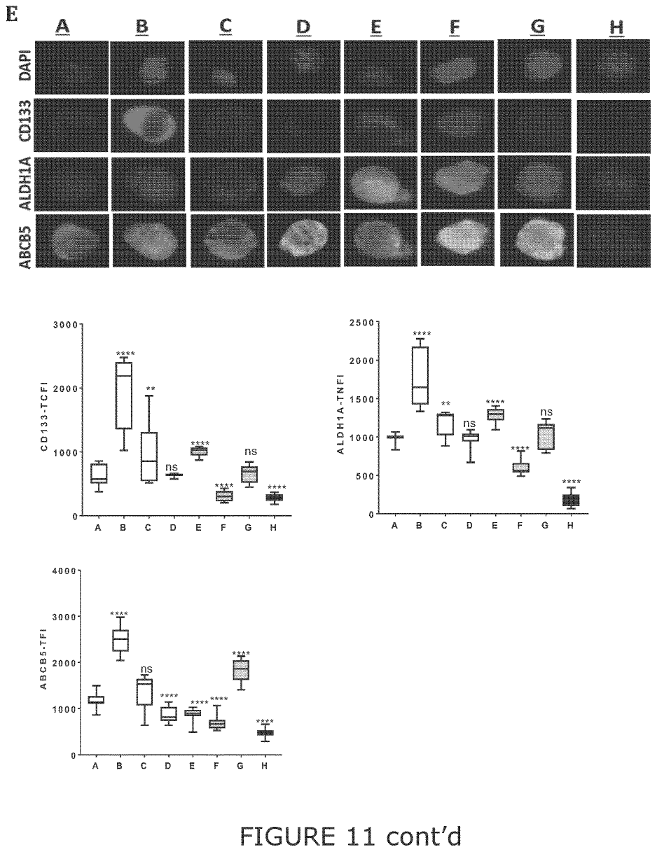

Next, the effect of phenelzine sulfate and the chemotherapeutic drug, Abraxane, was examined in the 4T1 mouse metastatic breast cancer model in the context of PD1 immunotherapy. 4T1 mice were treated with vehicle control (Group A), Abraxane (30 mg/kg) (Group B), anti-PD1 antibody (10 mg/kg) (Group C), phenelzine sulfate (40 mg/kg) (Group D), Abraxane (30 mg/kg)+PD1 antibody (10 mg/kg) (Group E), phenelzine sulfate (40 mg/kg)+PD1 antibody (10 mg/kg) (Group F), Abraxane (30 mg/kg)+phenelzine sulfate (40 mg/kg) (Group G), and Abraxane (30 mg/kg)+phenelzine sulfate (40 mg/kg)+PD1 antibody (10 mg/kg) (also referred to herein as “triple therapy”) (Group H). It was found that all treatments significantly reduced the primary tumor volume (FIG. 11A) with triple therapy providing the most reduction in tumor burden.

Cancer cells harvested from the TME were examined for % change in protein expression relative to vehicle control via IF. The expression of a mesenchymal circulating tumor cell (CTC) signature biomarker panel (CSV, LSD1p, SNAI1) on the cancer cells was assessed and notably, it was found that Abraxane alone increased expression of CSV and SNAI1 and nuclear expression of LSD1 (FIG. 11D, Group B). Additionally, it was found that anti-PD1 immunotherapy alone moderately increased nuclear expression of LSD1, had little effect on expression of CSV and strongly inhibited SNAI1 expression (FIG. 11D, Group C). By contrast, phenelzine sulfate exhibited stronger inhibition on LSD1, CSV and SNAI1 expression (FIG. 11D, Group D) and this inhibition was enhanced in combination with anti-PD1 antibody (FIG. 11D, Group F). The triple therapy overall had the strongest % inhibition of this mesenchymal signature panel (FIG. 11D, Group H).

Next, the cancer cells were assessed for expression of a chemo-resistant, stem-like biomarker signature biomarker panel (CD133, ALDH1A and ABCB5). The % change in expression in the Abraxane treated group showed a marked increase in expression of CD133, ALDH1A and ABCB5 (FIG. 11E, Group C). The % change in expression in the anti-PD1 treated group showed a moderate increase in expression of ALDH1A and CD133 but no change in expression of ABCB5 (FIG. 11E, Group C). Additionally, phenelzine sulfate alone did not significantly affect expression of CD133 and ALDHA1 but strongly inhibited ABCB5 expression (FIG. 2C, Group D). Strikingly, the phenelzine sulfate+anti-PD1 dual therapy significantly inhibited all 3 biomarkers of this stem-like, chemo-resistant signature panel (FIG. 11E, Group F) with the triple therapy combination providing the strongest inhibition (FIG. 11E, Group H).

### Materials and Methods

4T1 Mouse Model and Microscopy Methods

A total of 2×10⁵ cells were injected per mouse into mammary gland in 50 μL of phosphate buffered saline (PBS). Treatments were started on mice after 15 days post-inoculation of 4T1 cells. Treatment groups are as follows: Group A=Control, Group C: PD1 (10 mg/kg), Group D: Phenelzine (40 mg/kg), Group F: PD1+Phenelzine. PD1 treatments were given every 5 days. Tumors were measured using calipers and the tumor volumes (mm3) were calculated using the formula (length×width2)/2. [symbol] arvested cells from primary tumors where then cytospun [symbol] -treated with poly-1-lysine and fixed then stored in PBS for IFA Microscopy Analysis. Cells were permeabilized by incubating with 1% Triton X-100 for 20 min and were probed with a variety of primary antibodies as described in the figure legends and the corresponding secondary antibodies. Cover slips were mounted on glass microscope slides with ProLong Diamond Antifade reagent (Life Technologies). Protein targets were localized by confocal laser scanning microscopy. Single 0.5 μm sections were obtained using a Leica DMI8 microscope using 100× oil immersion lens running LAX software. The final image was obtained by averaging four sequential images of the same section. Digital images were analysed using ImageJ software (ImageJ, NIH, Bethesda, Md., USA) to determine the either the Total Nuclear Fluorescent Intensity (TNFI), the Total Cytoplasmic Fluorescent Intensity (TCFI) or total Fluorescent Intensity (TFI). Graph represents the TNFI values, TCFI or TFI for each cell measured using ImageJ to select the nucleus minus background (n>20 individual cells).

4T1 FFPE Analysis

4T1 Treatment FFPE from each treatment group primary tumor biopsies were processed in the BondRX for IFA staining using the instrument protocol: ER2 for 20 mins at 100° C. with Epitope Retrieval Solution 2 which is a pH-9 EDTA based retrieval solution followed by probing with rabbit anti LSD1 (S111p); mouse anti CSV and goat anti ALDH1A and visualized with a donkey anti-rabbit AF 488, anti-mouse 568 and anti-goat 633 or anti rat 633. Cover slips were mounted on glass microscope slides with ProLong Diamond Antifade reagent (Life Technologies). Protein targets were localized by confocal laser scanning microscopy. Single 0.5 μm sections were obtained using a Leica DMI8 microscope using 100× oil immersion lens running LAX software. The final image was obtained by averaging four sequential images of the same section. Digital images were analysed using ImageJ software (ImageJ, NIH, Bethesda, Md., USA) to determine the either the Total Nuclear Fluorescent Intensity (TNFI), the Total Cytoplasmic Fluorescent Intensity (TCFI) or total Fluorescent Intensity (TFI).

FACS Analysis

Cells were surfaced stained with CD49b, F4/80 and intracellular stained for IFN-γ, TNF-α and IL-10. To label NK cells and the macrophages (M1/M2) in the tumor microenvironment or a suite of antibodies to label CD8⁺ T-Cells (Cells were surfaced stained with CD45, CD3, CD4, CD8, CD44, CD62L (for naïve, effector and central memory). Cells were finally resuspended in PBS 2% FBS and flow cytometry performed on FACS Fortessa (BD) or FACS LSRII (BD). Analysis of data was done using FlowJo® analysis software and % cell populations calculated from the raw data. A Mann-Whitney non-parametric t-test was used to compare control vs other groups.

Nanostring Methods

CD8⁺ T-cells were isolated from the 4T1 metastatic mouse model with high purity using the StemCell technologies CD8 isolation Kit. Qiagen mRNA prep kit was used to generate mRNA and then processed for nanostring analysis using manufactures guidelines and protocols and profiled with the immuno-oncology gene panel.

DUO-Link Analysis

The DUO-Link ligation was employed to measure the co-interaction of two proteins of interest (EOMES and LSD1np) via a ligation/amplification IFA as per the manufactures protocol and SOP. Analysis was carried out measuring the intensity of the corresponding IF which [symbol] scopy with positive signals corresponding to a successful [symbol] the target two target proteins interacting.

QR and QNR CMV Patient Samples

QR patient group included immune reactive (R) HSCT recipients who acquired stable anti-CMV T cell immunity as indicated by QuantiFERON-CMV reactivity (≥0.1 IU/mL) and no evidence of viral recrudescence. The QNR group included immune non-reactive (NR), HSCT recipients who failed to acquire stable anti-CMV T cell immunity as indicated by QuantiFERON-CMV reactivity (<0.1 IU/mL) and with symptomatic viral recrudescence (single or multiple viral reactivations) or asymptomatic viral recrudescence. The QuantiFERON-CMV assay (QIAGEN, Hilden, Germany) measures the amount of CMV-specific IFN-γ secretion in whole blood

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A composition for enhancing T-cell (e.g., CD8⁺ T-cell or CD4⁺ T-cell) function, or for treating a T-cell dysfunctional disorder, the composition comprising, consisting or consisting essentially of a LSD inhibitor and a PD-1 binding antagonist.

2. The composition of claim 1, wherein the LSD inhibitor is an inhibitor of LSD enzymatic activity.

3. The composition of claim 2, wherein the LSD inhibitor is a monoamine oxidase (MAO) inhibitor.

4. The composition of claim 3, wherein the MAO inhibitor is selected from: clorgyline; L-deprenyl; isocarboxazid (Marplan™); ayahuasca; nialamide; iproniazide; iproclozide; moclobemide (Aurorix™; 4-chloro-N-(2-morpholin-4-ylethyl)benzamide); phenelzine (Nardil™; (±)-2-phenylethylhydrazine); tranylcypromine (Parnate™; (±)-trans-2-phenylcyclopropan-1-amine) (the congeneric of phenelzine); toloxatone; levo-deprenyl (Selegiline™); harmala; RIMAs (e.g., moclobemide, described in Da Prada et al. (1989. J Pharmacol Exp Ther 248:400-414); brofaromine; and befloxatone, described in Curet et al. (1998. J Affect Disord 51: 287-30), lazabemide (Ro 19 6327), described in Ann. Neurol., 40(1): 99-107 (1996), and SL25.1131, described in Aubin et al. (2004. J. Pharmacol. Exp. Ther. 310: 1171-1182); selegiline hydrochloride (1-deprenyl, ELDEPRYL, ZELAPAR); dimethylselegilene; safinamide; rasagiline (AZILECT); bifemelane; desoxypeganine; harmine (also known as telepathine or banasterine); linezolid (ZYVOX, ZYVOXID); pargyline (EUDATIN, SUPIRDYL); dienolide kavapyrone desmethoxyyangonin; and 5-(4-Arylmethoxyphenyl)-2-(2-cyanoethyl)tetrazoles.

5. The composition of claim 2, wherein the LSD inhibitor is a compound represented by formula (XII): 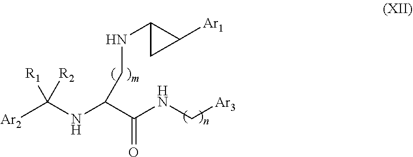
wherein:
Ar₁ is a 5 to 7 membered aryl or heteroaryl ring;
Ar₂ and Ar₃ are each independently selected from a 5 to 7 membered aryl or heteroaryl ring, optionally substituted with 1 to 3 substituents;
R₁ and R₂ are independently selected from hydrogen and hydroxyl or taken together R₁ and R₂ form ═O, ═S or ═NR₃;
R₃ is selected from hydrogen, —C₁₋₆ alkyl or —OH;
m is an integer from 1 to 5; and
n is an integer from 1 to 3;
or a pharmaceutically acceptable salt thereof.

6. The composition of claim 5, wherein one or more of the following applies:
Ar₁ is a six membered aryl or heteroaryl ring, especially phenyl, pyridine, pyrimidine, pyrazine 1,3,5-triazine, 1,2,4-trazine and 1,2,3-triazine, more especially phenyl;
Ar₂ is a six membered aryl or heteroaryl ring, especially phenyl, pyridine, pyrimidine, pyrazine 1,3,5-triazine, 1,2,4-trazine and 1,2,3-triazine, especially phenyl; especially where the six membered aryl or heteroaryl ring is optionally substituted with one optional substituent, especially in the 3 or 4 position;
Ar₃ is a six membered aryl or heteroaryl ring, especially phenyl, pyridine, pyrimidine, pyrazine 1,3,5-triazine, 1,2,4-trazine and 1,2,3-triazine, especially phenyl; especially where the six membered aryl or heteroaryl ring is optionally substituted with one optional substituent, especially in the 3 or 4 position.

7. The composition of claim 5 or claim 6, wherein
optional substituents for Ar₁ and Ar₂ include —C₁₋₆alkyl, —C₂₋₆alkenyl, —CH₂F, —CHF₂, —CF₃, halo, aryl, heteroaryl, —C(O)NHC₁₋₆alkyl, —C(O)NHC₁₋₆alkylNH₂, —C(O)— heterocyclyl, especially methyl, ethyl, propyl, butyl, t-butyl, —CH₂F, —CHF₂, —CH₃, Cl, F, phenyl, —C(O)NH(CH₂)₁₋₄NH₂ and —C(O)-heterocyclyl;
R₁ and R₂ taken together form ═O, ═S or ═NR₃, especially ═O or ═S, more especially ═O;
R₃ is H, —C₁₋₃alkyl or —OH, especially H, —CH₃ or —OH.
m is 2 to 5, especially 3 to 5, more especially 4,
n is 1 or 2, especially 1.

8. The composition of any one of claims 5 to 7, wherein the LSD inhibitor is a compound represented by formula (XIIa): 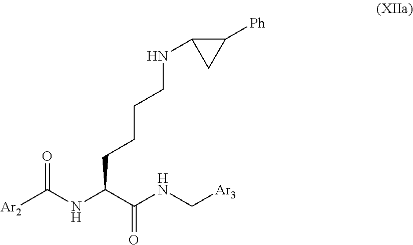
wherein:
Ar₂ and Ar₃ are each independently selected from a 5 to 7 membered aryl or heteroaryl ring, optionally substituted with 1 to 3 substituents.

9. The composition of any one of claims 5 to 8, wherein Ar₂ and Ar₃ are selected as follows:

| Compound | Ar₂ | Ar₃ |
|---|---|---|
| 1b | phenyl | phenyl |
| 1c | 4-nnethylphenyl | phenyl |
| 1d | 4-t-butylphenyl | phenyl |
| 1e | 4-chlorophenyl | phenyl |
| 1f | 4-fluorophenyl | phenyl |
| 1g | 4-phenyl-phenyl | Phenyl |
| 1h | 4-trifluoromethylphenyl | Phenyl |
| 1i | 3-(2-aminoethylcarbamoyl)phenyl | Phenyl |
| 1j | 3-(piperazine-l-carbonyl)phenyl | Phenyl |
| 1k | 4-phenyl-phenyl | 4-methylphenyl |
| 1l | 4-phenyl-phenyl | 4-fluorophenyl |
| 1m | 4-phenyl-phenyl | 4-phenyl-phenyl |
| 1n | 4-phenyl-phenyl | 4-t-butylphenyl |
| 1o | 4-phenyl-phenyl | 3-methylphenyl |
| 1p | 4-phenyl-phenyl | 3-fluorophenyl |
| 1q | 4-phenyl-phenyl | 3-phenyl-phenyl |

10. The composition of any one of claims 5 to 9, wherein the LSD inhibitor is a compound represented by the following structure:

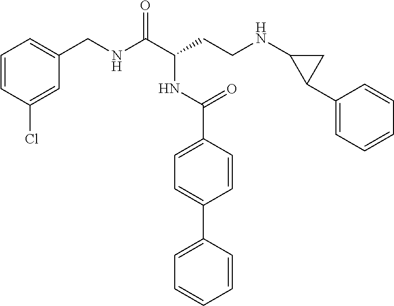 [SMILES: O=C(N[C@@H](CCNC1CC1c1ccccc1)C(=O)NCc1cccc(Cl)c1)c1ccc(-c2ccccc2)cc1]

11. The composition of claim 1, wherein the LSD inhibitor is an inhibitor of LSD nuclear translocation.

12. The composition of claim 11, wherein the LSD inhibitor is a peptide corresponding to the nuclear localization site of LSD.

13. The composition of claim 11, wherein the LSD inhibitor is an isolated or purified proteinaceous molecule represented by formula XIX:
Z₁RRTX₁RRKRAKVZ₂ (XIX)
wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof.

14. The composition of claim 13, wherein “X₁” is selected from S and A.

15. The composition of claim 13 or claim 14, wherein “Z₁” is a proteinaceous molecule represented by formula XX:
X₂X₃X₄ (XX)
wherein:
“X₂” is absent or is a protecting moiety;
“X₃” is absent or is selected from any amino acid residue; and
“X₄” is selected from any amino acid residue.

16. The composition of claim 15, wherein “X₃” is selected from basic amino acid residues including R, K and modified forms thereof.

17. The composition of claim 15 or claim 16, wherein “X₄” is selected from aromatic amino acid residues, including F, Y, W and modified forms thereof.

18. The composition of any one of claims 13 to 17, wherein “Z₂” is absent.

19. The composition of any one of claims 13 to 18, wherein the isolated or purified proteinaceous molecule of formula XIX comprises, consists or consists essentially of an amino acid sequence represented by SEQ ID NO: 2, 3 or 4:
[SEQ ID NO: 1]
RRTSRRKRAKV;
[SEQ ID NO: 2]
RRTARRKRAKV;
or
[SEQ ID NO: 3]
RWRRTARRKRAKV.

20. The composition of any one of claims 13 to 19, wherein the proteinaceous molecule of formula XIX further comprises at least one membrane permeating moiety.

21. The composition of claim 20, wherein the membrane permeating moiety is a lipid moiety.

22. The composition of claim 20 or claim 21, wherein the membrane permeating moiety is a myristoyl group.

23. The composition of any one of claims 20 to 22, wherein the membrane permeating moiety is conjugated to the N- or C-terminal amino acid residue of the proteinaceous molecule of formula XIX.

24. The composition of any one of claims 1 to 23, wherein the PD-1 binding antagonist inhibits the binding of PD-1 to PD-L1 and/or PD-L2.

25. The composition of any one of claims 1 to 24, wherein the PD-1 binding antagonist is an anti-PD-1 antagonist antibody.

26. The composition of claim 25, wherein the anti-PD-1 antagonist antibody is selected from nivolumab, pembrolizumab, lambrolizumab and pidilizumab.

27. The composition of any one of claims 1 to 24, wherein the PD-1 binding antagonist is an immunoadhesin (e.g., AMP-224).

28. The composition of any one of claims 1 to 27, further comprising an ancillary agent for treating, or for aiding in the treatment of, a T-cell dysfunctional disorder.

29. The composition of claim 28, wherein the ancillary agent is a chemotherapeutic agent.

30. The composition of claim 29, wherein the chemotherapeutic agent is an agent that targets rapidly dividing cells and/or disrupt the cell cycle or cell division.

31. The composition of claim 29 or claim 30, wherein the chemotherapeutic agent is a cytotoxic agent.

32. The composition of claim 31, wherein the cytotoxic agent is taxane.

33. The composition of claim 32, wherein the taxane is paclitaxel.

34. The composition of claim 32, wherein the taxane is Abraxane.

35. The composition of any one of claims 1 to 34, further comprising a pharmaceutically acceptable carrier.

36. A method of enhancing T-cell function, the method comprising, consisting or consisting essentially of contacting a T-cell with a LSD inhibitor and a PD-1 binding antagonist, to thereby enhance T-cell function.

37. The method of claim 31, wherein the enhanced T-cell function includes any one or more of elevated biomarkers of T-cell activation and effector capacity (e.g., IFN-γ, TNF-α, Ki67 and TBET), increased proliferation of T-cells, including effector T-cells and/or memory T-cells, increased activation of T-cells including CD4⁺ and CD8⁺ T-cells, increased recognition of an antigen or an antigen peptide derived from an antigen in the context of MHC class II molecules by T-cell receptors, increased recognition of an antigen or an antigen peptide derived from an antigen in the context of MHC class I molecules by T-cell receptors, increased elimination of cells presented in the context of MHC class I molecules and increased cytolytic killing of antigen expressing target cells. In some embodiments, the T-cell has a mesenchymal phenotype.

38. The method of claim 36 or claim 37, wherein the T-cell has a mesenchymal phenotype.

39. The method of any one of claims 36 to 38, wherein the T-cell has aberrant expression of nuclear LSD.

40. The method of claim 39, wherein the T-cell expresses nuclear LSD at a higher level than the level of expression of TBET in the same T-cell, and/or at a higher level than in an activated T-cell.

41. The method of any one of claims 36 to 40, wherein the T-cell is one exhibiting T-cell exhaustion or anergy.

42. The method of claim 41, wherein the T-cell expresses a higher level of EOMES than TBET and/or has elevated expression of PD-1.

43. The method of any one of claims 36 to 42, wherein the T-cell is a CD8⁺ T-cell.

44. A method of enhancing immune effector function of an immune effector cell that expresses PD-1, the method comprising, consisting or consisting essentially of contacting the immune effector cell with a LSD inhibitor and a PD-1 binding antagonist, to thereby enhance the immune effector function of the immune effector cell.

45. The method of claim 44, wherein the enhanced immune effector function includes any one or more of increased recognition of an antigen or an antigen peptide derived from an antigen in the context of MHC class II molecules by T-cell receptors, increased release of cytokines and/or the activation of CD4⁺ lymphocytes, increased release of cytokines and/or the activation of CD8⁺ lymphocytes (CTLs) and/or B-cells, increased recognition of an antigen or an antigen peptide derived from an antigen in the context of MHC class I molecules by T-cell receptors, increased elimination of cells presented in the context of MHC class I molecules, i.e., cells characterized by presentation of an antigen with class I MHC, for example, via apoptosis or perforin-mediated cell lysis, increased production of cytokines such as IFN-γ and TNF-α, and increased specific cytolytic killing of antigen expressing target cells.

46. The method of claim 45, wherein the immune effector has aberrant expression of nuclear LSD.

47. The method of claim 45 or claim 46, wherein the immune effector expresses nuclear LSD at a higher level than the level than in a control immune effector cell (e.g., an immune effector cells with normal or non-repressed immune effector function).

48. A method of treating a T-cell dysfunctional disorder in a subject, the method comprising, consisting or consisting essentially of administering concurrently to the subject a LSD inhibitor and a PD-1 binding antagonist in effective amounts to treat the T-cell dysfunctional disorder.

49. The method of claim 48, wherein the LSD inhibitor and PD-1 binding antagonist are administered in synergistically effective amounts.

50. The method of claim 48 or claim 49, wherein the T-cell dysfunctional disorder is a disorder or condition of T-cells characterized by decreased responsiveness to antigenic stimulation and/or increased inhibitory signal transduction through PD-1.

51. The method of any one of claims 48 to 50, wherein the T-cell dysfunctional disorder is one in which the T-cells have decreased ability to secrete cytokines, proliferate, or execute cytolytic activity.

52. The method of any one of claims 48 to 51, wherein the decreased responsiveness to antigenic stimulation results in ineffective control of a pathogen or tumor.

53. method of any one of claims 48 to 52, wherein the T-cell dysfunctional disorder is one in which T-cells are anergic.

54. The method of any one of claims 48 to 53, wherein the T-cell dysfunctional disorders is selected from unresolved acute infection, chronic infection and tumor immunity.

55. The method of any one of claims 48 to 54, wherein the T-cell dysfunctional disorder is a cancer or infection that comprises a T-cell (e.g., a CD8⁺ T-cell or CD4⁺ T-cell) with a mesenchymal phenotype.

56. The method of any one of claims 48 to 55, wherein the T-cell expresses nuclear LSD at a higher level than the level of expression of TBET in the same T-cell, and/or at a higher level than in an activated T-cell.

57. The method of any one of claims 48 to 56, wherein the T-cell is one exhibiting T-cell exhaustion or anergy.

58. The method of any one of claims 48 to 57, wherein the T-cell expresses a higher level of EOMES than TBET and/or has elevated expression of PD-1.

59. The method of any one of claims 48 to 58, wherein the T-cell is a tumor-infiltrating lymphocyte.

60. The method of any one of claims 48 to 59, wherein the T-cell is a circulating lymphocyte.

61. The method of any one of claims 48 to 60, wherein the cancer is skin cancer (e.g., melanoma), lung cancer, breast cancer, ovarian cancer, gastric cancer, bladder cancer, pancreatic cancer, endometrial cancer, colon cancer, kidney cancer, esophageal cancer, prostate cancer, colorectal cancer, glioblastoma, neuroblastoma, or hepatocellular carcinoma.

62. The method of claim 61, wherein the cancer is a metastatic cancer.

63. The method of claim 62, wherein the metastatic cancer is metastatic breast cancer, metastatic liver cancer or metastatic lung cancer.

64. The method of any one of claims 48 to 63, further comprising administering concurrently to the subject, with the LSD inhibitor and the PD-1 binding antagonist, an ancillary agent (e.g., a chemotherapeutic agent) or ancillary therapy (e.g., ablation or cytotoxic therapy) for treating, or for aiding in the treatment of, a T-cell dysfunctional disorder.

65. The method of claim 64, wherein the ancillary agent is a chemotherapeutic agent.

66. The method of claim 65, wherein the chemotherapeutic agent is an agent that targets rapidly dividing cells and/or disrupt the cell cycle or cell division.

67. The method of claim 65 or claim 66, wherein the chemotherapeutic agent is a cytotoxic agent.

68. The composition of claim 67, wherein the cytotoxic agent is taxane.

69. The composition of claim 68, wherein the taxane is paclitaxel.

70. The composition of claim 68, wherein the taxane is Abraxane.

71. A method of treating or delaying the progression of cancer in a subject, the method comprising, consisting or consisting essentially of administering concurrently to the subject a LSD inhibitor and a PD-1 binding antagonist in effective amounts to treat or delay the progression of the cancer.

72. The method of claim 71, wherein the subject has been diagnosed with cancer, wherein a T-cell in a tumor sample of the cancer from the subject expresses nuclear LSD at a higher level than the level of expression of TBET in the same T-cell, and/or at a higher level than in an activated T-cell.

73. The method of claim 71 or claim 72, further comprising administering concurrently to the subject, with the LSD inhibitor and the PD-1 binding antagonist, an ancillary agent (e.g., a chemotherapeutic agent) or ancillary therapy (e.g., ablation or cytotoxic therapy) for treating, or delaying the progression of the cancer.

74. A method of enhancing immune function (e.g., immune effector function) in an individual having cancer, the method comprising, consisting or consisting essentially of administering concurrently to the individual a LSD inhibitor and a PD-1 binding antagonist in effective amounts to enhance the immune function.

75. The method of claim 74, wherein the individual has been diagnosed with cancer, wherein a T-cell in a tumor sample of the cancer taken from the individual expresses nuclear LSD at a higher level than the level of expression of TBET in the same T-cell, and/or at a higher level than in an activated T-cell.

76. The method of claim 74 or claim 75, further comprising administering concurrently to the subject, with the LSD inhibitor and the PD-1 binding antagonist, an ancillary agent (e.g., a chemotherapeutic agent) or ancillary therapy (e.g., ablation or cytotoxic therapy) for enhancing the immune function.

77. A method of treating infection (e.g., with a bacteria or virus or other pathogen), the method comprising, consisting or consisting essentially of administering concurrently to the individual a LSD inhibitor and a PD-1 binding antagonist in effective amounts to treat the infection.

78. The method of claim 77, wherein the infection is with virus and/or bacteria.

79. The method of claim 77, wherein the infection is with a pathogen.

80. The method of any one of claims 77 to 79, wherein the infection is an acute infection.

81. The method of any one of claims 77 to 80, wherein the infection is a chronic infection.

82. The method of any one of claims 77 to 81, further comprising administering concurrently to the subject, with the LSD inhibitor and the PD-1 binding antagonist, an ancillary agent (e.g., a chemotherapeutic agent) or ancillary therapy (e.g., ablation or cytotoxic therapy) for treating the infection.

83. A method of enhancing immune function (e.g., immune effector function, T-cell function etc.) in an individual having an infection the method comprising, consisting or consisting essentially of administering concurrently to the individual a LSD inhibitor and a PD-1 binding antagonist in effective amounts to enhance the immune function.

84. The method of claim 83, wherein the individual has been diagnosed with the infection, wherein a T-cell in a sample taken from the individual expresses nuclear LSD at a higher level than the level of expression of TBET in the same T-cell, and/or at a higher level than in an activated T-cell.

85. The method of claim 83 or claim 84, further comprising administering concurrently to the subject, with the LSD inhibitor and the PD-1 binding antagonist, an ancillary agent (e.g., a chemotherapeutic agent) or ancillary therapy (e.g., ablation or cytotoxic therapy) for enhancing the immune function.

86. Use of a LSD inhibitor and a PD-1 binding antagonist for treating a T-cell dysfunctional disorder, or for enhancing immune function (e.g., immune effector function, T-cell function etc.) in an individual having cancer, for treating or delaying the progression of cancer, or for treating infection.

87. Use of a LSD inhibitor and a PD-1 binding antagonist in the manufacture of a medicament for treating a T-cell dysfunctional disorder, or for enhancing immune function (e.g., immune effector function, T-cell function etc.) in an individual having cancer, for treating or delaying the progression of cancer, or for treating infection.

88. The use of claim 86 or claim 87, wherein the LSD inhibitor and the PD-1 binding antagonist are formulated for concurrent administration.

89. Use of a LSD inhibitor, a PD-1 binding antagonist and an ancillary agent (e.g., a chemotherapeutic agent) for treating, or for aiding in the treatment of, a T-cell dysfunctional disorder, or for enhancing immune function (e.g., immune effector function, T-cell function etc.) in an individual having cancer, for treating or delaying the progression of cancer, or for treating infection.

90. Use of a LSD inhibitor, a PD-1 binding antagonist and an ancillary agent (e.g., a chemotherapeutic agent) in the manufacture of a medicament for treating, or for aiding in the treatment of, a T-cell dysfunctional disorder, or for enhancing immune function (e.g., immune effector function, T-cell function etc.) in an individual having cancer, for treating or delaying the progression of cancer, or for treating infection.

91. The use of claim 89 or claim 90, wherein the LSD inhibitor, PD-1 binding antagonist and ancillary agent (e.g., a chemotherapeutic agent) are formulated for concurrent administration.

92. The method of any one of claims 36 to 85, further comprising detecting an elevated level of nuclear LSD (i.e., a LSD localized in the nucleus) in a T cell (e.g., relative to the level of TBET in the same T-cell or the level of nuclear LSD in an activated T-cell) in a sample obtained from the subject, prior to the concurrent administration.

93. The method of any one of claims 36 to 85, further comprising detecting an elevated level of nuclear LSD (i.e., a LSD localized in the nucleus) in a T cell (e.g., relative to the level of TBET in the same T-cell or the level of nuclear LSD in an activated T-cell) and an elevated level of EOMES in the nucleus of the T cell (e.g., relative to the level of TBET in the same T-cell or the level of EOMES in the nucleus of an activated T-cell) in a sample obtained from the subject, prior to the concurrent administration.

94. The method of claim 93, comprising detecting an elevated level of a complex comprising a LSD and EOMES.

95. The method of claim 93, comprising detecting an elevated level of a complex comprising a LSD and EOMES in the nucleus of the T-cell.

96. A kit comprising a medicament comprising a LSD inhibitor and an optional pharmaceutically acceptable carrier, and a package insert comprising instructional material for concurrent administration of the medicament with another medicament comprising a PD-1 binding antagonist and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual.

97. A kit comprising a medicament comprising a PD-1 binding antagonist and an optional pharmaceutically acceptable carrier, and a package insert comprising instructional material for concurrent administration of the medicament with another medicament comprising a LSD inhibitor and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual.

98. A kit comprising a first medicament comprising a LSD inhibitor and an optional pharmaceutically acceptable carrier, and a second medicament comprising a PD-1 binding antagonist and an optional pharmaceutically acceptable carrier for treating a T-cell dysfunctional disorder, or for enhancing immune function (e.g., immune effector function, T-cell function etc.) in an individual having cancer, for treating or delaying the progression of cancer, or for treating infection in an individual.

99. The kit of claim 98, further comprising a package insert comprising instructional material for administering concurrently the first medicament and the second medicament for treating a T-cell dysfunctional disorder, or for enhancing immune function (e.g., immune effector function, T-cell function etc.) in an individual having cancer, for treating or delaying the progression of cancer, or for treating infection in an individual.

100. The method of any one of claims 36 to 85, wherein CD8⁺ T cells in the individual have enhanced priming, activation, proliferation and/or cytolytic activity as compared to before the administration of the combination.

101. The method of any one of claims 36 to 85 and 100, wherein the number of CD8⁺ T cells is elevated as compared to before administration of the combination.

102. The method of claim 101, wherein the CD8⁺ T cell is an antigen-specific CD8+ T cell.

103. The method of any one of claims 36 to 85 and 100 to 102, wherein Treg function is suppressed as compared to before administration of the combination of the LSD inhibitor and PD-1 binding antagonist.

104. The method of any one of claims 36 to 85 and 100 to 103, wherein T cell exhaustion is decreased as compared to before administration of the combination of the LSD inhibitor and PD-1 binding antagonist.

105. The method of any one of claims 36 to 85 and 100 to 104, wherein number of Treg cells is decreased as compared to before administration of the combination of the LSD inhibitor and PD-1 binding antagonist.

106. The method of any one of claims 36 to 85 and 100 to 105, wherein plasma IFN-γ is increased as compared to before administration of the combination of the LSD inhibitor and PD-1 binding antagonist.

107. The method of any one of claims 36 to 85 and 100 to 106, wherein plasma TNF-α is increased as compared to before administration of the combination of the LSD inhibitor and PD-1 binding antagonist.

108. The method of any one of claims 36 to 85 and 100 to 107, wherein plasma IL-2 is increased as compared to before administration of the combination of the LSD inhibitor and PD-1 binding antagonist.

109. The method of any one of claims 36 to 85 and 100 to 108, wherein the number of memory T effector cells is increased as compared to before administration of the combination of the LSD inhibitor and PD-1 binding antagonist.

110. The method of any one of claims 36 to 85 and 100 to 109, wherein memory T effector cell activation and/or proliferation is increased as compared to before administration of the combination of the LSD inhibitor and PD-1 binding antagonist.

111. The method of any one of claims 36 to 85 and 100 to 110, wherein memory T effector cells are detected in peripheral blood.

112. The method of claim 111, wherein detection of memory T effector cells is by detection of CXCR3.

113. A method of diagnosing the presence of a T-cell dysfunctional disorder in a subject, the method comprising, consisting or consisting essentially of:
(i) obtaining a sample from the subject, wherein the sample comprises a T-cell (e.g., CD8⁺ T-cell or CD4⁺ T-cell);
(ii) contacting the sample with a first binding agent that binds to LSD in the sample and a second binding agent that binds to EOMES in the sample; and
(iii) detecting localization of the first and second binding agents in the nucleus of the T-cell;
wherein localization of the first and second binding agents in the nucleus of the T-cell is indicative of the presence of the T-cell dysfunctional disorder in the subject.

114. A method of diagnosing the presence of a T-cell dysfunctional disorder in a subject, the method comprising, consisting or consisting essentially of:
(i) obtaining a sample from the subject, wherein the sample comprises a T-cell (e.g., CD8⁺ T-cell or CD4⁺ T-cell);
(ii) contacting the sample with a first binding agent that binds to LSD in the sample and a second binding agent that binds to EOMES in the sample; and
(iii) detecting the first and second binding agents when bound to a LSD-EOMES complex in the sample;
wherein an elevated level of LSD-EOMES complex detected in the sample relative to a level of LSD-EOMES complex detected in a control sample (e.g., one comprising an activated T-cell) is indicative of the presence of the T-cell dysfunctional disorder in the subject.

115. A method of monitoring the treatment of a subject with a T-cell dysfunctional disorder, the method comprising, consisting or consisting essentially of:
(i) obtaining a sample from the subject following treatment of the subject with a therapy for the T-cell dysfunctional disorder, wherein the sample comprises a T-cell (e.g., CD8⁺ T-cell or CD4⁺ T-cell);
(ii) contacting the sample with a first binding agent that binds to LSD in the sample and a second binding agent that binds to EOMES in the sample; and
(iii) detecting the first and second binding agents when bound to a LSD-EOMES complex in the sample;
wherein a lower level of LSD-EOMES complex detected in the sample relative to a level of LSD-EOMES complex detected in a control sample taken from the subject prior to the treatment is indicative of an increased clinical benefit (e.g., enhanced immune effector function such as enhanced T-cell function) to the subject, and
wherein a higher level of LSD-EOMES complex detected in the sample relative to a level of LSD-EOMES complex detected in a control sample taken from the subject prior to the treatment is indicative of no or negligible clinical benefit (e.g., enhanced immune effector function such as enhanced T-cell function) to the subject.

116. A kit for diagnosing the presence of a T-cell dysfunctional disorder in a subject, the kit comprising, consisting or consisting essentially of: (i) a first binding agent that binds to LSD, (ii) a second binding agent that binds to EOMES; and (iii) a third agent comprising a label, which is detectable when each of the first and second binding agents is bound to a LSD-EOMES complex.

117. The kit of claim 116, wherein the third agent is a binding agent that binds to the first and second binding agent.

118. A complex comprising a LSD and EOMES, a first binding agent that is bound to LSD of the complex, a second binding agent bound to EOMES of the complex; and (iii) a third agent comprising a label, which is detectable when each of the first and second binding agents is bound to the LSD-EOMES complex.

119. The complex of claim 118, wherein the LSD-EOMES complex is located in a T-cell.

120. The complex of claim 118 or claim 119, wherein the third agent is a binding agent that binds to the first and second binding agent.

121. A T-cell that comprises a complex comprising a LSD and EOMES, a first binding agent that is bound to LSD of the complex, a second binding agent bound to EOMES of the complex; and (iii) a third agent comprising a label, which is detectable when each of the first and second binding agents is bound to the LSD-EOMES complex.

122. The T-cell of claim 121, wherein the third agent is a binding agent that binds to the first and second binding agent.

123. A method, kit, complex or T-cell according to any one of claims 116 to 122, wherein respective binding agents are antibodies.
